(19) United States
(12) Patent Application Publication
(10) Pub. No.: US 2026/0114817 A1
(43) Pub. Date: Apr. 30, 2026

(54) SYSTEMS AND METHODS FOR DETECTING A PHYSIOLOGICAL FEATURE OF A SUBJECT

(71) Applicant: Eight Sleep Inc., New York, NY (US)

(72) Inventors: Matteo FRANCESCHETTI, Miami Shores, FL (US); Alexandra ZATARAIN, Miami Shoers, FL (US); Massimo ANDREASI BASSI, Union City, NY (US); Brian George SCHIFFER, San Francisco, CA (US); David Da HE, San Carlos, CA (US); Jaewon Samuel KANG, Oakland, CA (US); David HEINZ, San Francisco, CA (US); Kevin Jacob KOTZEN, San Diego, CA (US); John Norman MAIDENS, Edmonton (CA); Niranjan SRIDHAR, San Mateo, CA (US)

(21) Appl. No.: 19/381,720

(22) Filed: Nov. 6, 2025

Related U.S. Application Data
(63) Continuation of application No. PCT/US25/16900, filed on Feb. 21, 2025.
(60) Provisional application No. 63/557,256, filed on Feb. 23, 2024.
(60) Provisional application No. 63/753,636, filed on Feb. 4, 2025.

(51) Int. Cl. A61B 5/00; A61B 5/0205; A61B 5/024; A61B 5/08

(52) U.S. Cl. CPC A61B 5/7275; A61B 5/0205; A61B 5/4806; A61B 5/6891; A61B 5/742; A61B 5/02405; A61B 5/0816

(57) ABSTRACT

An aspect of the present disclosure provides a system comprising a sensing device comprising a housing and at least one sensor in sensing communication with an outer environment of the housing. The at least one sensor can be configured to detect a user radiation signal that is reflected or emitted by at least a portion of a body of the subject. The system can further comprise a reference device comprising at least one radiation source configured to output a reference radiation signal. The user radiation signal and the reference radiation signal can be analyzed to determine a physiological feature of the subject.

### CROSS-REFERENCE TO RELATED APPLICATIONS

This application is a bypass continuation from International Patent Application No. PCT/US25/16900, filed Feb. 21, 2025, which claims the benefit to U.S. Provisional Patent Application No. 63/557,256, filed Feb. 23, 2024, and U.S. Provisional Patent Application No. 63/753,636, filed Feb. 4, 2025, each of which is entirely incorporated herein by reference.

### BACKGROUND

One or more biological signals of a subject can be detected while the subject is using an article of furniture (e.g., sleeping on a bed device). In some cases, the detected biological signal(s) can be used to determine a condition of the subject.

### SUMMARY

In one aspect, the present disclosure provides a system for monitoring a physiological feature of a subject, comprising: a sensing device comprising a housing and at least one sensor in optical communication with an outer environment of the housing, wherein the at least one sensor is configured to detect a user electromagnetic radiation signal that is reflected or emitted by at least a portion of a body of the subject; a reference device comprising at least one optical source configured to output a reference electromagnetic radiation signal; and a processor operatively coupled to the sensing device and the reference device, configured to: (a) direct the reference device to output the reference electromagnetic radiation signal; (b) direct the sensing device to detect the reference electromagnetic radiation signal and the user electromagnetic radiation signal; and (c) determine the physiological feature of the subject based on both of the reference electromagnetic radiation signal and the user electromagnetic radiation signal.

In one aspect, the present disclosure provides a method for monitoring a physiological feature of a subject, comprising: (a) outputting, using a reference device comprising an optical source, to output a reference electromagnetic radiation signal; (b) detecting, using a sensing device comprising a housing and at least one sensor in optical communication with an outer environment of the housing, (i) the reference electromagnetic radiation signal and (ii) a user electromagnetic radiation signal that is reflected or emitted by at least a portion of a body of the subject; and (c) determining the physiological feature of the subject based on both of the reference electromagnetic radiation signal and the user electromagnetic radiation signal.

In one aspect, the present disclosure provides a system for monitoring a physiological feature of a subject, comprising: an article of furniture comprising a contact sensor configured to detect a contact sensor signal associated with the subject when the subject is disposed over a top surface of the article of furniture; a sensing device comprising an optical sensor in optical communication with the top surface of the article of furniture, wherein the optical sensor is configured to detect a user electromagnetic radiation signal that is reflected or emitted by at least a portion of a body of the subject; and a processor operatively coupled to the contact sensor and the optical sensor, configured to: (a) direct the contact sensor to detect the contact sensor signal; (b) direct the optical sensor to detect the user electromagnetic radiation signal; (c) determine the physiological feature of the subject based on both of the contact sensor signal and the user electromagnetic radiation signal.

In one aspect, the present disclosure provides a method for monitoring a physiological feature of a subject, comprising: (a) detecting, using a contact sensor of an article of furniture, a contact sensor signal associated with the subject when the subject is disposed over a top surface of the article of furniture; (b) detecting, using a sensing device comprising an optical sensor in optical communication with the top surface of the article of furniture, a user electromagnetic radiation signal that is reflected or emitted by at least a portion of a body of the subject; and (c) determining the physiological feature of the subject based on both of the contact sensor signal and the user electromagnetic radiation signal.

In one aspect, the present disclosure provides a system for enhancing sleep of a user, the system comprising: a sleep enhancement device comprising a regulator and a bed device having an at least partially internal heating or cooling element, wherein the regulator is configured to adjust at least one sleep-related parameter for the bed device to enhance sleep of the user; and a projector configured to project an optical pattern to a wall or a ceiling adjacent the bed device, wherein the optical pattern provides at least one of (a) status information for the bed device, local environment, or both or (b) at least one control option to direct operation of the sleep enhancement device.

In one aspect, the present disclosure provides a computer-implemented method for enhancing sleep of a user, the method comprising: adjusting at least one sleep-related parameter for a bed device of a sleep enhancement device, thereby enhancing sleep of a user of the bed device, wherein the bed device comprises an at least partially internal heating or cooling element; and projecting, via a projector, an optical pattern to a wall or a ceiling adjacent the bed device, wherein the optical pattern provides at least one of (a) status information for the bed device, local environment, or both or (b) at least one control option to direct operation of the sleep enhancement device.

In one aspect, the present disclosure provides a computer-implemented method for detecting a condition of a user of an article of furniture, the method comprising: determining, by a first computer algorithm, one or more sensor data signatures indicative of a physiological condition of the user via based on an input sensor data associated with a biological signal of the user, wherein the input sensor data is collected by a biological sensor while the user is sleeping on the article of furniture; and confirming, by a second and different computer algorithm, the determined one or more sensor data signatures based on (i) at least a portion of the input sensor data and (ii) the determined one or more sensor data signatures.

In one aspect, the present disclosure provides a computer-implemented method for detecting an anomaly in a physiological condition of a user of an article of furniture, the method comprising: predicting a future sensor data or analysis thereof (future data) based on a historical sensor data set associated with the user of an article of furniture, wherein the future data is indicative of a physiological condition of the user; and determining a difference between the predicted future data and a new data or analysis data thereof (new data), wherein the difference is indicative of the anomaly in the physiological condition of the user.

In one aspect, the present disclosure provides a computer-implemented method for detecting a condition of a user of an article of furniture, the method comprising: detecting a plurality of sensor data signatures from a sensor data set associated with a user of an article of furniture; identifying one or more acceptable quality sensor data signatures from the plurality of sensor data signatures via a signal quality filtering algorithm; and determining presence of the physiological condition of the user based on the one or more acceptable quality sensor data signatures identified.

Another aspect of the present disclosure provides a system comprising: a computer processor and a computer memory coupled thereto, wherein the computer memory comprises a machine executable code that, upon execution by the one or more computer processors, implements the methods described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example system for monitoring a physiological feature of a subject.

FIG. 2 schematically illustrates an additional example system for monitoring a physiological feature of a subject.

FIG. 3 illustrates an example of a bed device containing a projector displaying data on a ceiling.

FIG. 4 illustrates an example of a side view of a sleep enhancement system.

FIG. 5 illustrates an example of a front view of the sleep enhancement system.

FIG. 6 illustrates an example of the sleep enhancement system containing projection geometries.

FIG. 7 illustrates an example of a side view of the sleep enhancement system containing field of view.

FIG. 8 illustrates an example of a front view of the sleep enhancement system containing field of view.

FIG. 9 illustrates an example of a schematic view of a housing containing the projector and sensors.

FIGS. 10A-10B show exemplary outputs of the longitudinal health monitoring described herein. FIG. 10A shows data from a user depicting sleep quality and sleep duration of a previous night's sleep session. The representation shows minimal anomalies for breathing detection, heartbeat detection, and wellbeing detection. FIG. 10B shows data from a user depicting sleep quality and sleep duration of a previous night's sleep session. The representation shows multiple anomalies for breathing detection, heartbeat detection, and wellbeing detection.

FIGS. 11A-11B show visualizations of breathing disturbance detection. FIG. 11A shows a daily view of average breathing anomalies per hour over one sleep session. The plot depicts the number of anomalies per hour and the user can see a time when the number of anomalies may fall out of the optimal range. FIG. 11B shows a monthly view of average breathing anomalies per hour. The plot depicts the average number of breathing anomalies per hour over 28 days.

FIG. 12 shows an exemplary visualization of heartbeat anomaly data.

FIGS. 13A-13B show exemplary Poincaré plots that depict distribution of measured interbeat intervals (IBIs). FIG. 13A shows plots depicting Afib patterns with the left plot depicting outliers prior to removal of respiratory sinus arrhythmia (RSA) and the right plot depicting 17.37% outliers following removal of respiratory sinus arrhythmia (RSA). FIG. 13B shows plots depicting RSA (benign) patterns. After removal of respiratory sinus arrhythmia (RSA), almost no irregular beats remained (2.02% outliers).

FIG. 14A-14B show exemplary Poincaré plots that depict distribution of measured interbeat intervals (IBIs). FIG. 14A shows phase-dependent irregularities. Following RSA removal, the irregularity clusters moved to the center cone area. FIG. 14B shows RSA and phase-dependent irregularities

FIG. 15 shows an exemplary visualization of wellbeing monitoring. The representation shows an output screen with data on heart rate, respiratory rate, and HRV for a day.

FIG. 16 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most,” “up to,” “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

When ranges are present, the ranges include the range endpoints. Additionally, every sub range and value within the range is present as if explicitly written out. The terms “about” and “approximately,” as used herein, when preceding a numerical value indicates the value plus or minus a range of 10%. For example, about 10 may be reasonably understood to convey 9, 10, or 11, or a range of numerical values spanning from 9 to 11. Whenever “about” or “approximately” precedes the first numerical value in a series of two or more numerical values, the term “about” or “approximately” applies to each of the numerical values in that series of numerical values.

Further, these components can execute from various computer readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

As another example, a component can be an apparatus with specific functionality provided by mechanical parts operated by electric or electronic circuitry; the electric or electronic circuitry can be operated by a software application or a firmware application executed by one or more processors; the one or more processors can be internal or external to the apparatus and can execute at least a part of the software or firmware application. As yet another example, a component can be an apparatus that provides specific functionality through electronic components without mechanical parts; the electronic components can include one or more processors therein to execute software and/or firmware that confer(s), at least in part, the functionality of the electronic components. In some embodiments, a component can emulate an electronic component via a virtual machine, e.g., within a cloud computing system.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

The terms “furniture,” “article of furniture,” or “piece of furniture,” as used interchangeably herein, generally refer to a bed, a pillow, crib, bassinet, chair, seat, loveseat, sofa, couch, head rest, stool, ottoman, bench, or any panel intended to be covered with a fabric. The article of furniture can be intended for use in a home, an office, a medical facility (e.g., a hospital), or on a vehicle of transportation such as a car, truck, boat, bus, train or the like. The article of furniture can be intended for use for at least one person (and/or at least one animal, such as a pet). The article of furniture can be intended for use for at least about 1, 2, 3, 4, 5, 6, 7, 8, 9, 10, or more persons. The article of furniture can be intended for use for at most about 10, 9, 8, 7, 6, 5, 4, 3, 2, or 1 person. In an example, the article of furniture may be a bed, and the bed may comprise a plurality of sizes comprising single, single extra-long, double, queen, king, super king, etc. In another example, the article of furniture may be an infant warmer (i.e., a babytherm) to provide heat at one or more temperatures to an infant.

The terms “bed” or “bed device,” as used interchangeably herein, may be an article of furniture used for sleep or rest. The bed may comprise a mattress, a mattress pad, a pillow, and/or a covering thereof (e.g., a blanket). One or more users may sleep or rest on and/or adjacent to a surface of the bed device. The surface may be a top surface of the bed device. The top surface of the bed device may be flat or textured. The bed device may be a mattress. The bed device may be a mattress pad that covers at least a portion of a surface of a mattress or at least a surface of the mattress. The bed device may be a pillow. Alternatively or in addition to, the user(s) may sleep under a surface of the bed device. The surface may be one or more surfaces of a covering, such as, for example, a blanket. The blanket may be disposed on top of at least a part of the user(s). The bed device may be the blanket.

A temperature of the article of furniture (e.g., the bed device, such as the mattress, the mattress pad, pillow, or the blanket) may be controlled (e.g., increasing, decreasing, or maintaining the temperature of the bed). A temperature of at least a portion of the article of furniture may be controlled. The temperature of the article of furniture may be adjustable or maintained prior to, during, or subsequent to a use (e.g., sleeping or resting for a period of time) by the user(s). In an example, the bed may be pre-warmed (e.g., automatically or per user preference) prior to the use by the user(s). In some cases, temperatures or two or more portions of the article of furniture (e.g., the bed) may be controlled separately or in sync.

The article of furniture (e.g., the bed) may use one or more sensors and/or one or more computer systems to detect sensing data (e.g., one or more biological signals) associated with the user. For example, the sensing data can be utilized to estimate or determine a condition or state of the user prior to, during, or subsequent to using the article of furniture (e.g., determine sleep phase, sleep pattern, disease, disorder, snoring, etc. of the user). The sensor(s) may or may not be a part of the article of furniture. The sensor(s) may be part of the article of furniture. The sensor(s) may be a part of a space (e.g., room) surrounding the article of furniture. The sensor(s) may be worn by the user(s). Non-limiting examples of a sensor can include a capacitance sensor, a temperature sensor, a pressure sensor, a piezoelectric sensor, sound sensor (e.g., a microphone), accelerometer, liquid pressure sensor, etc. The sensing data can be utilized (e.g., analyzed), at least in part, to determine how to regulate temperature of the article of furniture prior to, during, and subsequent to the user's use of the article of furniture. In some cases, the sensor(s) may be used to detect a property (e.g., temperature, movement, etc.) of the article of furniture or such property of an environment surrounding the article of furniture. The sensor(s) of the article of furniture (e.g., coupled to, integrated into, embedded into, etc.) as provided herein can detect the sensing data when in contact (e.g., direct or indirect contact) with the subject. Accordingly, in some cases, the sensor(s) of the article of furniture can be referred to as contact sensor(s). In some cases, a sensor can be referred to as a sensing device, and the terms “sensor” and “sensing device” may be used interchangeably.

The term “sleep phase,” as used herein, can refer to a light sleep, deep sleep, or rapid eye movement (“REM”) sleep. There can be two major stages of sleep: a non-REM sleep and a REM sleep. A person can experience a non-REM sleep first, followed by a shorter period of REM sleep. In some cases, the person can experience a continued cycle of the non-REM sleep and the REM sleep. There may be three stages of non-REM sleep. Each stage can last from 5 to 15 minutes. The person can go through all three stages before reaching REM sleep. In stage one, the person's eyes may be closed, but the person may be easily woken up. This stage may last for 5 to 10 minutes. This stage may be considered as a light sleep. In stage two, the person may be in light sleep. The person's heart rate may slow and the person's body temperature may drop. The person's body may be getting ready for deep sleep. This stage may also be considered as a light sleep. Stage three may be a deep sleep stage. The person may be harder to rouse during this stage, and if the person was woken up, the person would feel disoriented for a few minutes. During the deep stage of the non-REM sleep, the body may repair and regrow tissues, build bone and muscle, and strengthen the immune system. The REM sleep can happen 90 minutes after a person falls asleep. In some cases, the person may have dreams during the REM sleep. An initial period of the REM sleep may typically last 10 minutes. Any latter period of the REM sleep may get longer, and the final period of the REM sleep may last up to about an hour. The person's heart rate and respiration may quicken during the REM sleep (e.g., during the final period of the REM sleep). The person may have intense dreams during the REM sleep, since the brain is more active. The REM sleep may affect learning of certain mental skills.

A “sleep pattern”, as used herein, can indicate a recurrence or change in (i) one or more biological signals and/or (i) one or more sleep phases of the user of the bed. The sleep pattern may be described over a period of time (e.g., 0.5 hours, 1 hour, 2 hours, 3 hours, 4 hours, 5 hours, 6 hours, etc.), along with a count of the biological signal(s) or the sleep phase(s). The sleep pattern may comprise a preferred setting of the biological signal(s) or sleep phase(s) of the user. The preferred setting of the biological signal(s) may comprise a type of the biological signal(s), along with a preferred value or range of values of the biological signal(s) (e.g., a preferred body temperature or range of body temperature of the user). The preferred setting of the sleep phase(s) may comprise a type of the sleep phase(s), along with a preferred value or range of values of the sleep phase(s).

In some embodiments, the sensor(s) of the article of furniture can be disposed within a portion of the article of furniture that corresponds to a target bodily portion of the user, such as head, arms, legs, torso, upper body, lower body, etc.

A disorder of a user can be a sleep disorder. Non-limiting examples of the sleep disorder may include dyssomnias, such as insomnia, primary hypersomnia (e.g., narcolepsy, idiopathic hypersomnia, recurrent hypersomnia, posttraumatic hypersomnia, menstrual-related hypersomnia), sleep disordered breathing (e.g., sleep apnea, snoring, upper airway resistance syndrome), circadian rhythm sleep disorders (e.g., delayed sleep phase disorder, advanced sleep phase disorder, non-24-hour sleep-wake disorder), parasomnias (e.g., bedwetting, bruxism, catathrenia, exploding head syndrome, sleep terror, rapid eye movement (REM) sleep behavior disorder, sleep talking, jet lag, restless legs syndrome, sleep deficiency (e.g., over the course of days, weeks, months, etc.), etc.

In some embodiments, the article of furniture can be a bed (e.g., a mattress or a mattress cover), and the temperature of the bed can be controlled (e.g., based at least in part on the sensing data) to assist the user(s) to fall asleep, assist the user to wake up from sleeping, promote enhanced sleep quality, treat or ameliorate the disorder of the user while sleeping, etc.

The terms “biological signal” and “bio signal” can be used interchangeably. Examples of the biological signal can include a heart signal (e.g., heart rate variability (HRV), heart rate, time elapsed between two successive R-wave peaks (R-R interval), or sound), a respiration (breathing) signal (e.g., respiration rate or sound), a motion, a temperature, a movement, perspiration, sound, neural activity, blood oxygenation (e.g., as measured from directly or indirectly contacting the skin of the user, etc. The article of furniture (e.g., the bed) may be capable of detecting one or more biological signals of the user(s). For example, the sensing data obtained by the sensor can comprise ballistocardiography (BCG) data or alike, or electrocardiogram (ECG) data or alike. The article of furniture may be capable of adjusting a property of the article of furniture (e.g., temperature or movement of the article of furniture, such as vibration, geometric configuration, etc.) to control (e.g., increase, decrease, or maintain) the biological signal(s) of the user(s) of the article of furniture.

The term “module” refers broadly to software, hardware, or firmware components (or any combination thereof). Modules are typically functional components that can generate useful data or another output using specified input(s). A module may or may not be self-contained. An application program (also called an “application”) may include one or more modules, or a module may include one or more application programs.

The term “on top of” can mean that the two objects, where the first object is “on top of” the second object, can be rotated so that the first object is above the second object relative to the ground. The two objects can be in direct or indirect contact, or may not be in contact at all.

The term “real time” or “real-time,” as used interchangeably herein, generally refers to an event (e.g., an operation, a process, a method, a technique, a computation, a calculation, an analysis, an optimization, etc.) that is performed using recently obtained (e.g., collected or received) data.

Examples of the event may include, but are not limited to, analysis of sensing data (e.g., one or more biological signals), adjusting a condition of an article of furniture, etc. In some cases, a real-time event may be performed almost immediately or within a short enough time span, such as within at least 0.0001 milliseconds, 0.0005 milliseconds, 0.001 milliseconds, 0.005 milliseconds, 0.01 milliseconds, 0.05 milliseconds, 0.1 milliseconds, 0.5 milliseconds, 1 millisecond, 5 milliseconds, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 milliseconds, 1 millisecond, 0.5 milliseconds, 0.1 milliseconds, 0.05 milliseconds, 0.01 milliseconds, 0.005 milliseconds, 0.001 milliseconds, 0.0005 milliseconds, 0.0001 milliseconds, or less. Real time can refer to a response time of less than 1 second, tenth of a second, hundredth of a second, a millisecond, or less, such as by a computer processor. Real-time can also refer to a simultaneous or substantially simultaneous occurrence of a first event with respect to occurrence of a second event. One or more operations in the present disclosure can be performed in real-time, near real-time, or substantially real-time.

### Computer Vision Systems and Methods Thereof for Monitoring a Subject

Various aspects of the present disclosure provide systems comprising sensors (e.g., optical sensors) for monitoring a physiological feature (e.g., a condition or state) of a subject, and methods thereof. In some embodiments, the monitored physiological feature can be utilized to determine a health condition (e.g., disease) of the subject. In some embodiments, the monitored physiological feature can be utilized to control (e.g., automatically control) operation of an article of furniture that the subject is using (e.g., a bed device that the subject is sleeping on) and/or other devices associated with the user (e.g., a light, an alarm, a coffee machine, a lock, a user device such as a mobile phone, a personal computer, a smart watch, etc.).

In some embodiments, the system can comprise a sensing device comprising a housing and at least one sensor in sensing communication with an outer environment of the housing. The at least one sensor can be configured to detect a user signal (e.g., user radiation signal) that is reflected or emitted by at least a portion of a body of the subject. The at least one sensor can be configured to detect a user signal that is reflected or emitted by at least or at most about 1%, 5%, 10%, 15%, 20%, 30%, 40%, 50%, 60%, 70%, 80%, 90%, 95%, 99%, or substantially 100% of the at least the portion of the body of the subject. The at least the portion of the body of the subject can comprise a head, arm(s), hand(s), torso, legs, foot/feet, etc. At least a portion of the at least one sensor can be coupled to or disposed on an outer surface of the housing, to achieve sensing communication with the outer environment of the housing. The at least one sensor can be a single sensor. Alternatively, the at least one sensor can comprise a plurality of sensors (e.g., sensors of the same type, or sensors of different types), e.g., at least or at most about 2, 3, 4, 5, 6, 7, 8, 9, or 10 sensors.

In some embodiments, the system can comprise a reference device comprising at least one signal source (e.g., at least one radiation source) configured to output a reference signal (e.g., reference radiation signal). The reference signal can be detected by the at least one sensor of the sensing device as a point of reference prior to, simultaneously with, or subsequent to the detection of the user signal. In some cases, the reference signal can be utilized to optimize or calibrate the at least one sensor prior to its use. In some cases, the reference signal can be utilized as a point of reference (e.g., a known or predetermined signal) when analyzing the detected user signal (e.g., user radiation signal). Without wishing to be bound by theory, use of the reference signal can provide more accurate measurement and/or analysis of the user signal than without use of the reference signal.

In some embodiments, the at least one signal source can be configured to output a single reference signal (e.g., a single electromagnetic wavelength or a single acoustic or ultrasound wavelength). In some embodiments, the at least one signal source can be configured to output a plurality of reference signals (e.g., a plurality of difference electromagnetic wavelengths or a plurality of different acoustic or ultrasound wavelengths). For example, the plurality of reference signals can include an expected low threshold value and an expected high threshold value of the user signal, thereby providing a reference spectrum to compare the detected user signal. The plurality of reference signals can be provided by a single signal source (e.g., a single optical source or a single speaker) or two or more different signal sources (e.g., different optical sources or different speakers). In some cases, values (e.g., wavelength or frequency values) of two or more reference signals can be at least or at most about 1%, 5%, 10%, 15%, 20%, 30%, 40%, 50%, 60%, 70%, 80%, 90%, 95%, 100%, 120%, 150%, 200%, 300%, 400%, 500%, 600%, 700%, 800%, 900%, or 1,000%.

In some embodiments, at least one physiological feature (e.g., a single physiological feature, a plurality of physiological features) of the subject can be determined based on both of the reference signal and the user signal. In some cases, data comprising the reference signal and the user signal can be processed (e.g., analyzed) to determine the at least one physiological feature. In some cases, the data processing can be performed partially or entirely by one or more classifiers (e.g., one or more machine learning algorithms).

Non-limiting examples of operations of data processing can include filtering, linear filtering, nonlinear filtering, folding, grouping, energy computation, lowpass filtering, bandpass filtering, highpass filtering, median filtering, rank filtering, quartile filtering, percentile filtering, mode filtering, finite impulse response (FIR) filtering, infinite impulse response (IIR) filtering, moving average (MA) filtering, autoregressive (AR) filtering, autoregressive moving averaging (ARMA) filtering, selective filtering, adaptive filtering, interpolation, decimation, subsampling, upsampling, resampling, time correction, time base correction, phase correction, magnitude correction, phase cleaning, magnitude cleaning, matched filtering, enhancement, restoration, denoising, smoothing, signal conditioning, enhancement, restoration, spectral analysis, linear transform, nonlinear transform, inverse transform, frequency transform, inverse frequency transform, Fourier transform (FT), discrete time FT (DTFT), discrete FT (DFT), fast FT (FFT), wavelet transform, Laplace transform, Hilbert transform, Hadamard transform, trigonometric transform, sine transform, cosine transform, DCT, power-of-2 transform, sparse transform, graph-based transform, graph signal processing, fast transform, a transform combined with zero padding, cyclic padding, padding, zero padding, feature extraction, decomposition, projection, orthogonal projection, non-orthogonal projection, over-complete projection, eigen-decomposition, singular value decomposition (SVD), principle component analysis (PCA), independent component analysis (ICA), grouping, sorting, thresholding, soft thresholding, hard thresholding, clipping, soft clipping, first derivative, second order derivative, high order derivative, convolution, multiplication, division, addition, subtraction, integration, maximization, minimization, least mean square error, recursive least square, constrained least square, batch least square, least absolute error, least mean square deviation, least absolute deviation, local maximization, local minimization, optimization of a cost function, neural network, recognition, labeling, training, clustering, machine learning, supervised learning, unsupervised learning, semi-supervised learning, comparison with another TSCI, similarity score computation, quantization, vector quantization, matching pursuit, compression, encryption, coding, storing, transmitting, normalization, temporal normalization, frequency domain normalization, classification, clustering, labeling, tagging, learning, detection, estimation, learning network, mapping, remapping, expansion, storing, retrieving, transmitting, receiving, representing, merging, combining, splitting, tracking, monitoring, matched filtering, Kalman filtering, particle filter, intrapolation, extrapolation, histogram estimation, importance sampling, Monte Carlo sampling, compressive sensing, representing, merging, combining, splitting, scrambling, error protection, forward error correction, doing nothing, time varying processing, conditioning averaging, weighted averaging, arithmetic mean, geometric mean, harmonic mean, averaging over selected frequency, averaging over antenna links, logical operation, permutation, combination, sorting, AND, OR, XOR, union, intersection, vector addition, vector subtraction, vector multiplication, and vector division. In some cases, one or more of the operations of data processing as provided herein can be performed via one or more machine learning algorithms (e.g., one or more trained classifiers). In some cases, one or more of the operations of data processing as provided herein can be performed by a computer processor without utilizing a machine learning algorithms.

In some embodiments, the physiological feature of the subject can be determined based on a difference or similarity in values (e.g., wavelengths, frequencies) between the reference signal (e.g., the reference radiation signal such as the reference electromagnetic radiation signal) and the user signal (e.g., the user radiation signal such as the user electromagnetic radiation signal). In some embodiments, a difference between values (e.g., about 1%, 2%, 5%, 10%, 20%, 50%, 90%, etc.) of the reference signal and the user signal can be calculated, and the calculated difference data can be utilized (e.g., as opposed to the absolute value of the user signal) to determine physiological feature of the subject. Alternatively or in addition to, other processing means as provided herein (e.g., addition, multiplications, averaging, thresholding, etc.) to process the reference signal and the user signal, to generate a new signal data which can be utilized to determine physiological feature of the subject.

In some embodiments, an image and/or video can be generated based on the user signal and the reference signal. The image and/or video can be multi-dimensional, e.g., two-dimensional or three-dimensional. In some embodiments, an image and/or video can be generated based on the user electromagnetic radiation signal and the reference electromagnetic radiation signal. The image and/or video can be a representative illustration of the processing of (e.g., analysis of) the user radiation signal and the reference radiation signal. For example, the user radiation signal, the reference radiation signal, and/or a processed value thereof (e.g., difference thereof) may not comprise a visible electromagnetic radiation wavelength, and such data can be further processed to be transformed or translated into the visible electromagnetic spectrum to generate the image and/or video that is visible to the naked human eye. Alternatively, such transformation or translation may not be needed to generate the image and/or video. In some embodiments, the image and/or video can be stored in a database and/or provided to a user or a third party associated with the user (e.g., family members, physicians, etc.) via a graphical user interface (GUI).

In some embodiments, the image and/or video can be analyzed to monitor or determine the physiological condition of the user. In some embodiments, the image and/or video can be a representation of a part or whole-body image of the subject (or user). For example, the image and/or video can be a thermal image and/or video, in which infrared radiation (or heat) is converted into visible image/or video that depicts the spatial distribution of temperature differences in a scene (e.g., at least a portion of the body of the subject) that is detected or viewed by the at least one sensor, e.g., with or without use of the reference signal.

In some embodiments, the user signal and/or the reference signal can comprise an electromagnetic radiation wavelength or wavelength range thereof. The electromagnetic radiation wavelength can comprise one or more wavelengths including, but not limited to, x-rays (e.g., about 0.1 nanometers (nm) to about 10.0 nm; or about 10\hat{ }18 hertz (Hz) to about 10\hat{ }16 Hz), ultraviolet (UV) rays (e.g., about 10.0 nm to about 380 nm; or about 8×10\hat{ }16 Hz to about 10\hat{ }15 Hz), visible light (e.g., about 380 nm to about 750 nm; or about 8×10\hat{ }14 Hz to about 4×10\hat{ }14 Hz), infrared light (e.g., about 750 nm to about 0.1 centimeters (cm); or about 4×10\hat{ }14 Hz to about 5×10\hat{ }11 Hz), and microwaves (e.g., about 0.1 cm to about 100 cm; or about 10\hat{ }8 Hz to about 5×10\hat{ }11 Hz), and other radio waves (e.g., about 1 meters to about 100 kilometers; or about 300 megahertz to about 3 kilohertz). Within the wavelength range of the UV rays, wavelengths of about 300 nm to about 380 nm may be referred to as “near” ultraviolet, wavelengths of about 200 nm to about 300 nm as “far” ultraviolet, and 10 about to about 200 nm as “extreme” ultraviolet. In some cases, within the wavelength range of the visible light, wavelengths of about 380 nm to about 490 nm may be referred to as “blue” light. The infrared light may comprise one or more ranges selected from the group consisting of: (i) near-infrared (NIR; from about 750 nm to about 1 micrometer (μm)), (ii) short-wavelength infrared (SWIR; from about 1 micrometer (μm) to about 3 μm), (iii) mid-wavelength infrared (MWIR; from about 3 μm to about 8 μm), (iv) long-wavelength infrared (LWIR; from about 8 μm to about 15 μm), and (v) far infrared (FIR; from about 15 μm to about 1,000 μm).

In some embodiments, the user signal and/or the reference signal can comprise an infrared wavelength or range thereof. The infrared wavelength can be selected from SWIR, MWIR, and/or NWIR. The infrared wavelength can comprise at least or at most about 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. The infrared wavelength can range between about 1 μm to about 15 μm, between about 1 μm to about 3 μm, X about 3 μm to about 8 μm, or between about 1 μm to about 15 μm.

In some embodiments, the at least one sensor can comprise an electromagnetic sensor. In some embodiments, the at least one sensor can comprise an infrared sensor. The infrared sensor can be configured to detect infrared signal mitted by a portion of a body of subject. Alternatively, the infrared sensor can be configured to (i) emit or send an initial light signal (e.g., infrared light signal) towards the subject and (ii) detect at least a portion of the initial light signal that is reflected by at least a portion of the body of the subject. In some embodiments, the at least one sensor can comprise a visible light sensor.

In some embodiments, the at least one radiation source (e.g., an optical source such as a lamp, light bulb, light emitting diode (LED), etc.) of the reference device can output (e.g., emit) a reference electromagnetic signal comprising an electromagnetic wavelength or wavelength range as provided herein.

In some embodiments, the user signal and/or the reference signal can comprise an acoustic or ultrasound wavelength or wavelength range thereof. The acoustic or ultrasound wavelength can comprise at least or at most about 100 nanometers (nm), 200 nm, 500 nm, 1 micrometer (μm), 2 μm, 5 μm, 10 μm, 20 μm, 50 μm, 100 μm, 200 μm, 500 μm, 1 millimeter (mm), 2 mm, 5 mm, 10 mm, 20 mm, 50 mm, 100 mm, 200 mm, 500 mm, 1 meter (m), 2 m, 5 m, 10 m, or 20 m. In some embodiments, the user signal and/or the reference signal can comprise an acoustic or ultrasound frequency or frequency range thereof. The acoustic or ultrasound frequency can comprise at least or at most about 20 Hertz (Hz), 50 Hz, 100 Hz, 200 Hz, 500 Hz, 1 kilohertz (kHz), 2 kHz, 5 kHz, 10 kHz, 20 kHz, 50 kHz, 100 kHz, 200 kHz, 500 kHz, 1 megahertz (MHz), 2 MHz, 5 MHz, 10 MHz, 20 MHz, 50 mHz, 100 MHz, or 200 MHz. For example, the acoustic or ultrasound frequency can range between about 0.1 MHz to about 100 MHz, between about 0.5 MHz to about 100 MHz, or between about 1 MHz to about 50 MHz (e.g., for ultrasound detection). In another example, the acoustic or ultrasound frequency can range between about 1 Hz to about 1 kHz, between about 10 Hz to about 1 kHz, between about 10 Hz to about 500 Hz, between about 10 Hz to about 300 Hz, or between about 100 Hz to about 1 kHz (e.g., for acoustic or audio detection).

In some embodiments, the at least one sensor can comprise an acoustic sensor (e.g., a microphone). In some embodiments, the at least one sensor can comprise an ultrasound sensor.

In some embodiments, the system as provided herein can comprise a photoacoustic sensor to detect user signal comprising photoacoustic waves, e.g., photoacoustic tomography. For example, acoustic and/or pressure waves generated by light absorption by at least a portion of the body of the subject and propagated to the surface of the body of the subject can be detected by the sensing device as provided herein, to monitor or determine the physiological feature of the subject.

In some embodiments, the at least one radiation source (e.g., a speaker) of the reference device can output (e.g., emit) a reference acoustic or ultrasound signal comprising an acoustic or ultrasound wavelength or wavelength range (or frequency or frequency range) as provided herein.

In some embodiments, the sensing device and the reference device can be disposed relative to each other such that the reference signal (e.g., the reference electromagnetic signal) is outputted to or within a detection field (e.g., an optical field of view, an acoustic or ultrasound detection field) of the at least one sensor. For example, at least a portion of the sensing device can be disposed to face at least a portion of the reference device. In operation, a distance between the sensing device and the reference device can be at least or at most about 0.1 meter (m), 0.2 m, 0.3 m, 0.4 m, 0.5 m, 0.6 m, 0.7 m, 0.8 m, 0.9 m, 1 m, 1.1 m, 1.2 m, 1.3 m, 1.4 m, 1.5 m, 1.6 m, 1.7 m, 1.8 m, 1.9 m, 2 m, 2.5 m, 3 m, 3.5 m, 4 m, 4.5 m, 5 m, 6 m, 7 m, 8 m, 9 m, or 10 m.

In some embodiments, the detection field of the at least one sensor can have an angle of detection of at least or at most about 30 degrees, 30 degrees, 90 degrees, 120 degrees, 150 degrees, 180 degrees, 210 degrees, 240 degrees, 270 degrees, 300 degrees, 330 degrees, 345 degrees, 350 degrees, 355 degrees, 356 degrees, 357 degrees, 358 degrees, 359 degrees, or 360 degrees.

In some embodiments, the at least one sensor of the sensing device can be configured to detect the reference signal from the reference device, to determine the location and/or distance of the reference device relative to the sensing device. Such determined location and/or distance of the reference device can be utilized in determining or monitoring the physiological feature of the subject.

In some embodiments, the sensing device and the reference device can be coupled (e.g., physically) to one another. In some embodiments, the sensing device and the reference device can be part of a same device. In some embodiments, the sensing device and the reference device may not be part of the same device. Whether part of the same device or of different devices, the sensing device and the reference device can be movable relative to one another, e.g., to calibrate the at least one sensor of the sensing device.

In some embodiments, the reference signal can comprise a signal (e.g., a wavelength) that is similar to that of an expected value of the user signal. In some embodiments, the user signal can be or can be indicative of heat signature of the subject. The reference signal can comprise an electromagnetic radiation wavelength or range thereof that is near or approximately the same as a human body temperature, such as at least or at most about 10 degrees Celsius, 11 degrees Celsius, 12 degrees Celsius, 13 degrees Celsius, 14 degrees Celsius, 15 degrees Celsius, 16 degrees Celsius, 17 degrees Celsius, 18 degrees Celsius, 19 degrees Celsius, 20 degrees Celsius, 21 degrees Celsius, 22 degrees Celsius, 23 degrees Celsius, 24 degrees Celsius, 25 degrees Celsius, 26 degrees Celsius, 27 degrees Celsius, 28 degrees Celsius, 29 degrees Celsius, 30 degrees Celsius, 31 degrees Celsius, 32 degrees Celsius, 33 degrees Celsius, 34 degrees Celsius, 35 degrees Celsius, 36 degrees Celsius, 37 degrees Celsius, 38 degrees Celsius, 39 degrees Celsius, 40 degrees Celsius, 45 degrees Celsius, or 50 degrees Celsius.

In some embodiments, the reference device can be configured to provide the reference signal at a fixed or predetermined reference signal value (e.g., a fixed or predetermined reference electromagnetic radiation signal value). In some embodiments, the reference device can be configured to provide a plurality of different reference signal values at once or at different times. The difference between a first refence signal value and a second reference signal value can be at least or at most about 1%, 5%, 10%, 15%, 20%, 30%, 40%, 50%, 60%, 70%, 80%, 90%, 95%, 100%, 120%, 150%, 200%, 300%, 400%, 500%, 600%, 700%, 800%, 900%, or 1,000%. In some embodiments, the value of the reference signal value can be changed, e.g., during operation of the system, based at least in part on the user signal (e.g., user electromagnetic radiation signal) detected by the at least one sensor. For example, the user signal value and the reference signal value can be indicative of a temperature of the subject (e.g., infrared wavelength or range thereof). When the body temperature of the subject (e.g., on average) is greater than a control value (e.g., an expected value obtained from the subject's past data, obtained from a cohort's data, or artificially generated or predetermined), the reference device can be configured to increase the reference signal value, to maintain or increase resolution of the user signal sensing and analysis thereof.

In some embodiments, the at least one sensor of the sensing device can be configured to determine the location and/or distance of the reference device relative to the sensing device, e.g., based at least in part on the detected user signal (e.g., user electromagnetic radiation signal). In some cases, a morphological feature of the at least the portion of the body of the subject can be determined or identified based on the detected user signal, and the morphological feature can be utilized to determine the relative distance between the subject at the at least one sensor. The morphological feature can be one-dimensional, two-dimensional, or three-dimensional. The morphological feature can be a dimension (e.g., diagonal dimension such as a diameter, circumference, etc.) or a shape of the at least the portion of the body, such as the subject's face. For example, thermal imaging data (or thermograph data) obtained by the at least one sensor can be used to identify a face or facial feature of the user (e.g., based on one or more facial recognition algorithms such as, but not limited to, Fisherfaces, FaceNet, Convolutional neural network, Multi-Task Cascaded Convolutional Neural Network (MTCNN), DeepFace, Eigenface, etc.) to estimate distance from the at least one sensor (e.g., thermal camera such as an infrared camera) to the user/subject.

In some embodiments, (i) historical data of the user signal (e.g., the user electromagnetic radiation signal) from the past can be retrieved from a database and (ii) the physiological feature of the user/subject can be determined based at least in part on a comparison between the historical data and the user signal that is currently detected or measured (e.g., during current use of the article of furniture by the user). In some embodiments, the historical data or analysis thereof can be utilized to generate a baseline (or a threshold value) indicating a normal or expected value or range of the user signal, and a predetermined difference in value between the current user signal and the base line (e.g., at least or at most about 1%, 5%, 10%, 15%, 20%, 30%, 40%, 50%, 60%, 70%, 80%, 90%, 95%, 100%, 120%, 150%, 200%, 300%, 400%, 500%, 600%, 700%, 800%, 900%, or 1,000%) can be utilized as an indication of an abnormal condition (e.g., an acute condition, a disease, etc.) of the subject. The historical data of the subject can comprise data collected by the sensing device or a different sensing device from a time period that is at least or at most about 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, 15 months, 18 months, 21 months, or 24 months prior to the current use of the article of furniture by the subject.

In some embodiments, the detected user signal (e.g., with or without the detected reference signal) can be utilized or analyzed to determine a biological signal of the subject as provided herein. The biological signal can be further analyzed to determine the physiological feature of the subject (e.g., disease).

In some embodiments, the at least one sensor (e.g., optical sensor, acoustic or ultrasound sensor) as provided herein can be referred to as a non-contact sensor, and the user signal (e.g., electromagnetic radiation, acoustic or ultrasound signal) detected by the non-contact sensor can be referred to as a non-contact sensing signal. In some embodiments, the system can further comprise an additional sensing device comprising at least one contact sensor (e.g., one or more sensors coupled to the article of furniture). The at least one contact sensor can be configured to detect a contact sensor signal (e.g., pressure signal, heart signal, breathing signal, motion signal, neurological signal, etc.) when in direct or indirect contact with the subject. In some cases, the physiological feature of the subject can be determined based on processing of the contact sensor signal and the non-contact sensing signal. As provided herein, non-limiting examples of the contact sensor can include a capacitance sensor, a pressure sensor such as a piezoelectric sensor, and a temperature sensor. In some embodiments, the at least one contact sensor is not a wearable sensor (e.g., not a smart watch).

In some embodiments, (i) a first heart signal data associated with plethysmograph (PG) signal of the subject can be determined based on the non-contact sensor signal (e.g., the user electromagnetic radiation signal), (ii) a second heart signal data associated with electrocardiograph (ECG) signal of the subject can be determined based on the contact sensor signal (e.g., pressure or piezoelectric data), and (iii) a pulse transit time (PTT) of the subject can be determined based on the first heart signal data and the second heart signal data. The PPT information can be usable for determining the physiological feature of the subject. In some cases, aortic pulse wave velocity (APWV) information of the subject can be determined based on the PTT information, and the APWV information can be usable for determining the physiological feature of the subject (e.g., arterial wall stiffness).

In some embodiments, the physiological feature as provided herein can be or can be indicative of a heart condition or a vascular disease, such as, for example, blood pressure, arterial wall stiffness, arrhythmia such as atrial fibrillation (AFib), nephropathy, retinopathy, neuropathy, tissue ischaemia, diabetic foot, dyslipidemia, arteriosclerosis, myocardial infarction, acute coronary syndrome, unstable angina pectoris, stable angina pectoris, stroke, peripheral arterial occlusive disease, cardiomyopathy, heart failure, heart rhythm disorders, vascular restenosis.

In some embodiments, the user signal (with or without the reference signal or the non-contact sensing signal as provided herein) can be utilized to monitor blood pressure of the subject, e.g., monitor if the blood pressure is going down over night while the subject is sleeping. When the monitored blood pressure increases over night (e.g., above a threshold limit or amount), such increase can be indication of a negative health condition of the subject, which can be flagged (e.g., digitally in a database) and shared to the user via a graphical user interface on a user device.

some embodiments, the physiological feature as provided herein can be or can be indicative of a neurological disease and/or a facial disorder (e.g., facial nerve disorder) of the subject. For example, the user signal (with or without the reference signal or the non-contact sensing signal as provided herein) can be utilized to identify symmetry or asymmetry of facial features or shapes of the subject (e.g., drooping) to recognize a neurological disease of the user.

In some embodiments, the physiological feature as provided herein can be or can be indicative of a sleep phase of the subject. The subject can be sleeping or suspected of sleeping when the user signal from the subject is detected by the sensing device as provided herein.

In some embodiments, the at least one sensor can comprise a non-visible light sensor (e.g., an infrared light sensor). The at least one sensor can further comprise a visible-light sensor configured to detect a visible light signal (e.g., having a wavelength or range thereof between about 300 nanometers to about 700 nanometers) associated with the user or the user's environment. The visible light signal from the visible light sensor and the user electromagnetic radiation signal (e.g., user infrared signal) can be analyzed (e.g., aligned or overlayed relative to one another) to generate a multi-modal imaging data that is usable for determining the physiological feature of the subject, as provided herein. For example, the system can comprise a stereo camera comprising at least the non-visible light sensor and the visible light sensor. The non-visible light sensor and the visible light sensor can share a same/common optical field of view. Alternatively, the non-visible light sensor and the visible light sensor can have different and non-overlapping optical field of view.

In some embodiments, the physiological feature can be a gesture (or body movement) of the subject. The sensing device (e.g., comprising the non-contact sensor) can be utilized to detect the user signal and analyze the user signal to determine or identify a particular gesture of the subject. In some cases, the user can be provided with an instruction of predetermined gestures. Alternatively, the computer processor can train one or more classifiers (e.g., machine learning algorithms) to identify and distinguish different gestures from the subject based on the detected user signal. In some cases, each gesture of the subject can be associated with an operation of a device, such as the article of furniture (e.g., regulating temperature of the article of furniture, regulating shape such as incline or decline of at least a portion of the article of furniture) or other devices in the subject's environment as provided herein. The associations between the one or more gestures and the corresponding instruction(s) for the one or more devices can be stored in and retrievable from a database operatively coupled to the sensing unit and/or the article of furniture. In some embodiments, the at least one sensor can be an infrared sensor, and the infrared sensor can be configured to detect infrared heat signature from the subject to determine the subject's gesture even in the dark.

In some embodiments, the sensing device as provided herein can comprise a housing. A relative position (e.g., distance or angle) between the at least one sensor (e.g., an infrared sensor) and an additional portion of the housing (e.g., a base portion of the housing) can be adjustable, e.g., to adjust the height of the at least one sensor relative to the ground. In some cases, the height of the at least one sensor relative to the ground may not be adjustable (e.g., may be at a fixed height relative to the ground or relative to the base portion of the housing of the sensing device).

In some embodiments, the sensing device can be part of a fixture (e.g., a stand, a lamp, a post, etc.) configured to be in proximity to or adjacent to an article of furniture. Alternatively or in addition to, the system can comprise an instruction (e.g., a physical or digital instruction) for the subject to place the sensing device at certain position (e.g., distance or angle) relative to the article of furniture, or an instruction to optimize such distance between the sensing device and the article of furniture. The distance can be a horizontal distance and/or a vertical distance.

In some embodiments, the system as provided herein can be utilized for body scanning of a subject during the subject's sleep. Data comprising user signal detected during such sleep by at least the sensing device as provided herein can be utilized to monitor, analyze, predict, or determine one or more interventions (e.g., future interventions) to delay onset or progress of a condition of the subject. Non-limiting examples of the condition can include disease (or disorder), stress, and sleep phase.

Non-limiting examples of the disease (or disorder) can include fever, pains, the heart condition as provided herein, sleep disorder as provided herein, facial disorder (e.g., facial nerve disorder), acute injuries, chronic injuries, neurodegenerative diseases, chronic diseases, proliferative diseases, cardiovascular diseases, genetic diseases, inflammatory diseases, autoimmune diseases, neurological diseases, hematological diseases, painful conditions, psychiatric disorders, metabolic disorders, chronic diseases, cancers, aging, age-related diseases, and diseases affecting any tissue in a subject. For example, age-related conditions include, heart failure, stroke, heart disease, atherosclerosis, neurodegenerative diseases (e.g., Parkinson's disease and Alzheimer's disease), cognitive decline, memory loss, diabetes, osteoporosis, arthritis, muscle loss, hearing loss (partial or total), eye-related conditions (e.g., poor eye sight or retinal disease), glaucoma, a progeroid syndrome (e.g., Hutchinson-Gilford progeria syndrome), and cancer. In another example, an age-related condition is senescence. In another example, the condition is nerve damage, e.g., a damage in the central nervous system (CNS). The nerve damage can be peripheral nerve damage. Alternatively, the nerve damage is neurapraxia, axonotmesis, or neurotmesis.

Non-limiting examples of cancer can include acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewing's sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwannoma, skin cancer, spinal axis tumors, squamous cell carcinomas, stomach cancer, synovial sarcoma, testicular cancer, uterine cancer, and tumors and their metastases.

Non-limiting examples of a neurological disease can include Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, and chronic fatigue syndrome.

Non-limiting examples of pain can include fibromyalgia, chronic neuropathic pain, or peripheral neuropathic pain.

In some embodiments, the system as provided herein further comprises the article of furniture.

### System and Methods for User Interaction With an Article of Furniture

The present disclosure provides systems, devices, methods, and techniques configured for comprehensive sleep tracking, environmental monitoring, and/or interactive user interface projection.

In certain aspects, provided herein are systems and methods configured to provide numerous benefits, including comprehensive sleep tracking, environmental factor monitoring, interactive user control, and projection of data onto a surface. In some embodiments, the method can comprise capturing data of the user's sleep using one or more sensors; analyzing the data to evaluate sleep conditions or to identify one or more health risks associated with the user's sleep patterns; and representing the analyzed data or implement one or more functions. In some embodiments, the system can use artificial intelligence (AI) for data analysis wherein the system can comprise training one or more AI models/algorithms to identify one or more health risks associated with the user's sleep patterns. In some embodiments, the system can process the data to extract/identify one or more health risks associated with the user's sleep; identify and perform one or more actions to improve the user's sleep health in response to identifying the one or more health risks; generate and send an alert to the user related to the one or more health risks; and generate and display, on a graphical user interface (GUI) or projecting on a surface (e.g., the ceiling), a comprehensive analysis of the user's sleep patterns using the data from the one or more sensors.

In an aspect, the present disclosure provides a system for enhancing sleep of a user. In some embodiments, the system can comprise a sleep enhancement device comprising (i) a regulator and (ii) a bed device having an at least partially internal heating or cooling element. In some cases, the regulator can be configured to adjust at least one sleep-related parameter for the bed device, e.g., to enhance sleep of the user. In some embodiments, the system can further comprise a projector configured to project an optical pattern to a wall or a ceiling adjacent the bed device. In some cases, the optical pattern can provide at least one of (a) status information for the bed device, local environment, or both or (b) at least one control option to direct operation of the sleep enhancement device.

In some embodiments, the optical pattern can provide (a) the status information for the bed device, local environment, and/or both. In some embodiments, the optical pattern can provide (b) the at least one control option to direct operation of the sleep enhancement device. In some embodiments, the optical pattern can provide both (a) and (b).

In some embodiments, the projector can be of various types, such as, as non-limiting examples, a digital light processing (DLP) projector, a liquid crystal display (LCD) projector, a light-emitting diode (LED) projector, or a laser projector. The projector's function can be to project images, patterns, or light onto a surface. In some embodiments, the projector can project an optical pattern onto the ceiling or the walls of the room. The optical pattern can provide information about the status of the user, the bed device, the local environment, or a combination thereof. It can also project control options to direct the operation of the sleep enhancement device. The projector can have various features to enhance its functionality. For example, it may have adjustable brightness levels to ensure that the projected image is visible but not overly bright. It may have a focus adjustment to ensure a sharp image regardless of the distance to the projection surface. It may have a wide-angle lens to cover a large area. It may also have a timer function to automatically turn off the projector after a certain period to conserve energy.

In some cases, the system can provide at least one control option that is selectable to adjust one or more parameters. The parameters can include the temperature of the bed device, bed device position, bed device orientation, a wake-up time setting of the sleep enhancement system, a time and date setting of the sleep enhancement device, a user or user profile information for the sleep enhancement device, or location information for the sleep enhancement device. The system can comprise a motion detector operatively coupled to the processor. The motion detector can be configured to detect movements by one or more users, one or more animals, or changes in the position or orientation of objects within the vicinity of the one or more users, and/or at least one gesture of the one or more users. In some embodiments, the motion detector can comprise one or more devices, such as one or more infrared (IR) sensors, one or more ultrasonic sensors, one or more optical sensors, one or more laser sensors, one or more microwave sensors, one or more tomographic motion detectors, or one or more video cameras. In some embodiments, the placement of the motion detector can be in various locations. At least a portion of the motion detector can be disposed within the bed device. Alternatively, or in addition to, at least a portion of the motion detector can be disposed outside of the bed device. For example, the at least the portion of the motion detector (e.g., some or entirety) can be disposed within a housing comprising at least a portion of the projector as provided herein, which housing can be configured to be disposed (e.g., installed) adjacent the bed device (e.g., mounted on a wall or ceiling adjacent the bed device). In another example, the motion detector can be configured to be disposed or placed on a nightstand. In a different example, the motion detector can be part of (or incorporated into) a wearable device (e.g., a smart watch, smart ring, etc.) or a mobile device (e.g., a cellphone). The terms “housing” and “capsule” may be used interchangeably herein.

In some embodiments, the system can include a processor and computer memory coupled to the processor. The computer memory can contain instructions for the processor to select the at least one control option based on a detection of at least one gesture of the one or more users. In some embodiments, the control options of the sleep enhancement device may include adjustments to one or more of the temperature of the bed device, a position of the bed device, an orientation of the bed device, a wake-up time setting of the sleep enhancement system, a time and date setting of the sleep enhancement device, a user or user profile information for the sleep enhancement device, a location information for the sleep enhancement device. In some embodiments, the one or more users can set a wake up alarm on the system to gradually increase a light level in the room, adjust the temperature of the bed device, and/or activate a gentle vibration to wake them up. In some embodiments, the one or more users can set a sleep timer for the system to start preparing the bed device for sleep by decreasing the light level in the room or adjusting the temperature to a cool down or heat up the bed device until reaching to a predefined temperature. In some embodiments, the system can store and use user or user profile information to personalize the sleep experience. For example, the device can record and remember a user's preferred temperature settings for the bed device, ideal bed device position and orientation, and preferred wake-up times. In some embodiments, the system can store information about a user's typical sleep patterns, like the time they usually go to bed and wake up, or any mid-night waking periods. In some embodiments, Health-related information can be stored in the user profile, such as known medical conditions, allergies, or prescriptions which might affect their sleep. In some embodiments, the system can use collected information to adjust its functions, like not increasing the bed temperature for users who suffer from night sweats. In some embodiments, the system can allow for multiple user profiles which can be particularly useful for a bed device used by more than one person, allowing each user to have their own personalized settings. For example, each side of the bed can adjust its temperature based on the preferences of the person sleeping on that side. In some cases, the user profile information may also include the user's sleep goals, such as increasing total sleep time or improving sleep quality.

In some embodiments, an optical axis of the projector and a sensing axis of the motion detector can be the same when a part or the entirety of the motion detector is disposed within the housing of the projector, allowing the two axes to potentially align. In some embodiments, the optical axis of the projector and the sensing axis of the motion detector can vary when the motion detector is placed any location other than within the housing of the projector, either inside or outside the bed device, leading to non-alignment of the axes.

In some embodiments, a user's interaction with a projected pattern (e.g., GUI) can be detected by the motion detector. In some embodiments, the motion detector can be configured to detect at least one gesture of the user which can be interpreted as a command to the system. The gestures can be simple movements or more complex patterns that the user performs to interact with the sleep enhancement device or to adjust its settings. For example, a user might wave their hand over the motion detector (e.g., comprising a motion sensor) to increase or decrease the temperature of the bed device. A different gesture, such as a two-finger swipe, could adjust the bed device position or orientation. Alternatively, the user could use a specific gesture to set a wake-up time or to access their user profile information on the sleep enhancement device. The motion detector can be designed to recognize a wide range of gestures, providing a user-friendly, hands-free control and intuitive way for users to interact with the sleep enhancement device.

In some embodiments, as provided herein, the optical pattern projected by the system can comprise a graphical user interface (GUI). The GUI can provide a visual and interactive way for the user to select the at least one control option. The GUI can display various control options as icons, buttons, sliders, or other interactive elements. These can represent different parameters that the user can adjust, such as the temperature of the bed device, the bed device position, bed device orientation, or a wake-up time setting of the sleep enhancement system. The user can interact with the GUI by using gestures detected by the motion detector, or through other interaction methods such as a remote control, a mobile app, or voice commands. The GUI can respond to these interactions by visually indicating the selected options or the adjustments made, providing the user with real-time feedback on the changes made to the sleep enhancement device settings.

In some embodiments, users can configure projector's parameters. The projector's parameters, as non-limiting examples can include resolution, brightness, contrast ratio, throw ratio, filed of view (FOV), screen size (i.e., projection size), aspect ratio, projection distance, lens shift range, keystone correction range, color accuracy, input lag, and/or zoom ratio.

For example, the resolution can be configured to be at least or at most about 640×480 (VGA), at least or at most about 800×600 (SVGA), at least or at most about 1024×768 (XGA), at least or at most about 1280×720 (HD), at least or at most about 1280×1024 (SXGA), at least or at most about 1600×1200 (UXGA), at least or at most about 1920×1080 (FHD), at least or at most about 2560×1440 (QHD), at least or at most about 3840×2160 (4K UHD), at least or at most about 5120×2880 (5K), at least or at most about 7680×4320 (8K UHD).

For example, the brightness can be configured to be at least or at most about 50 lumens (pico projectors), at least or at most about 100 lumens, at least or at most about 200 lumens, at least or at most about 300 lumens, at least or at most about 400 lumens, at least or at most about 500 lumens (low-end portable projectors), at least or at most about 600 lumens, at least or at most about 700 lumens, at least or at most about 800 lumens, at least or at most about 900 lumens, at least or at most about 1000 lumens, at least or at most about 2000 lumens (standard home theater projectors), at least or at most about 3000 lumens, at least or at most about 4000 lumens, at least or at most about 5000 lumens (mid-range commercial projectors), at least or at most about 10,000 lumens, at least or at most about 20,000 lumens, at least or at most about 30,000 lumens, at least or at most about 40,000 lumens, or at least or at most about 50,000 lumens (high-end commercial projectors).

For example, the contrast ratio can be configured to be at least or at most about 1,000:1 (low-end projectors), at least or at most about 2,000:1, at least or at most about 5,000:1, at least or at most about 10,000:1, at least or at most about 20,000:1, at least or at most about 50,000:1, at least or at most about 100,000:1, at least or at most about 200,000:1, at least or at most about 300,000:1, at least or at most about 400,000:1, at least or at most about 500,000:1, at least or at most about 1,000,000:1 (high-end home theater projectors), at least or at most about 1,500,000:1, or up to at least or at most about 2,000,000:1 (high-end commercial projectors).

For example, the throw ratio can be configured to be at least or at most about 0.2:1 (ultra-short throw), at least or at most about 0.5:1, at least or at most about 1.0:1 (short throw), at least or at most about 1.5:1, at least or at most about 2.0:1 (standard throw), at least or at most about 2.5:1, up to at least or at most about 3.0:1 (long throw).

For example, the field of view (FOV) can be configured to be at least or at most about 5°, at least or at most about 10°, at least or at most about 20°, at least or at most about 30°, at least or at most about 40°, at least or at most about 50°, at least or at most about 60°, at least or at most about 70°, at least or at most about 80°, at least or at most about 90°, at least or at most about 100°, at least or at most about 110°, at least or at most about 120°, at least or at most about 130°, at least or at most about 140°, at least or at most about 150°, or at most about 160°.

For example, the screen size (or projection size) can be configured to be at least or at most about 10 inches diagonal, at least or at most about 20 inches, at least or at most about 30 inches, at least or at most about 40 inches, at least or at most about 50 inches, at least or at most about 60 inches, at least or at most about 70 inches, at least or at most about 80 inches, at least or at most about 90 inches, at least or at most about 100 inches, at least or at most about 200 inches, at least or at most about 300 inches, at least or at most about 400 inches, or at least or at most about 500 inches diagonal.

For example, the aspect ratio can be configured to be at least or at most about 4:3, at least or at most about 16:9 (widescreen), at least or at most about 16:10, at least or at most about 1.85:1 (American widescreen cinema), at least or at most about 2.35:1 (CinemaScope), or at least or at most about 21:9 (widescreen cinematic).

For example, the projection distance can be configured to be at least or at most about 0.1 meters, at least or at most about 0.2 meters, at least or at most about 0.3 meters, at least or at most about 0.4 meters, at least or at most about 0.5 meters, at least or at most about 0.6 meters, at least or at most about 0.7 meters, at least or at most about 0.8 meters, at least or at most about 0.9 meters, at least or at most about 1 meter, at least or at most about 2 meters, at least or at most about 3 meters, at least or at most about 4 meters, at least or at most about 5 meters, at least or at most about 6 meters, at least or at most about 7 meters, at least or at most about 8 meters, at least or at most about 9 meters, at least or at most about 10 meters, at least or at most about 20 meters, at least or at most about 30 meters, at least or at most about 40 meters, or at least or at most about 50 meters.

For example, the lens shift range can be configured to be at least or at most about ±5%, at least or at most about ±10%, at least or at most about ±15%, at least or at most about ±20%, at least or at most about ±25%, at least or at most about ±30%, at least or at most about ±35%, at least or at most about ±40%, at least or at most about +45%, at least or at most about ±50%, at least or at most about ±55%, at least or at most about ±60%, at least or at most about ±65%, at least or at most about ±70%, at least or at most about ±75%, at least or at most about ±80%, at least or at most about ±85%, at least or at most about ±90%, at least or at most about ±95%, or at least or at most about ±100%.

For example, the keystone correction range can be configured to be at least or at most about ±10°, at least or at most about ±20°, at least or at most about ±30°, at least or at most about ±40°, at least or at most about ±50°, or at least or at most about ±60°.

For example, the color accuracy can be configured to be at least or at most about 10%, at least or at most about 20%, at least or at most about 30%, at least or at most about 40%, at least or at most about 50%, at least or at most about 60%, at least or at most about 70%, at least or at most about 80%, at least or at most about 90%, or at least or at most about 100%.

For example, the input lag can be configured to be at least or at most about 5 ms, at least or at most about 10 ms, at least or at most about 15 ms, at least or at most about 20 ms, at least or at most about 25 ms, at least or at most about 30 ms, at least or at most about 35 ms, at least or at most about 40 ms, at least or at most about 45 ms, at least or at most about 50 ms, at least or at most about 55 ms, at least or at most about 60 ms, at least or at most about 65 ms, at least or at most about 70 ms, at least or at most about 75 ms, at least or at most about 80 ms, at least or at most about 85 ms, at least or at most about 90 ms, at least or at most about 95 ms, or at least or at most about 100 ms.

For example, the zoom ratio can be configured to be a fixed lens (no zoom), at least or at most about 1.0×, at least or at most about 2.0×, at least or at most about 3.0×, at least or at most about 4.0×, at least or at most about 5.0×, at least or at most about 6.0×, at least or at most about 7.0×, at least or at most about 8.0×, at least or at most about 9.0×, at least or at most about 10.0×, at least or at most about 15.0×, at least or at most about 20.0×, at least or at most about 25.0×, or at least or at most about 30.0× zoom.

In some embodiments, the system can further comprise a cooling unit that is operatively coupled to the projector for cooling (or lowering temperature of) at least a portion of the projector. Non-limiting examples of the cooling unit can include a cooling fan, a liquid cooling system, a heat sink, a thermoelectric cooler, a refrigeration system, an air conditioning unit, a heat pipe, a vapor chamber, a cooling pad, a phase change material, or a passive cooling system. The cooling unit can comprise at least or at most about 1 cooling unit, at least or at most about 2 cooling units, at least or at most about 3 cooling units, at least or at most about 4 cooling units, at least or at most about 5 cooling units, at least or at most about 6 cooling units, at least or at most about 7 cooling units, at least or at most about 8 cooling units, at least or at most about 9 cooling units, or at least or at most about 10 cooling units.

In some embodiments, the system can further comprise a cooling unit (e.g., cooling fan) that is operatively coupled to the projector. In some embodiments, the cooling unit can be disposed adjacent the projector. The cooling unit and the project may or may not be in physical contact with one another. The cooling unit can be configured to turn on (or be activated) based on one or more conditions, including, but not limited to, (i) when the projector has been operating for more than a predetermined threshold duration of time, (ii) when a temperature of at least a portion of the project reaches a predetermined temperature, (iii) when the projector is running a high-performance task (e.g., high-resolution or intensive display task), (iv) when an ambient temperature in a projector's location exceeds a predetermined temperature, (v) when the cooling unit itself reaches a predetermined temperature, (vi) when a projector's lamp usage exceeds a predetermined percentage, or (vii) when a power supply unit of the projector reports a power surge. The cooling unit can help to prevent overheating of the projector. The cooling unit can automatically activate based on the one or more conditions (e.g., once the operation time of the projector exceeds the predetermined threshold), ensuring the projector maintains a safe operating temperature and prolonging the lifespan of the projector. Once activated, the cooling can be configured to remain on (or in operation) for a predetermined period of time. Alternatively, the cooling unit can be configured to determine a period of time for the cooling unit to operate based on one or more conditions, including, but not limited to, how long the project has been operating prior to activation of the cooling unit, (ii) a temperature of the projector prior to or during the activation of the cooling unit, (iii) a duration of the high-performance task that the projector is running, (iv) the ambient temperature in the projector's location, (v) the temperature of the cooling unit itself, (vi) the percentage of lamp usage in the projector at the time of activation, or (vii) the status of the projector's power supply unit at the time of activation.. The cooling unit can be configured turn off (or be inactivated) based on ore more conditions, including, but not limited to, (i) a predetermined duration of operation, (ii) when the temperature of the projector has decreased to a predetermined temperature, (iii) when the high-performance task that the projector was running has ended, (iv) when the ambient temperature in the projector's location has fallen to a predetermined temperature, (v) when the temperature of the cooling unit itself has decreased to a predetermined temperature, (vi) when the lamp usage in the projector drops below a predetermined percentage, or (vii) when the power supply unit of the projector reports reaching to a predetermined power usage. Alternatively, or in addition to, the cooling unit can be configured such that the user can manually (e.g., via a physical button or via interaction with a remote, such as a user application on a user device) turn on and/or turn off the cooling unit or set a timer for the cooling unit. The cooling unit can comprise at least or at most about 1 cooling unit, at least or at most about 2 cooling units, at least or at most about 3 cooling units, at least or at most about 4 cooling units, at least or at most about 5 cooling units, at least or at most about 6 cooling units, at least or at most about 7 cooling units, at least or at most about 8 cooling units, at least or at most about 9 cooling units, or at least or at most about 10 cooling units.

In some embodiments, the predetermined threshold duration of time for determining when to turn on (or activate) of the cooling unit can be at least or at most about 1 second, at least or at most about 2 seconds, at least or at most about 3 seconds, at least or at most about 4 seconds, at least or at most about 5 seconds, about at least or at most about 6 seconds, at least or at most about 7 seconds, at least or at most about 8 seconds, at least or at most about 9 seconds, at least or at most about 10 seconds, at least or at most about 20 seconds, at least or at most about 30 seconds, at least or at most about 40 seconds, at least or at most about 50 seconds, at least or at most about 1 minute, at least or at most about 2 minutes, at least or at most about 3 minutes, at least or at most about 4 minutes, at least or at most about 5 minutes, at least or at most about 10 minutes, at least or at most about 20 minutes, at least or at most about 30 minutes, at least or at most about 40 minutes, at least or at most about 50 minutes, at least or at most about 1 hour, at least or at most about 2 hours, at least or at most about 3 hours, at least or at most about 4 hours, at least or at most about 5 hours, or at least or at most about 6 hours.

In some embodiments, the predetermined threshold temperature for determining when to turn on (or activate) of the cooling unit can be at least or at most about 1 degrees Celsius, at least or at most about 2 degrees Celsius, at least or at most about 3 degrees Celsius, at least or at most about 4 degrees Celsius, at least or at most about 5 degrees Celsius, at least or at most about 10 degrees Celsius, at least or at most about 15 degrees Celsius, at least or at most about 20 degrees Celsius, at least or at most about 30 degrees Celsius, at least or at most about 40 degrees Celsius, at least or at most about 50 degrees Celsius, at least or at most about 60 degrees Celsius, at least or at most about 70 degrees Celsius, at least or at most about 80 degrees Celsius, or at least or at most about 90 degrees Celsius.

In some embodiments, the predetermined duration of operation of the cooling unit (e.g., upon activation of the cooling) can be at least or at most about 5 seconds, at least or at most about 10 seconds, at least or at most about 20 seconds, at least or at most about 30 seconds, at least or at most about 40 seconds, at least or at most about 50 seconds, at least or at most about 1 minute, at least or at most about 2 minutes, at least or at most about 3 minutes, at least or at most about 4 minutes, at least or at most about 5 minutes, at least or at most about 10 minutes, at least or at most about 20 minutes, at least or at most about 30 minutes, at least or at most about 40 minutes, at least or at most about 50 minutes, at least or at most about 1 hour, at least or at most about 2 hours, at least or at most about 3 hours, at least or at most about 4 hours, at least or at most about 5 hours, or at least or at most about 6 hours.

In some embodiments, a noise level of the cooling unit, often measured in decibels (dB), may vary based on the specific type of cooling unit used. For example, the noise level can be at least or at most about 10 dB, at least or at most about 15 dB, at least or at most about 20 dB, at least or at most about 25 dB, at least or at most about 30 dB, at least or at most about 40 dB, or at least or at most about 50 dB. The noise level may be within a certain range to ensure it does not disrupt the viewing experience. To control the noise level, the cooling unit may be selected with the noise level within a predefined range that does not disturb the user's viewing experience. Alternatively, or in addition to, noise-reducing technologies can be implemented to alleviate or reduce the noise level generated by the cooling unit. These technologies may include acoustic damping materials, sound barriers, sound absorbers, active noise cancellation, vibration isolation, or specific design features such as fan blade shape and orientation that produce less noise. Alternatively, or in addition to, performance time (or usage time) of the cooling unit can be controlled, as provided herein, to reduce or control an average noise level of the cooling unit throughout its usage (e.g., throughout a user's sleep time).

In some embodiments, a bed device can be an article of furniture used for sleep or rest. The bed device can comprise a mattress, a mattress pad, a mattress cover, a bed sheet, a pillow, and/or a covering (e.g., a blanket). One or more users can sleep or rest on and/or adjacent to a surface of the bed device. The surface can be a top surface of the bed device. The top surface of the bed device can be flat or textured. The bed device can be the mattress. The bed device can be the mattress pad that covers at least a portion of a surface of a mattress or at least a surface of the mattress. Alternatively, or in addition to, the user(s) can sleep under a surface of the bed device. The surface can be one or more surfaces of a covering, such as, for example, a blanket. The blanket can be disposed on top of at least a part of the user(s). The bed device can be the blanket.

In some embodiments, the bed device can comprise a mattress. The mattress can be of various types, as non-limiting examples, such as innerspring, memory foam, latex, hybrid, or airbed. It can be in multiple sizes like twin, full, queen, or king. The mattress materials can range, as non-limiting examples, from memory foam to latex, wool, cotton, or a combination of thereof. In some embodiments, the mattress can comprise at least a partially internal cooling or heating element. The internal cooling or heating element can be, as non-limiting examples, a thermal pad, a temperature-controlled liquid (e.g., water and/or oil) or gas (e.g., air) circulation system, or an electric heating pad. The element can comprise one or more embedded channels or tubes. The materials used for the internal cooling or heating element can be made, as non-limiting examples, from polymers, rubber, metal, composites or any combination thereof. The temperature-controlled liquid or gas circulation system can circulate within these channels or tubes. The embedded channels or tubes can either cover a portion of the mattress or span across the entire mattress. In some embodiments, the heating or cooling element can comprise one or more sets of the embedded channels or tubes. Each set can be allocated to different parts of the mattress, allowing for individual temperature control which can be particularly useful for multiple users. For instance, in a scenario where two users are sharing the same mattress, one user can adjust their side of the bed for cooling while the other user can adjust theirs for heating. The internal cooling or heating element can work by either increasing or decreasing the temperature of the mattress to offer a comfortable sleeping environment. They can feature various settings to adjust the intensity of cooling or heating, timers, remote control operation, and energy-saving modes.

In some embodiments, the bed device can comprise a mattress cover. The mattress cover can be of various types, as non-limiting examples, such as fitted, zippered, or elastic strap. It can be in multiple sizes like twin, full, queen, or king to match the mattress sizes. The mattress cover materials can range, as non-limiting examples, from cotton, polyester, down, wool, or a combination thereof. In some embodiments, the mattress cover can comprise at least a partially internal cooling or heating element. The internal cooling or heating element can be, as non-limiting examples, a thermal pad, a temperature-controlled liquid or gas circulation system, or an electric heating pad. The element can comprise one or more embedded channels or tubes. The materials used for the internal cooling or heating element can be made, as non-limiting examples, from polymers, rubber, metal, composites, or any combination thereof. The temperature-controlled liquid or gas circulation system can circulate within these channels or tubes. The embedded channels or tubes can either cover a portion of the mattress cover or span across the entire mattress cover. In some embodiments, the heating or cooling element can comprise one or more sets of the embedded channels or tubes. Each set can be allocated to different parts of the mattress cover, allowing for individual temperature control which can be particularly useful for multiple users. For instance, in a scenario where two users are sharing the same bed, one user can adjust their side of the bed for cooling while the other user can adjust theirs for heating. The internal cooling or heating element can work by either increasing or decreasing the temperature of the mattress cover to offer a comfortable sleeping environment. They can feature various settings to adjust the intensity of cooling or heating, timers, remote control operation, and energy-saving modes.

In some embodiments, the sleep enhancement device can comprise a hub (or a console, as used interchangeably herein). The hub can be a device that includes processor that receives the various data disclosed herein such as the temperature, biological signals, and other types of information regarding the user's sleep and generates temperature adjustment for the bed device. This can cause the bed device to heat or cool, improving the sleep experience for the user. The hub can generate temperature adjustments that can allow for the bed device to change the temperature in response to recorded and/or configured conditions.

The console can be of various types and can be a standalone device, a wall-mounted device, or a device integrated into another piece of furniture or technology, as non-limiting examples. For example, the console and the at least partially internal cooling or heating element of the bed device may be separate units that are connected (e.g., digitally, physically, electrically, etc.) to one another. In another example, the console and the at least partially internal cooling or heating element of the bed device may be part of a single unit (e.g., contained within a common housing unit). The console can comprise various components such as a display screen, buttons, dials, or touch-sensitive surfaces for controlling its functions. The console can perform various functions, such as controlling the temperature, setting timers, adjusting the intensity of cooling or heating, and providing status updates on the bed device. In some embodiments, the console can comprise a temperature regulator. The temperature regulator can be, as non-limiting examples, a mechanical thermostat, an electronic thermostat, or a smart thermostat. In some cases, the temperature regulator can achieve temperature regulation via thermoelectric temperature regulation. The temperature regulator can be coupled to or can comprise a thermoelectric temperature regulator. Thermoelectric temperature regulation (e.g., heating and/or cooling) can be implemented using an electric-based system (e.g., by a thermoelectric engine). The thermoelectric engine can be configured to convert electrical energy into a heat flux (or a temperature difference) or convert the heat flux into electrical energy. The thermoelectric engine can be a solid-state device. In some examples, the temperature regulator can be a thermoelectric cooler, a thermoelectric heat pump, and/or a Peltier device. The temperature regulator can be operatively coupled to the at least partially internal cooling or heating element of the bed device. The operative connection allows the temperature regulator to control the temperature of the internal cooling or heating element. The temperature regulator can adjust the temperature to a chosen level, maintain a constant temperature, or change the temperature at set intervals. The temperature regulator can be manually operated, remotely controlled, or automatically adjusted based on pre-set preferences or environmental conditions.

In some embodiments, the at least partially internal heating or cooling element can comprise one or more fluid channels. These fluid channels can be positioned at least partially within a body of the bed device. The fluid channels can vary in number, size, and arrangement depending on the design of the bed device. They can be as non-limiting examples, narrow tubes, wide ducts, or micro-channels. The fluid channels can be made from materials such as, as non-limiting examples, polymers, rubber, metal, composites, or any combination thereof. The fluid channels can be positioned to maximize the efficiency of heating or cooling, such as near the surface of the bed device or in areas that typically come into contact with the user. The fluid in the channels can be a heat transfer fluid, which can be, as non-limiting examples, water, refrigerant, or a thermally conductive oil. The fluid channels can allow the fluid to circulate, transferring heat to or from the user to adjust the temperature of the bed device. The fluid circulation can be controlled by the temperature regulator in the console to, for example, maintain a comfortable sleeping environment or wake up an individual from sleep. In some embodiments, the portion of the heating or cooling element that is at least partially internal (or integrated within) the bed device may not be visible from an outer surface of the bed device.

In some embodiments, the at least one sleep-related parameter that is adjusted by the regulator can comprise a temperature of the bed device, a temperature of the user, or both. In some embodiments, the at least one sleep-related parameter can comprise the temperature of the bed device. In some embodiments, the at least one sleep-related parameter can comprise the temperature of the user. In some embodiments, the at least one sleep-related parameter can comprise the temperature of the bed device and the temperature of the user. In some cases, a single user may be using the bed device. In some cases, one or more users may be using the bed device (e.g., sleeping on different zones of the bed device), and the at least one sleep-related parameter can comprise a first temperature of a first user and a second temperature of a second user, and the first temperature and the second temperature may be the same or different. In some embodiments, the at least one sleep-related parameter can comprise the temperature of different zones of the bed device. These different zones can be designated for a single user or multiple users. In some cases, a single user may be using the bed device and may wish to have different temperatures in different zones. For example, the user may prefer a cooler head region and a warmer foot region. In this instance, the at least one sleep-related parameter can comprise a first temperature of a first zone and a second temperature of a second zone, and the first temperature and the second temperature may be the same or different. In some cases, one or more users may be using the bed device, each occupying a different zone. For instance, one user might prefer a cooler temperature while the other user might prefer a warmer temperature. In this scenario, the at least one sleep-related parameter can comprise a first temperature of a first user's zone and a second temperature of a second user's zone, and the first temperature and the second temperature may be the same or different.

In some embodiments, the sleep enhancement device can comprise a sensor that is coupled to the bed device. The sensor coupled to the bed device can be configured to detect a parameter of the bed device, the user, or both. The sensor coupled to the bed device can be part of the bed device, part of a room or space comprising the bed device there within, and/or a part of a wearable device that the user of the bed device is wearing. The sensor(s) can be configured to measure one or more biological signals comprising heart signal, breathing signal, respiration rate, temperature, movement, presence, etc. Non-limiting examples of a sensor can include a capacitance sensor (e.g., for detecting presence), a piezo sensor (e.g., for measuring a biological signal such as heart signal, breathing signal, respiration signal, etc.), a temperature sensor (e.g., for measuring temperature of the user, or that of the bed), infrared sensor, pressure sensor, optical sensor, and/or humidity sensors. The sensor can comprise one or more sensors, such as at least or at most about 1 sensors, such as at least or at most about 2 sensors, at least or at most about 3 sensors, at least or at most about 4 sensors, at least or at most about 5 sensors, at least or at most about 6 sensors, at least or at most about 7 sensors, at least or at most about 8 sensors, at least or at most about 9 sensors, or at least or at most about 10 sensors. The one or more sensors can comprise same types of sensors or different types of sensors.

In some embodiments, the status information projected by the system may include one or more of the present time, a temperature of the user, a temperature of the bed device, a temperature of an environment adjacent the bed device, a humidity of the user, a humidity of the bed device, a humidity of an environment adjacent the bed device, a movement level of the user, a sleep phase of the user, a body posture of the user, an arterial stiffness of the user, a fever of the user, a menstrual cycle status of the user, a bodily injury of the user, or an energy level of the sleep enhancement device. In some embodiments, the sensor can be configured to detect various parameters of the user. These parameters can include, as non-limiting examples, the temperature of the user, which can be measured using direct-body contact temperature sensors, remote temperature sensors, or a combination thereof. The direct-body contact sensors can be embedded in the bed device, placed on a user's skin, or integrated into wearable technology such as smartwatches or fitness trackers.

In some embodiments, the remote temperature sensors can be placed at a distance without a direct contact with a user's body. Some non-limiting examples of direct-body contact and remote temperature sensors may include thermocouples, resistance temperature detectors (RTDs), infrared thermometers, thermistors, or temperature-sensitive tapes or other temperature-sensing devices; a humidity of the user, which could refer to the measurement of perspiration or sweat levels using humidity sensors such as capacitive humidity sensors, resistive humidity sensors, thermal conductivity humidity sensors or other humidity-sensing devices; the movement level of the user, which can be detected using motion sensors, accelerometers, gyroscopes, passive infrared (PIR), or other movement detection sensors; the sleep phase of the user, which can be determined through a combination of movement data (e.g., electromyogram (EMG) sensors for muscle activity monitoring), eye movement tracking (e.g., electrooculogram (EOG) sensors for eye movement tracking), brainwave monitoring (e.g., electroencephalogram (EEG) sensors for brainwave monitoring), or other sleep phase detection sensors; the body posture of the user, which can be detected using pressure sensors, depth cameras, pressure mats, wearable posture trackers or other posture-tracking technologies; a facial skin mole of the user, which can be detected using high-resolution imaging technology, dermatoscopes or other sensors used for monitoring skin conditions; arterial stiffness of the user, which can be measured using pulse wave velocity sensors, tonometers, or other cardiovascular monitoring devices; fever of the user, which can be detected by monitoring the user's temperature over time using temperature sensors such as thermometers, ear thermometers, infrared thermometers, or other temperature-sensing devices; the menstrual cycle of the user, which can be determined based on body temperature changes, hormone level testing kits, or period tracking through user input/period tracking apps; and a bodily injury of the user, which can be detected by monitoring changes in movement, temperature, pain assessment tools, medical imaging devices or other bodily injury detection technologies; a Heart Rate Variability (HRV) of the user, which is a measure of the variation in the time interval between heartbeats and can be measured using ECG sensors or PPG sensors; a respiratory rate of the user, which can be measured using chest straps, wearable devices, or microphones that detect the sound of breathing; a Blood Oxygen Saturation (SpO2) of the user, which is a measure of how much oxygen is being carried by the hemoglobin in the blood and can be measured using pulse oximeters; a Galvanic Skin Response (GSR) of the user, which measures the electrical conductance of the skin; a Electrodermal Activity (EDA) of the user, which is a measure of the electrical conductance of the skin and can be used to understand the physiological or psychological state of a person; a pupil dilation of the user, which can be measured using eye-tracking cameras; a Electrocardiogramaphy (ECG) of the user, which measures the electrical activity of the heart and can be used to identify a range of heart conditions; and a Electromyography (EMG) of the user, which measures the electrical activity of muscles and can be used to detect neuromuscular diseases, nerve compression or injury, and other conditions.

In some embodiments, the sensor can be configured to detect various parameters of the bed device. These parameters can include, as non-limiting examples, the temperature of the bed device, which can be measured using temperature sensors such as thermocouples, resistance temperature detectors (RTDs), infrared thermometers, or other temperature-sensing devices; a temperature of an environment adjacent the bed device, which can be measured using ambient temperature sensors or infrared thermometers that can detect the temperature of the surrounding air or surfaces; a humidity of the bed device, which can be measured using humidity sensors such as capacitive humidity sensors, resistive humidity sensors, thermal conductivity humidity sensors, or other humidity-sensing devices; or the humidity of an environment adjacent the bed device, which can be measured using ambient humidity sensors or hygrometers.

In some embodiments, the sensor coupled to the bed device can be operatively coupled to the regulator. This operative coupling allows the sensor to transmit the detected parameter data to the regulator. The regulator can then use this data to adjust the at least one sleep-related parameter for the bed device. The regulator can be configured to adjust the at least one sleep-related parameter based on the detected parameter. In some cases, when the sensor can detect that the temperature of the bed device or the user is different as compared to a control value (e.g., from historical data, from a cohort study, from different time points of the user's use of the bed device, etc.), the regulator can adjust the at least partially internal cooling or heating element to adjust the temperature. For example, when the sensor detects that the temperature of the bed device or the user exceeds a predefined limit, the regulator can adjust the internal cooling or heating element to bring the temperature back within the predefined limit, or to another range as configured. Similarly, when the sensor detects movement from the user indicating discomfort, the regulator can adjust the at least one sleep-related parameter accordingly to enhance the user's comfort.

In some embodiments, the regulator can be configured to adjust at least one sleep-related parameter, such as the temperature of the bed device, based on the presence or absence of the user on the bed device. For example, when the user is present on the bed device, the regulator can adjust the temperature of the internal heating or cooling element to create a comfortable sleep environment. When the user is feeling cold, the regulator can increase the temperature to provide warmth. Alternatively, when the user is feeling hot or when the ambient temperature is high, the regulator can activate the cooling element to reduce the temperature of the bed device, making it more comfortable for the user. Additionally, for medical reasons such as pain alleviation, the regulator can adjust the temperature to provide thermal or cold therapy as needed. In the absence of the user, the regulator can prepare the bed device for the user's return by adjusting the temperature to fall within a configured range or can lower the temperature of the heating or cooling element to conserve energy.

In some embodiments, the bed device can be configured to adjust the bed device position, bed device orientation, or both based on the user's presence on the bed device. Adjustments to the bed device position can be made using various mechanisms such as motor-driven actuators, hydraulic systems, pneumatic devices, or a combination thereof. The mechanisms can shift the bed device to different locations, or adjust its height, according to the user's comfort or specific needs. Changes to the bed device orientation can be accomplished using adjustable joints, hinges, or spring mechanisms that allow for tilting or swiveling of different parts of the bed device. For example, the head or foot of the bed device can be raised or lowered to enhance the user's comfort or accommodate medical requirements. The bed device can use data from sensors that detect the user's presence and position on the bed device to control the adjustments. Some non-limiting examples of the bed device position can include raising or lowering an entire bed device to adjust a height, shifting the bed device horizontally to adjust a location, or tilting the bed device laterally or longitudinally. Some non-limiting examples of the bed device orientation can include elevating or lowering a head of the bed device, raising, or dropping a foot of the bed device, adjusting an incline of a middle section of the bed device for ergonomic support.

In some embodiments, the system can include an adjustable mattress frame. The adjustable mattress frame can be configured to couple with a mattress and can have the capability to adjust a position, an orientation, or both of at least a portion of the mattress. The adjustable mattress frame can raise or lower different sections of the mattress, such as the head or foot, changing its orientation for user comfort. It may also slide or rotate the mattress, altering its position to best suit the user's needs. The adjustments can be made using various mechanisms such as motor-driven actuators, hydraulic or pneumatic systems, or spring mechanisms.

In some embodiments, the sensor can comprise an optical sensor configured to detect electromagnetic radiation (e.g., emitted or reflected) from a source, such as the bed device, the user, or both. In some embodiments, the sensor can comprise an infrared sensor. The infrared sensor can detect infrared reflectance signals or electromagnetic emittance signals from the bed device, the user, or both. Infrared reflectance signals are generated when infrared light, emitted by an infrared light source, bounces off an object and returns to the sensor. The amount of reflected light can provide information about the object's surface temperature. Electromagnetic emittance signals, on the other hand, are naturally emitted by all objects above absolute zero temperature. These signals can be detected without the need for an external light source. The intensity of the emittance signal can provide information about the object's temperature. In some embodiments, the one or more sensors may comprise video cameras, visible light cameras, night vision cameras, infrared cameras, thermal imaging cameras, or a combination thereof. In some embodiments, the infrared (IR) cameras can comprise short wavelength, mid-wavelength, long wavelength infrared cameras, or a combination thereof. IR cameras may be able to perform detection of heat emitting objects, such as human objects. In some embodiments, active illumination may be employed. Active illumination may refer to a method of lighting in which a source of light is part of a same system comprising cameras or imaging devices. The system may be able to generate a thermal image in complete darkness from an IR camera. IR images may be used in conjunction with or instead of visible spectra images. The IR imaging may be used for motion detection, facial recognition (e.g., unique identification), demographics recognition (e.g., gender, age, race, etc.), state recognition (e.g., emotional state, health state), character recognition, bar/QR code reading, object recognition, detection of humans or other live beings, movement detection, human or other motion trajectory detection, gesture recognition, and/or emotion recognition. In some embodiments, the system may comprise one or more IR illuminators. The one or more IR illuminators may contain arrays of IR LEDs, and may have an illumination range of at least or at most about 1-ft, at least or at most about 2-ft, at least or at most about 3-ft, at least or at most about 4-ft, at least or at most about 5-ft, at least or at most about 6-ft, at least or at most about 7-ft, at least or at most about 8-ft, at least or at most about 8-ft, at least or at most about 10-ft, at least or at most about 15-ft, at least or at most about 20-ft, at least or at most about 25-ft, at least or at most about 30-ft, at least or at most about 35-ft, at least or at most about 40-ft, at least or at most about 45-ft, or at least or at most about 50-ft. In some embodiments, alternatively, or in addition to the infrared sensors, other types of electromagnetic sensing can be used. These can include, as non-limiting examples, microwave sensors, which can detect movement by sensing changes in the microwave field; radio frequency sensors, which can detect proximity or movement by sensing changes in the radio frequency field; and visible light sensors, which can detect changes in light levels; ultraviolet (UV) sensors, which measure the intensity of UV radiation; terahertz sensors, which operate in the terahertz frequency range and can detect and identify a wide range of materials; X-ray sensors, which detect X-ray radiation; gamma ray sensors, which detect high-energy electromagnetic radiation; and magnetic field sensors, also known as magnetometers, which measure the strength and direction of magnetic fields.

In some embodiments, the sensor can comprise a light source operatively coupled to the optical sensor. The light source can be configured to direct light (e.g., one or more electromagnetic wavelengths) towards the bed device, the user, or both, such that the sensor can detect at least a portion of the directed light that is reflected by the bed device, the user, or both. In some embodiments, the optical source can comprise an infrared light source that is operatively coupled to the infrared sensor. The infrared light source can be configured to direct infrared light towards the bed device, the user, or both. The infrared light source can act as an infrared illumination source, casting infrared light onto the bed device or the user. This infrared light can then be reflected back to the infrared sensor, resulting in an infrared reflectance signal. This signal can provide information about the temperature of the bed device or the user, or other parameters. The infrared light source can be various types, as non-limiting examples, such as an infrared light-emitting diode (LED), an infrared laser, or an infrared lamp. The choice of infrared light source can depend on factors such as the required intensity of infrared light, the distance between the light source and the bed device or user, and the desired accuracy of the measurements.

In some embodiments, the system can further comprise a housing containing at least a portion of the projector and at least a portion of the sensor (e.g., the infrared sensor). The projector may be contained partially or entirely within the housing. The sensor may be contained partially or entirely within the housing. The housing can further contain at least a portion of (e.g., partially, or entirely) the optical source (e.g., the infrared light source). The housing can comprise an opening for the optical source. This opening allows the infrared light source to direct infrared light from inside the housing, through the opening, and towards the bed device or the user.

In some embodiments, the housing as provided herein can be a part of the sleep enhancement device. For example, the housing can be at least partially internal to or directly coupled to the bed device, such as the mattress, the mattress cover, a bed frame, a headboard for a mattress, etc. In some embodiments, the sleep enhancement device and the housing can be separate components. For example, the housing can be configured to be installed adjacent the sleep enhancement device, e.g., within a distance of at least or at most about 0.1 meters, at least or at most about 0.2 meters, at least or at most about 0.3 meters, at least or at most about 0.4 meters, at least or at most about 0.5 meters, at least or at most about 0.6 meters, at least or at most about 0.7 meters, at least or at most about 0.8 meters, at least or at most about 0.9 meters, at least or at most about 1 meter1, at least or at most about 2 meters, at least or at most about 3 meters, at least or at most about 4 meters, at least or at most about 5 meters, at least or at most about 6 meters, at least or at most about 7 meters, at least or at most about 8 meters, at least or at most about 9 meters, at least or at most about 10 meters, at least or at most about 15 meters, at least or at most about 20 meters, at least or at most about 25 meters, at least or at most about 30 meters, or at least or at most about 50 meters.

In some embodiments, the housing can be in the form of a headboard for the mattress. For example, the headboard can comprise one or more openings for the projector, the sensor, and/or the light source, as described herein, on a surface of the headboard that is configured to face a sleeping surface of the mattress.

In some embodiments, the housing can be in the form of a nightstand. This configuration can allow the sleep enhancement device to be conveniently located next to or adjacent the bed device, providing access to the at least one control option. Alternatively, the housing can be configured to be disposed on top of a nightstand.

In some embodiments, the housing can be in the form of wall furniture. This configuration can allow the housing to be incorporated into available spaces inside the room, e.g., adjacent the bed device. For example, the wall furniture can be configured to be placed at a wall that is above or to the side of at least a portion of the bed device, e.g., above the headboard of the bed device. Such wall furniture may not be a part of the bed device e.g., may not be a part of a bed frame or a headboard.

In some embodiments, the housing (e.g., comprising at least a portion of the projector and/or at least a portion of the sensor) can be installed above a bed, on a headboard, beside the bed, on a wall, on the ceiling, on a desk, on a table, on a stand, or on any surface compatible to, for example, provide the necessary detection of the user's sleep pattern and related health metrics. In some embodiments, the housing can be configured to add, remove, or modify one or more components within the housing (e.g., the projector, the sensor, or both) for various applications (e.g., different needs for different users or environments).

In some embodiments, the one or more sensors may include image sensors, motion sensors, environmental sensors, thermal sensors, audio sensors (e.g., microphones), or a combination thereof. In some embodiments, the one or more sensors may include image sensors, which can capture real-time images of the user's sleep posture, facial expressions, and movements, potentially identifying sleep disruptions or anomalies. In some embodiments, the one or more sensors may include motion sensors, which can detect subtle movements, such as restlessness or frequent tossing and turning, indicating discomfort or disturbed sleep. In some embodiments, the one or more sensors may include environmental sensors, which can monitor the sleep environment by capturing data about room temperature, humidity, and light levels. In some embodiments, the one or more sensors may include thermal sensors, which can detect changes in body temperature, aiding in the detection of potential health issues such as fever. In some embodiments, the one or more sensors may include audio sensors or microphones, which can pick up sounds such as snoring, talking during sleep, or other ambient noises that can influence the quality of sleep.

In some embodiments, a monitored space can be a personal bedroom, a shared bedroom, a hotel room, a dormitory room, or a combination thereof. In some embodiments, the system can monitor a space that includes one or more sleeping areas, sleeping zones, and/or different environments. For example, the space can be a shared bedroom and the system can be configured to monitor each individual's sleep area. As an additional example, the space can include a dormitory room, and the system can be configured to monitor each individual's bed.

In some embodiments, the monitored space can include multiple levels. For example, the monitored space may be a bunk bed, and the system can be configured to monitor each level of the bunk bed. In some embodiments, the monitored space can include indoor and/or outdoor sleeping areas. For example, the monitored space can include a camping site, and the system can be configured to capture data in each individual tent on the camping site, as well as the outdoor space included in the camping site. In some embodiments, the system can capture data under a variety of environmental conditions. For instance, the system can capture data in varying light conditions, such as during daytime, nighttime, under artificial lighting, or in a dark room. It can also operate in varying room temperatures, from cool to warm environments, and different humidity levels.

In some embodiments, one or more image sensors such as complementary metal oxide semiconductor (CMOS) or charge-coupled device (CCD) may be used for capturing image data. In some embodiments, the image sensor may be provided on a circuit board. The circuit board may be an imaging printed circuit board (PCB). The PCB may comprise one or more electronic elements for processing the image signal. For instance, the circuit for a CMOS sensor may comprise A/D converters and amplifiers to amplify and convert the analog signal provided by the CMOS sensor. In some embodiments, the image sensor may be integrated with amplifiers and converters to convert analog signal to digital signal. In some embodiments, the output of the image sensor or the circuit board may be image data (digital signals) that can be further processed by a camera circuit or processors of the camera. In some embodiments, the image sensor may comprise an array of optical sensors. In some embodiments, the camera may be a light field camera having a main lens and additional micro lens array (MLA). The light field camera model may be used to calculate a depth map of the captured image data. In some embodiments, the image data captured by the camera may be grayscale image with depth information at each pixel coordinate (i.e., depth map).

In some embodiments, the one or more sensors may comprise one or more thermal imaging sensors that can be used to perform thermal imaging. The one or more thermal imaging sensors can be passive sensors. The one or more thermal imaging sensors can detect heat signatures of humans and/or animals, fire, overheating of installations (such as accessories, wires, furniture, and/or equipment). In some embodiments, the one or more sensors can be positioned such that each individual area in the monitored space has at least one or more sensors capturing data of the individual area. In this case, each individual area of the monitored space has sleep and health data being captured from at least one or more overlapping views. As an example, a shared bedroom may be configured to include multiple sensors around each bed that provide multiple points of data collection for each sleeping individual.

In some embodiments, the system can comprise machine learning (ML) and/or artificial intelligence (AI) models used for automatically recording, analyzing, and manipulating the system. In some embodiments, ML and/or AI models can be used for sleep pattern detection. In some embodiments, the ML and/or AI models can include rule-based models, Decision Trees, KNN (k-nearest neighbor), Support Vector Machines (SVM), logistic regression models, random forest models, deep neural network (DNN) models, recurrent neural network (RNN) models, temporal pattern networks (TPN), Long Short-Term Memory Networks (LSTMs), or Fourier transform models. In some embodiments, one or more ML and/or AI models can be trained using captured data associated and/or not associated with each user. In some embodiments, training of the ML and/or AI models may comprise: collecting and developing sensors data (e.g., images, videos, temperatures) bases, feeding data to the one or more ML and/or AI models, analyzing data and identifying one or more patterns, storing and/or presenting the identified patterns, or manipulating the system (e.g., adjusting the temperature of the sleep enhancement device) by transferring commands based on the identified patterns. In some embodiments, the ML and/or AI models can be used for sleep anomaly detection, sleep stage recognition, snoring analysis, environmental analysis, sleep quality recognition, heart rate monitoring, body temperature detection, sleep disruption recognition, REM/NREM sleep recognition, movement detection, sleep position tracking, sleep disorder recognition, or a combination thereof. In some embodiments, the system can identify the user's typical sleep duration and patterns using the ML and/or AI models. For example, when for a given night, there is less sleep duration or irregular sleep patterns compared to normal sleep historical data, the system identifies sleep abnormalities and may take actions like reporting to the user, the individuals assigned by the user, health authorities, or manipulating the system (e.g., adjusting the temperature of the sleep enhancement device). In some embodiments, the system can interact and share the information (e.g., text message, email, phone call) about a given sleep disruption or health risk with other individuals rather than the user such as healthcare professionals/authorities, family members, friends, or other authorized individuals/

In some embodiments, projected data can include graphical representations of sleep stages, visualizations of body movements during sleep, charts of heart rate and body temperature variations, and alerts or notifications of detected sleep disruptions or health risks. A user-friendly display enables users to have a comprehensive understanding of their sleep patterns and health metrics. In some embodiments, the system can generate a GUI of critical sleep and health data for review of a user of the system. The GUI can include one or more tabs that can allow a user to switch between different data visualizations. For example, as displayed in, the different visualizations can include sleep stages, body movements, heart rate, body temperature, and sleep disruptions. A user may be able to interact with the tabs and the GUI can display live data of the chosen visualization. The GUI may also include a summary of the one or more sleep disruptions or health risks that have been identified. The identified disruption can be the occurrence of heavy snoring suggestive of sleep apnea. The information (e.g., live data) may provide a visual representation of the disruption to the user of the system. The projected data can include a snapshot of sleep disruption, historical sleep data showing the occurrence of the disruption, or when during the sleep session the disruption occurred. The GUI may also include a side panel that allows a user of the system to switch between different areas of the GUI to see different information, such as daily, weekly, or monthly trends, comparisons with normative data, or recommendations for sleep health improvement. The system can provide a summary of all sleep disruptions and health risks in sleep sessions associated with a user of the system in the incidents tab on a side panel. In some embodiments, a user can select from drop-down menus: (1) the sleep sessions they would like to see information for, (2) the types of statuses they would like to review (e.g., a user can select to see information for disruptions that are recurring or resolved), (3) a subset of disruptions or risks to further limit the incidents to be shown (e.g., show sleep events only associated with heavy snoring or irregular heart rate), (4) a date range, and (5) a time range.

In another aspect, the present disclosure provides computer-implemented methods performed by or implemented by any one of the systems as provided herein, to enhance sleep of the user. In some embodiments, the method can comprise adjusting the at least one sleep-related parameter for the bed device of the sleep enhancement device, thereby enhancing sleep of the user of the bed device. The bed device comprises the at least partially internal heating or cooling element. In some embodiments, the method can further comprise projecting, via the projector, the optical pattern to the wall or the ceiling adjacent the bed device. The optical pattern can provide at least one of (a) the status information for the bed device, the local environment, or both or (b) at least one control option to direct operation of the sleep enhancement device.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As utilized herein, terms “component,” “system,” “interface,” “unit” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

### Additional Details

In some embodiments, the system as provided herein can comprise at least one sensor (e.g., contact sensor) operatively coupled to the article of furniture. The at least one sensor can be attached to the article of furniture, can be part of (e.g., disposed and hidden in an internal portion of the article of furniture), or disposed near or adjacent to the article of furniture. The at least one sensor can be configured to detect sensing data associated with a user of the article of furniture. The sensing data can be associated with or indicative of a biological signal of the user. The sensing data can comprise a single biological signal data or a plurality of biological signal data. The sensing data can comprise a single type of biological signal (e.g., sound, vibration, temperature, etc.) or a plurality of different types of biological signal (e.g., sound and vibration, sound and temperature, vibration and temperature, etc.).

In some embodiments, the system can comprise a controller (e.g., a computer processor) configured to adjust the condition/operation of the article of furniture or other devices based at least in part on the sensing data (e.g., from the non-contact sensor and/or the contact sensor). The controller can be configured to generate a decision to adjust (e.g., generate a control signal for adjusting) the condition/operation of the article of furniture of other devices substantially in real-time or shortly after detection or generation of the sensing data by the at least one sensor. In some cases, the duration or time difference (e.g., a short span of time) between when such decision is made by the controller and when the sensing data is detected or generated by the at least one sensor can be at least or at most about 1 second, at least or at most about 2 seconds, at least or at most about 5 seconds, at least or at most about 10 seconds, at least or at most about 20 seconds, at least or at most about 30 seconds, at least or at most about 1 minute, at least or at most about 2 minutes, at least or at most about 5 minutes, at least or at most about 10 minutes, at least or at most about 15 minutes, at least or at most about 20 minutes, at least or at most about 25 minutes, at least or at most about 30 minutes, at least or at most about 40 minutes, at least or at most about 50 minutes, at least or at most about 60 minutes, at least or at most about 1.5 hours, or at least or at most about 2 hours prior to when the decision is generated. Alternatively, the decision to adjust the condition/operation of the article of furniture or other devices by the controller and the detection of the sensing data by the at least one sensor may not and need not occur in real-time or within a short span of time between one another as described herein.

In some embodiments, upon determining that the user may experience a target human condition (e.g., a disease such as a heart condition, or a target sleep condition such as snoring, sleep apnea, etc.), the controller can make the decision to adjust the condition/operation of the article of furniture or other devices, e.g., to treat or ameliorate such undesired condition of the user.

In some embodiments, the human condition can be a desirable condition. In some embodiments, the human condition can be an undesirable condition. In some cases, the undesirable condition can comprise a sleep disorder as provided herein.

In some embodiments, the controller can utilize at least one classifier (e.g., at least or at most about 1, 2, 3, 4, 5, 6, 7, 8, 9, 10, or more classifiers) for making the decision as provided herein, e.g., analyzing the human signal and the reference signal, analyzing the non-contact sensing data and the contact sensing data, etc. As provided herein, using the control data or making a decision based at least in part on the control data can comprise using the at least one classifier. A classifier can be configured to receive an input comprising at least the sensing data, analyze the input, and provide an output comprising, but not limited to, (i) a decision to adjust the condition/operation of the article of furniture or other devices, (ii) a target condition of the article of furniture (e.g., target temperature, target vibration state, etc.) or other devices (e.g., turning on or off), (iii) a target time to implement the adjustment of the condition/operation of the article of furniture or other devices, and/or (iv) a target duration of the implemented change of the condition/operation of the article of furniture or other devices.

In some embodiments, the controller can be configured to (e.g., via use of at least one classifier and based on the user signal as detected) determine (i) a number of users (e.g., one or two users) currently present on or adjacent to the article of furniture (e.g., on top of a bed device), (ii) an approximate or substantially precise location of each user with respect to the article of furniture (e.g., on the left side of the bed device, on the right side of the bed device, etc.), and/or (iii) identity of each user where the identity is based on information about the user previously stored in a database.

In some embodiments, an article of furniture can be associated with a user profile of a user, which user profile is stored in a database (e.g., a cloud database). The user profile can be accessible (e.g., readable, editable, etc.) by the controller as provided herein. The user profile can be accessible (e.g., readable, editable, etc.) by the user via a graphical user interface (GUI) of a user application installed on a user device. The user profile can comprise one or more adjustment profiles of the condition of the article of furniture for the user, as described herein. The user profile can comprise any additional information about the user including, but not limited to, biological sex, age, height, weight, medical history, family history, identity of family members, genetic information, blood information, information about additional article(s) of furniture of the same user or other users (e.g., other members of the cohort as provided herein), etc. The additional information can be provided by the user. The additional information can be retrieved from another database associated with the user (e.g., a database associated with the user's smart watch, with the user's genetic analysis such as 23andMe, etc.).

In some embodiments, the controller can be configured to compute a score indicative of a quality of the user's usage of the article of furniture, based at least in part on the sensing data (e.g., the non-contact sensing data). For example, after usage, the user can provide feedback or input via GUI associated with the controller about how the user perceives the usage was like, and the controller can utilize such information to compute the score. In some embodiments, the article of furniture can be a bed device, and the controller can be configured to compute a sleep score indicative of a sleep quality of the user while sleeping on the bed device.

In some embodiments, the sleep score as computed by the controller can be relative to a threshold (or benchmark) sleep score. The threshold sleep score can be based on a compiled sleep score and/or sensing data from a population of users (e.g., at least about 10, 50, 100, 500, 1,000, 5,000, 10,000, 20,000, 50,000, or more users) collected over the course of days, weeks, months, or years (e.g., at least or at most about 1 year, at least or at most about 2 years, at least or at most about 3 years, at least or at most about 4 years, at least or at most about 5 years, etc.). Alternatively, in some embodiments, the sleep score as computed by the controller can be more individualized, and the threshold sleep score can be based on a compiled sleep score and/or sensing data from the specific user collected, for example, collected longitudinally, over the course of days, weeks, months, or years (e.g., at least or at most about 1 year, at least or at most about 2 years, at least or at most about 3 years, at least or at most about 4 years, at least or at most about 5 years, etc.).

In some embodiments, a score (e.g., sleep score) as provided herein can be a rating. The rating can be a numerical rating, alphabetical rating, alphanumerical rating, percentage, graphical system, etc. In some cases, the ratings can be provided based on a scale, wherein a higher rating can be associated with a better user experience (e.g., a better sleep quality) as compared to a lower rating. In some cases, the rating can be based on a 0 percent (%) to 100 % scale, a higher percentage value representing a better user experience (e.g., a better sleep quality). The rating can be at least about 0%, 1%, 2%, 3%, 4%, 5%, 6%, 7%, 8%, 9%, 10%, 20%, 30%, 40%, 50%, 60%, 70%, 80%, 90%, 99%, or more. The rating can be at most about 100%, 90%, 80%, 70%, 60%, 50%, 40%, 30%, 20%, 10%, 9%, 8%, %, 6%, 5%, 4%, 3%, 2%, 1%, or less. In some cases, the rating can be based on another numerical scale, such as, for example, a 0 to 10 scale (e.g., 0, 1, 2, 3, 4, 5, 6, 7, 8, 9, and 10 in an order of positive effects). In some cases, the rating can be based on a graphical scale, such as, for example, a 1 to 5 stars scale (e.g., 1, 2, 3, 4, and 5 starts in an order of positive effects). In some cases, the rating can be an alphabetical rating system, such as, for example, one or more of D−, D, D+, C−, C, C+, B−, B, B+, A−, A, and A+.

In some embodiments, the systems and methods of the present disclosure can be utilized (e.g., via the article of furniture and/or the controller) to control other devices (or instruments as used interchangeably herein) in the environment or associated with the user. Non-limiting examples of the other devices can include, but are not limited to, an alarm, a coffee machine, a lock, a user device (e.g., a mobile phone, a personal computer, a smart watch, etc.), a car, an exercise machine, etc.

Another aspect of the present disclosure provides a system comprising one or more computer processors and computer memory coupled thereto. The computer memory comprises machine executable code that, upon execution by the one or more computer processors, implements any of the methods above or elsewhere herein such as, for example, one or more operations of the sensor unit as provided herein.

### Longitudinal Health Monitoring System and Methods

In some aspects, the present disclosure provides systems and methods for detecting a condition of a user. The user may use an article of furniture (e.g., a bed device) described herein. For example, a user may use a mattress and/or mattress cover described herein. The systems and methods may measure one or more physiological data signatures (e.g., biomarkers) of the use. The one or more physiological data signatures (e.g., biomarkers) may comprise any data signatures (e.g., biomarkers) related to breathing and heart function. The systems and methods may measure a respiratory rate, a heart rate, a heart rate variability (HRV), or any combination thereof. The one or more physiological data signatures (e.g., biomarkers) may be derived from one or more signals measured by a sensors or multiple sensors of the article of furniture. Non-limiting examples of a sensor can include a capacitance sensor, a temperature sensor, a pressure sensor, a piezoelectric sensor, sound sensor (e.g., a microphone), accelerometer, liquid pressure sensor, or any combination thereof. The sensor may be disposed on the article of furniture (e.g., on a top, a side, or a bottom of the article of furniture). In some embodiments, the sensor may be not be disposed on the article of furniture. There are multiple advantages for using a piezoelectric sensor. For example, the piezoelectric sensor does not need to contact the user, which may reduce a user burden. A piezoelectric sensor can have high sensitivity which can detect even minor variations in respiratory rate. A piezoelectric sensor can be used for long-term monitoring which may enable continuous sleep tracking over multiple nights.

The systems and methods described herein can detect breathing of a user and/or a breathing anomaly (e.g., an apnea event). In some embodiments, detection of a condition (e.g., a breathing disturbance, cardiac anomaly, or any combination thereof) of a user may comprise using a single stage computer algorithm. In some embodiments, detection of a condition (e.g., a breathing disturbance, cardiac anomaly, or any combination thereof) of a user may comprise using a multi-stage computer algorithm. For example, an algorithm may be at least about one stage, two-stages, three-stages, four-stages, five-stages, or greater than about five-stages. A multi-stage algorithm may be advantageous as it may enhance accuracy of the detection as compared to a detection algorithm using a single stage (e.g., one algorithm).

In some embodiments, a computer algorithm (e.g., a first computer algorithm) can be used to identify one or more physiological data signatures (e.g., biomarkers) of the user. The computer algorithm (e.g., the first computer algorithm) may preprocess a biosignal generated by a sensor. For example, the computer algorithm (e.g., a first computer algorithm) may preprocess a raw piezoelectric signal. Preprocessing may comprise downsampling, filtering, noise reduction, or any combination thereof. A bandpass filter can be applied to further isolate ranges of known bioactivity (e.g., respiration and/or cardiac activity). For example, a bandpass filter can be applied at a range from about 0.1 Hz to about 2 Hz for respiratory detection. In some embodiments, a bandpass filter can be applied at a range from about 0.1 Hz to about 0.2 Hz, about 0.1 Hz to about 0.3 Hz, about 0.1 Hz to about 0.4 Hz, about 0.1 Hz to about 0.5 Hz, about 0.1 Hz to about 0.6 Hz, about 0.1 Hz to about 0.7 Hz, about 0.1 Hz to about 0.8 Hz, about 0.1 Hz to about 0.9 Hz, about 0.1 Hz to about 1 Hz, about 0.1 Hz to about 1.5 Hz, about 0.1 Hz to about 2 Hz, about 0.2 Hz to about 0.3 Hz, about 0.2 Hz to about 0.4 Hz, about 0.2 Hz to about 0.5 Hz, about 0.2 Hz to about 0.6 Hz, about 0.2 Hz to about 0.7 Hz, about 0.2 Hz to about 0.8 Hz, about 0.2 Hz to about 0.9 Hz, about 0.2 Hz to about 1 Hz, about 0.2 Hz to about 1.5 Hz, about 0.2 Hz to about 2 Hz, about 0.3 Hz to about 0.4 Hz, about 0.3 Hz to about 0.5 Hz, about 0.3 Hz to about 0.6 Hz, about 0.3 Hz to about 0.7 Hz, about 0.3 Hz to about 0.8 Hz, about 0.3 Hz to about 0.9 Hz, about 0.3 Hz to about 1 Hz, about 0.3 Hz to about 1.5 Hz, about 0.3 Hz to about 2 Hz, about 0.4 Hz to about 0.5 Hz, about 0.4 Hz to about 0.6 Hz, about 0.4 Hz to about 0.7 Hz, about 0.4 Hz to about 0.8 Hz, about 0.4 Hz to about 0.9 Hz, about 0.4 Hz to about 1 Hz, about 0.4 Hz to about 1.5 Hz, about 0.4 Hz to about 2 Hz, about 0.5 Hz to about 0.6 Hz, about 0.5 Hz to about 0.7 Hz, about 0.5 Hz to about 0.8 Hz, about 0.5 Hz to about 0.9 Hz, about 0.5 Hz to about 1 Hz, about 0.5 Hz to about 1.5 Hz, about 0.5 Hz to about 2 Hz, about 0.6 Hz to about 0.7 Hz, about 0.6 Hz to about 0.8 Hz, about 0.6 Hz to about 0.9 Hz, about 0.6 Hz to about 1 Hz, about 0.6 Hz to about 1.5 Hz, about 0.6 Hz to about 2 Hz, about 0.7 Hz to about 0.8 Hz, about 0.7 Hz to about 0.9 Hz, about 0.7 Hz to about 1 Hz, about 0.7 Hz to about 1.5 Hz, about 0.7 Hz to about 2 Hz, about 0.8 Hz to about 0.9 Hz, about 0.8 Hz to about 1 Hz, about 0.8 Hz to about 1.5 Hz, about 0.8 Hz to about 2 Hz, about 0.9 Hz to about 1 Hz, about 0.9 Hz to about 1.5 Hz, about 0.9 Hz to about 2 Hz, about 1 Hz to about 1.5 Hz, about 1 Hz to about 2 Hz, or about 1.5 Hz to about 2 Hz for respiratory detection.

In other embodiments, a bandpass filter can be applied at a range from about 3 Hz to about 25 Hz for detection of cardiac activity. A bandpass filter can be applied at a range from about 3 Hz to about 4 Hz, about 3 Hz to about 5 Hz, about 3 Hz to about 6 Hz, about 3 Hz to about 7 Hz, about 3 Hz to about 8 Hz, about 3 Hz to about 9 Hz, about 3 Hz to about 10 Hz, about 3 Hz to about 15 Hz, about 3 Hz to about 18 Hz, about 3 Hz to about 20 Hz, about 3 Hz to about 25 Hz, about 4 Hz to about 5 Hz, about 4 Hz to about 6 Hz, about 4 Hz to about 7 Hz, about 4 Hz to about 8 Hz, about 4 Hz to about 9 Hz, about 4 Hz to about 10 Hz, about 4 Hz to about 15 Hz, about 4 Hz to about 18 Hz, about 4 Hz to about 20 Hz, about 4 Hz to about 25 Hz, about 5 Hz to about 6 Hz, about 5 Hz to about 7 Hz, about 5 Hz to about 8 Hz, about 5 Hz to about 9 Hz, about 5 Hz to about 10 Hz, about 5 Hz to about 15 Hz, about 5 Hz to about 18 Hz, about 5 Hz to about 20 Hz, about 5 Hz to about 25 Hz, about 6 Hz to about 7 Hz, about 6 Hz to about 8 Hz, about 6 Hz to about 9 Hz, about 6 Hz to about 10 Hz, about 6 Hz to about 15 Hz, about 6 Hz to about 18 Hz, about 6 Hz to about 20 Hz, about 6 Hz to about 25 Hz, about 7 Hz to about 8 Hz, about 7 Hz to about 9 Hz, about 7 Hz to about 10 Hz, about 7 Hz to about 15 Hz, about 7 Hz to about 18 Hz, about 7 Hz to about 20 Hz, about 7 Hz to about 25 Hz, about 8 Hz to about 9 Hz, about 8 Hz to about 10 Hz, about 8 Hz to about 15 Hz, about 8 Hz to about 18 Hz, about 8 Hz to about 20 Hz, about 8 Hz to about 25 Hz, about 9 Hz to about 10 Hz, about 9 Hz to about 15 Hz, about 9 Hz to about 18 Hz, about 9 Hz to about 20 Hz, about 9 Hz to about 25 Hz, about 10 Hz to about 15 Hz, about 10 Hz to about 18 Hz, about 10 Hz to about 20 Hz, about 10 Hz to about 25 Hz, about 15 Hz to about 18 Hz, about 15 Hz to about 20 Hz, about 15 Hz to about 25 Hz, about 18 Hz to about 20 Hz, about 18 Hz to about 25 Hz, or about 20 Hz to about 25 Hz for detection of cardiac activity.

An algorithm can be a breath detection algorithm. In some embodiments, an algorithm (e.g., a breath detection algorithm) may analyze the sensing data from the user (e.g., the respiratory data) and determine breath cycles. In some embodiments, the algorithm may identify breath cycles using peak detection. In some embodiments, the algorithm may perform inter-breath interval (IBI) computation to estimate a respiration rate of the user. The respiration rate may be based on consecutive troughs in the sensing data. The algorithm may perform a breath variability assessment. The breath variability assessment may detect irregular patterns in the sensing data. The irregular patterns may be an indication of disordered breathing (e.g., an apnea event).

An algorithm can be a heartbeat detection algorithm. In some embodiments, an algorithm (e.g., a heartbeat detection algorithm) may analyze one or more characteristics of a user's heartbeat. In some embodiments, an algorithm (e.g., a heartbeat detection algorithm) may isolate a cardiac component from a piezoelectric signal (e.g., a raw piezoelectric signal). The algorithm may comprise a model (e.g., a trained deep learning model) to detect heartbeat, compute heart variability (HRV), or any combination thereof. The algorithm (e.g., the heartbeat detection algorithm) may use an amplitude of a heartbeat to distinguish a condition (e.g., an apnea event) from movement or sensor displacement. For example, the algorithm may use low-amplitude heartbeats to distinguish apnea from sensor displacement.

Processing of a signal detected from a sensor herein may comprise identification of breathing cessation. For example, breathing cessation may be identified when a breath gap exceeds at least about 1 second, at least about 2 seconds, at least about 3 seconds, at least about 3 seconds, at least about 4 seconds, at least about 5 seconds, at least about 6 seconds, at least about 7 seconds, at least about 8 seconds, at least about 9 seconds, at least about 10 seconds, at least about 12 seconds, at least about 15 seconds, at least about 20 seconds, or greater than about 20 seconds. Breathing cessation may be identified when a breath gap exceeds at most about 20 seconds, at most about 15 seconds, at most about 12 seconds, at most about 10 seconds, at most about 9 seconds, at most about 8 seconds, at most about 7 seconds, at most about 6 seconds, at most about 5 seconds, at most about 4 seconds, at most about 3 seconds, at most about 2 seconds, at most about 1 second, or less than about 1 second. In some embodiments, breathing cessation may be identified when a breath gap is from about 1 second to about 15 seconds. In some embodiments, breathing cessation may be identified when a breath gap is from about 1 second to about 2 seconds, about 1 second to about 3 seconds, about 1 second to about 4 seconds, about 1 second to about 5 seconds, about 1 second to about 6 seconds, about 1 second to about 7 seconds, about 1 second to about 8 seconds, about 1 second to about 9 seconds, about 1 second to about 10 seconds, about 1 second to about 12 seconds, about 1 second to about 15 seconds, about 2 seconds to about 3 seconds, about 2 seconds to about 4 seconds, about 2 seconds to about 5 seconds, about 2 seconds to about 6 seconds, about 2 seconds to about 7 seconds, about 2 seconds to about 8 seconds, about 2 seconds to about 9 seconds, about 2 seconds to about 10 seconds, about 2 seconds to about 12 seconds, about 2 seconds to about 15 seconds, about 3 seconds to about 4 seconds, about 3 seconds to about 5 seconds, about 3 seconds to about 6 seconds, about 3 seconds to about 7 seconds, about 3 seconds to about 8 seconds, about 3 seconds to about 9 seconds, about 3 seconds to about 10 seconds, about 3 seconds to about 12 seconds, about 3 seconds to about 15 seconds, about 4 seconds to about 5 seconds, about 4 seconds to about 6 seconds, about 4 seconds to about 7 seconds, about 4 seconds to about 8 seconds, about 4 seconds to about 9 seconds, about 4 seconds to about 10 seconds, about 4 seconds to about 12 seconds, about 4 seconds to about 15 seconds, about 5 seconds to about 6 seconds, about 5 seconds to about 7 seconds, about 5 seconds to about 8 seconds, about 5 seconds to about 9 seconds, about 5 seconds to about 10 seconds, about 5 seconds to about 12 seconds, about 5 seconds to about 15 seconds, about 6 seconds to about 7 seconds, about 6 seconds to about 8 seconds, about 6 seconds to about 9 seconds, about 6 seconds to about 10 seconds, about 6 seconds to about 12 seconds, about 6 seconds to about 15 seconds, about 7 seconds to about 8 seconds, about 7 seconds to about 9 seconds, about 7 seconds to about 10 seconds, about 7 seconds to about 12 seconds, about 7 seconds to about 15 seconds, about 8 seconds to about 9 seconds, about 8 seconds to about 10 seconds, about 8 seconds to about 12 seconds, about 8 seconds to about 15 seconds, about 9 seconds to about 10 seconds, about 9 seconds to about 12 seconds, about 9 seconds to about 15 seconds, about 10 seconds to about 12 seconds, about 10 seconds to about 15 seconds, or about 12 seconds to about 15 seconds.

A length of a breath gap can be cross-referenced with other data to better classify a user condition (e.g., an apnea event). For example, one or more breath gaps can be cross-referenced with heartbeats (e.g., low-amplitude heartbeats) to determine a breathing disturbance (e.g., an apnea event). A breathing disturbance (e.g., an apnea event) can be determined when a breathing anomaly and/or a cardiac anomaly persist in a user. A breathing disturbance (e.g., an apnea event) can be determined when a breathing anomaly and/or a cardiac anomaly persist in a user beyond a threshold. A threshold can be a duration of time that an event lasts and/or a number of occurrences of an event over a period of time.

In some embodiments, the systems and methods described herein can comprise a two-stage algorithm. A first stage (e.g., first algorithm) may comprise an algorithm described herein used to process a biosignal of a user. For example, a first algorithm may identify one or more breath gaps of a user, wherein the breath gaps are from data generated by a piezoelectric sensor. A second stage algorithm (e.g., a second algorithm) may use a machine learning model (e.g., a deep learning residual neural network model (ResNet model)) to verify the classifications of the first algorithm. The machine learning model can be trained on training data comprising annotated samples. The model may be performed in real-time, wherein it may be performed almost immediately or within a short enough time span, such as within at least 0.0001 milliseconds, 0.0005 milliseconds, 0.001 milliseconds, 0.005 milliseconds, 0.01 milliseconds, 0.05 milliseconds, 0.1 milliseconds, 0.5 milliseconds, 1 millisecond, 5 milliseconds, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 milliseconds, 1 millisecond, 0.5 milliseconds, 0.1 milliseconds, 0.05 milliseconds, 0.01 milliseconds, 0.005 milliseconds, 0.001 milliseconds, 0.0005 milliseconds, 0.0001 milliseconds, or less.

The combination of the second algorithm comprising the machine learning model (e.g., ResNet model) with the first algorithm may enhance accuracy of detection. For example, a first algorithm may comprise an at least about 30% positive predictive value, at least about 40% positive predictive value, at least about 50% positive predictive value, at least about 60% positive predictive value, at least about 65% positive predictive value, at least about 70% positive predictive value, at least about 75% positive predictive value, at least about 80% positive predictive value, at least about 85% positive predictive value, at least about 90% positive predictive value, or greater than about 90% positive predictive value in detecting a condition of a user (e.g., a breathing disturbance and/or cardiac anomaly).

A receiver operating characteristic (ROC) curve is a graph that shows how well a model can distinguish between positive and negative outcomes. A higher area under a ROC curve can identify a model with higher accuracy. In some embodiments, a two-stage algorithm described herein (e.g., with a second algorithm comprising the machine learning model) can achieve an area under the ROC curve of at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or greater than about 0.99. In some embodiments, a two-stage algorithm described herein (e.g., with a second algorithm comprising the machine learning model) can achieve an area under the ROC curve of at most about 0.99, at most about 0.98, at most about 0.97, at most about 0.96, at most about 0.95, at most about 0.94, at most about 0.93, at most about 0.92, at most about 0.91, at most about 0.90, at most about 0.85, at most about 0.80, at most about 0.75, at most about 0.70, or less than about 0.70. In some embodiments, a two-stage algorithm described herein (e.g., with a second algorithm comprising the machine learning model) can achieve an area under the ROC curve from about 0.5 to about 0.99. In some embodiments, a two-stage algorithm described herein (e.g., with a second algorithm comprising the machine learning model) can achieve an area under the ROC curve from about 0.5 to about 0.6, about 0.5 to about 0.7, about 0.5 to about 0.8, about 0.5 to about 0.85, about 0.5 to about 0.9, about 0.5 to about 0.92, about 0.5 to about 0.94, about 0.5 to about 0.96, about 0.5 to about 0.97, about 0.5 to about 0.98, about 0.5 to about 0.99, about 0.6 to about 0.7, about 0.6 to about 0.8, about 0.6 to about 0.85, about 0.6 to about 0.9, about 0.6 to about 0.92, about 0.6 to about 0.94, about 0.6 to about 0.96, about 0.6 to about 0.97, about 0.6 to about 0.98, about 0.6 to about 0.99, about 0.7 to about 0.8, about 0.7 to about 0.85, about 0.7 to about 0.9, about 0.7 to about 0.92, about 0.7 to about 0.94, about 0.7 to about 0.96, about 0.7 to about 0.97, about 0.7 to about 0.98, about 0.7 to about 0.99, about 0.8 to about 0.85, about 0.8 to about 0.9, about 0.8 to about 0.92, about 0.8 to about 0.94, about 0.8 to about 0.96, about 0.8 to about 0.97, about 0.8 to about 0.98, about 0.8 to about 0.99, about 0.85 to about 0.9, about 0.85 to about 0.92, about 0.85 to about 0.94, about 0.85 to about 0.96, about 0.85 to about 0.97, about 0.85 to about 0.98, about 0.85 to about 0.99, about 0.9 to about 0.92, about 0.9 to about 0.94, about 0.9 to about 0.96, about 0.9 to about 0.97, about 0.9 to about 0.98, about 0.9 to about 0.99, about 0.92 to about 0.94, about 0.92 to about 0.96, about 0.92 to about 0.97, about 0.92 to about 0.98, about 0.92 to about 0.99, about 0.94 to about 0.96, about 0.94 to about 0.97, about 0.94 to about 0.98, about 0.94 to about 0.99, about 0.96 to about 0.97, about 0.96 to about 0.98, about 0.96 to about 0.99, about 0.97 to about 0.98, about 0.97 to about 0.99, or about 0.98 to about 0.99.

In some embodiments, the systems and methods described herein can be used for long-term monitoring of sleep health (e.g., monitoring of respiratory health and/or cardiac health). The systems and methods described herein can be used to measure one or more data signatures (e.g., biomarkers) of a user over a time period of at least about 30 seconds, at least about 1 minute, at least about 2 minutes, at least about 3 minutes, at least about 4 minutes, at least about 5 minutes, at least about 10 minutes, at least about 30 minutes, at least about 1 hour, at least about 2 hours, at least about 3 hours, at least about 4 hours, at least about 5 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 3 months, at least about 6 months, at least about 1 year, at least about 2 years, at least about 3 years, at least about 4 years, at least about 5 years, or greater than about 5 years. The systems and methods described herein can be used to measure one or more data signatures (e.g., biomarkers) of a user over a time period of at most about 5 years, at most about 4 years, at most about 3 years, at most about 2 years, at most about 1 year, at most about 6 months, at most about 3 months, at most about 1 month, at most about 4 weeks, at most about 3 weeks, at most about 2 weeks, at most about 1 week, at most about 6 days, at most about 5 days, at most about 4 days, at most about 3 days, at most about 2 days, at most about 1 day, at most about 12 hours, at most about 6 hours, at most about 5 hours, at most about 4 hours, at most about 3 hours, at most about 2 hours, at most about 1 hour, at most about 30 minutes, at most about 10 minutes, at most about 5 minutes, at most about 4 minutes, at most about 3 minutes, at most about 2 minutes, at most about 1 minutes, at most about 30 seconds, or less than about 30 seconds.

For example, provided herein are computer-implemented method for detecting a condition of a user of an article of furniture, the method comprising: determining, by a first computer algorithm, one or more sensor data signatures indicative of a physiological condition of the user via based on an input sensor data associated with a biological signal of the user, wherein the input sensor data is collected by a biological sensor while the user is using (e.g., sleeping on) the article of furniture; and confirming, by a second and different computer algorithm, the determined one or more sensor data signatures based on (i) at least a portion of the input sensor data and (ii) the determined one or more sensor data signatures.

The first computer algorithm can be configured to extract one or more data signatures from raw data. The first computer algorithm can be digital signal processing (DSP). The first computer algorithm can be configured to extract respiratory data and/or cardiac data from the user based on one or more signals generated by sensors of the article of furniture. For example, the first computer algorithm can extract respiratory rate from piezoelectric signal generated by a piezoelectric sensor of the article of furniture (e.g., bed device). As another example, the first computer algorithm can extract heart rate from piezoelectric signal generated by a piezoelectric sensor of the article of furniture (e.g., bed device).

In some embodiments, the second computer algorithm may be a residual neural network (ResNet) model, a convolution neural network, an Exponential Moving Average (EMA) model, or any combination thereof. In some embodiments, the second computer algorithm may comprise one or more models (e.g., 1, 2, 3, 4, 5, 6, 7, 8, 9, 10, or more models). A model and/or machine learning algorithm may be trained on a data set. The data set can comprise a number of breathing disturbance detections (e.g., apnea detections) and/or cardiac detections (e.g., arrhythmia detections) from one or more sensors (e.g., one or more piezo sensors). In some embodiments, the second computer algorithm can be trained using supervised labeling (e.g., human labeling).

In some embodiments, the input sensor data can comprise respiratory data and/or cardiac data. In some embodiments, the second computer algorithm can be different from the first computer algorithm. In some embodiments, the second computer algorithm can be the same as the first computer algorithm. The method may further comprise providing (i) the at least the portion of the input sensor data and (ii) the determined one or more sensor data signatures as inputs for the second and different computer algorithm. In some embodiments, the at least the portion of the input sensor data and the determined one or more sensor data signatures can be provided simultaneously as inputs to the second and different computer algorithm.

The second computer algorithm may be configured to confirm the one or more data signatures determined by the first computer algorithm. The second computer algorithm may calculate an accuracy of the one or more data signatures determined by the first computer algorithm. In some embodiments, the methods can further comprise determining a physiological condition of the user. The physiological condition may be determined by the second computer algorithm. One or more data signatures of the user may comprise respiratory rate, breath cycle, heart rate, heart rate variability (HRV), vibration, perspiration, blood pressure, body temperature, or any combination thereof. A vibration may be a snoring vibration. In some embodiments, the one or more data signatures determined by a computer algorithm described herein (e.g., a first computer algorithm) can be based on biological signals of the user. The biological signals can be derived from any vital sign of the user (e.g., respiration, cardiac activity, body temperature, or combinations thereof). The data signatures may be produced at least once per minute while a user is using (e.g., sleeping on) the article of furniture. The data signatures may be produced at most once per minute while a user is using (e.g., sleeping on) the article of furniture. In some embodiments, at least about 1, 2, 3, 4, 5, 6, 7, 8, 9, 10, 20, 30, 40, 50, 60, or greater than about 60 data signatures may be produced per minute by a user. In some embodiments, at least about 1, 2, 3, 4, 5, 6, 7, 8, 9, 10, 20, 30, 40, 50, 60, or greater than about 60 data signatures may be produced per hour by a user. In some embodiments, at most about 60, 50, 40, 30, 20, 10, 9, 8, 7, 6, 5, 4, 3, 2, 1, or less than about 1 data signatures may be produced per minute by a user. In some embodiments, at most about 60, 50, 40, 30, 20, 10, 9, 8, 7, 6, 5, 4, 3, 2, 1, or less than about 1 data signatures may be produced per hour by a user. In some embodiments, the input sensor data may be collected continuously while a user is using (e.g., sleeping on) the article of furniture. In some embodiments, the input sensor data may not be collected continuously while a user is using (e.g., sleeping on) the article of furniture.

A biological sensor can be any biological sensor (e.g., sensing device) described herein. For example, a biological sensor used to collect the input sensor data can be a piezoelectric sensor, a temperature sensor, a ballistocardiograph, or any combination thereof. In some embodiments, the biological sensor can comprise a piezoelectric sensor. In some embodiments, the biological sensor can comprise one or more sensors (e.g., 1, 2, 3, 4, 5, 6, 7, 8, 9, 10, or more sensors). The one or more sensors may all be the same type of sensor (e.g., the sensors may be all piezoelectric sensors). In some embodiments, the one or more sensors may be different sensors (e.g., the sensors may be a combination of piezoelectric sensors, temperature sensors, and/or ballistocardiographs).

In some embodiments, the first computer algorithm may comprise downsampling the input sensor data. This may be advantageous to reduce computational complexity while preserving essential signal features. For example, a raw signal (e.g., raw piezoelectric signal) may be collected at 500 Hz and may be downsampled to 100 Hz. The first computer algorithm can further provide a bandpass filter as described herein to isolate respiratory and/or cardiac activity in the input sensor data. The first computer algorithm can comprise a breath detection algorithm and/or a heartbeat detection algorithm as described herein.

In some embodiments, the condition of the user can be a cardio-respiratory condition. The condition of the user can be apnea, arrhythmia, flu, cold, fever, insomnia, or any combination thereof. The condition of the user can be atrial fibrillation, atrial flutter, ventricular tachycardia, ventricular fibrillation, or combinations thereof. In some embodiments, the condition of the user may be a physiological stressor. The physiological stressor may be poor sleep, insomnia, alcohol intake, exercise, smoking, or any combination thereof. The computer algorithm described herein can be used to identify a wellbeing of a user and one or more abnormalities in the cardio-respiratory health of a user.

The first computer algorithm can be configured to extract one or more data signatures from raw data signal (e.g., biological signal collected from a sensor of an article of furniture). A second computer algorithm can comprise a model (e.g., an exponential moving average (EMA) model) assigned to each data signature. For example, if a first computer algorithm extracts a heart rate and a respiratory rate from raw data signal, a second computer algorithm may assign an EMA model to the heart rate and an EMA model to the respiratory rate. The second computer algorithm may comprise an EMA model for each data signature. The second computer algorithm may comprise an EMA model for each data signature across each day of the week. For example, the second computer algorithm may comprise an EMA model for a heart rate metric and an EMA model for a respiratory rate metric, and then each of those models for each day of the week (e.g., 14 total EMA models). These distinct models for each day of the week can be advantageous as it can allow the models to capture the weekly behavioral trend that occurs due to weekday and weekend and separate the variable behavior from the underlying physiological health of the user.

A second computer algorithm (e.g., an EMA model) can comprise a mean, variance, history, or any combination thereof. In some embodiments, a second computer algorithm (e.g., an EMA model) can comprise a mean, variance, and history. A mean value may be an estimated value for the data signature for any period of time (e.g., a day, a week, etc.). A variance (e.g., standard deviation) value may be an estimated value for the data signature for any period of time (e.g., a day, a week, etc.). A history value can be a number of days used to compute the mean and./or variance.

A computer algorithm may compare a data signature extracted from an input sensor data with a historical value of a user. For example, a computer algorithm may compare whether a respiratory rate of a user during a sleep session differs from a historical respiratory rate of the user. The computer algorithm may generate a notification if a value for the data signature (e.g., respiratory rate) differs strongly from a historical value (e.g., expected value) for that data signature. For example, the computer algorithm may generate a notification if a value for the data signature (e.g., respiratory rate) differs by more than 1 standard deviation, 2 standard deviations, 3 standard deviations, 4 standard deviations, or 5 standard deviations away from the historical value (e.g., expected value) for that data signature.

As another example, provided herein are computer-implemented method for evaluating a condition of a user of an article of furniture, the method comprising: predicting a future sensor data or analysis thereof (future data) based on a historical sensor data set, wherein the future data is indicative of a physiological condition of the user; and determining a difference between the predicted future data and a new data, wherein the difference is indicative of an anomaly of the physiological condition of the user.

In some embodiments, the methods may evaluate one or more conditions of a user. The condition may be a cardiac condition, a respiratory condition, or any combination thereof. The condition may a physiological stressor described herein. In some embodiments, the systems and/or methods described herein may evaluate 1, 2, 3, 4, 5, 6, 7, 8, 9, 10, or more conditions of a user. In some embodiments, the systems and/or methods described herein may evaluate one or more conditions of multiple users (e.g. two or more users) of an article of furniture. The method may comprise a computer algorithm. The computer algorithm may predict a future data. The future data may be based on a historical sensor data set. The historical sensor data set may be from the user of the article of furniture (e.g., bed device). In some embodiments, the historical sensor data set may not be from the user of the article of furniture (e.g., bed device). The historical sensor data set may be collected from multiple users (e.g., 1, 2, 3, 4, 5, 6, 7, 8, 9, 10, 15, 20, 30, 40, 50, 100, or more than 100 users).

In some embodiments, the historical sensor data set may be collected from one or more previous sleep sessions (e.g., previous sleep sessions of a user of the article of furniture). The future data may comprise future data signatures. The future data and/or future data signatures can comprise a respiratory rate of a user, a breath cycle of a user, a heart rate of a user, a heart rate variability of a user, a vibration, or any combination thereof. In some embodiments, the future data comprises at least about 1 future data signature, at least about 2 future data signatures, at least about 3 future data signatures, at least about 4 future data signatures, at least about 5 future data signatures, at least about 10 future data signatures, at least about 15 future data signatures, at least about 20 future data signatures, or greater than about 20 future data signatures. In some embodiments, the future data comprises at most about 20 future data signatures, at most about 15 future data signatures, at most about 10 future data signatures, at most about 5 future data signatures, at most about 4 future data signatures, at most about 3 future data signatures, at most about 2 future data signatures, at most about 1 future data signature, or less than about 1 future data signature.

The future data can be indicative of a physiological condition of a user. In some embodiments, the future data can be indicative of one or more physiological conditions of a user. The physiological condition may be a respiratory condition, a cardiac condition, a sleep condition, or any combination thereof. The methods provided herein may comprise determining a difference between a predicted future data (e.g., an expected value) and a new data (e.g., a current value from a current sleep session). The predicted future data and the new data may be the same type of data. The predicted future data and the new data may not be the same type of data. For example, the predicted future data may be a respiratory rate and the new data may be a respiratory rate. As another example, the predicted future data may be a respiratory rate and a heart rate, and the the new data may be a respiratory rate and a heart rate.

A difference in the predicted future data and the new data may be indicative of an anomaly of the physiological condition of the user. The anomaly may be an anomaly of a health condition. The difference in the predicted future data and the new data may be indicative of an anomaly in respiratory health, cardiac health, or any combination thereof. For example, a difference in the predicted future data and the new data may be indicative of an apnea event. As another example, a difference in the predicted future data and the new data may be indicative of an arrhythmia.

The methods described herein may comprise generating a report. The report can comprise a representation of the anomaly of the physiological condition of the user. The report may be presented via a user interface (e.g., a graphical user interface) described herein. The report may inform a user of how many anomalies are detected (e.g., how many anomalies are detected over a sleep session). In some embodiments, a report may be generated if there is an anomaly. In some embodiments, a report may be generated if there is no anomaly. In some embodiments, a report may not be generated if there is no anomaly.

A report may generated if a difference between the predicted future data and a new data is of an order of magnitude. For example, if there is a large difference between the predicted future data and a new data, a report may be generated. If there is a small difference between the predicted future data and a new data, a report may not be generated. If there is a small difference between the predicted future data and a new data, a report may be generated. A report may be generated when the difference between a predicted future data (e.g., an expected value for a user's respiratory rate, heart rate, HRV, or combination thereof) and a new data (e.g., a current value for a user's respiratory rate, heart rate, HRV, or combination thereof) deviates by at least about 1 standard deviation, at least about 2 standard deviations, at least about 3 standard deviations, or greater than about 3 standard deviations. A report may be generated when the difference between a predicted future data (e.g., an expected value for a user's respiratory rate, heart rate, HRV, or combination thereof) and a new data (e.g., a current value for a user's respiratory rate, heart rate, HRV, or combination thereof) deviates by greater than about 1 standard deviation. A report may be generated when the difference between a predicted future data (e.g., an expected value for a user's respiratory rate, heart rate, HRV, or combination thereof) and a new data (e.g., a current value for a user's respiratory rate, heart rate, HRV, or combination thereof) deviates by greater than about 2 standard deviations.

In some embodiments, the predicting may be performed by a computer algorithm. The computer algorithm may be one or more Exponential Moving Average (EMA) models. In some embodiments, the computer algorithm may be at least about 1, 2, 3, 4, 5, 10, 15, 20, 25, or greater than about 25 EMA models. The methods described herein may further comprise determining the new data (e.g., determining a current value of a user's respiratory rate, heart rate, HRV, or combination thereof during a sleep session). Determining a user's respiratory rate, heart rate, HRV, or combination thereof during a sleep session may be performed by a computer algorithm. In some embodiments, the computer algorithm determining a user's new data may be the same computer algorithm predicting the future data. In some embodiments, the computer algorithm determining a user's new data may be different from the computer algorithm predicting the future data. Determining the new data may comprise extracting data signatures from a raw data. The raw data may be input sensor data associated with a biological signal of a user.

In some embodiments, the input sensor data can be collected by one or more biological sensors. The one or more biological sensors may be coupled to the article of furniture (e.g., the bed device). The input sensor data may be collected while a user is using the article of furniture (e.g., the bed device). The input sensor data may be collected while a user is sleeping on the article of furniture (e.g., the bed device). In some embodiments, the biological sensor may comprise a piezoelectric sensor, a temperature sensor, a ballistocardiograph, or any combination thereof.

In some embodiments, one or more median values may be calculated from the new data and/or new data signatures. For example, if five respiratory rates are collected over a user's sleep session, a median of the five respiratory rates may be calculated. In some embodiments, the median value may be compared to the predicted future data to determine a difference. In some embodiments, the method can comprise determining a difference between the predicted future data and a median value of the new data, and the difference may be indicative of an anomaly in the physiological condition of the user.

In some embodiments, the historical sensor data set may be based on an average. The average may be an average of input sensor data or a range of input sensor data. In some embodiments, the new data may not be compared to the historical sensor data set. In some embodiments, the new data may be compared to the historical sensor data set. In some embodiments, the predicted future data may not be based on an average of data values of the historical sensor data set.

The new data may be produced at a rate (e.g., a rate during a user's sleep session or use of the article of furniture). In some embodiments, the new data may be produced at least once per minute while a user is sleeping on the article of furniture (e.g., during a sleep session). In some embodiments, the new data may be produced at most once per minute while a user is sleeping on the article of furniture (e.g., during a sleep session). Without wishing to be bound by theory, the comparison of the predicted future data and a new data may enhance an accuracy of determining an anomaly of a physiological condition of a user.

As another example, provided herein are computer implemented methods for detecting a condition of a user of an article of furniture, the method comprising: determining, by a first computer algorithm, one or more sensor data signatures indicative of a physiological condition of the user via based on an input sensor data associated with a biological signal of the user, wherein the input sensor data is collected by a biological sensor while the user is sleeping on the article of furniture; classifying, by a second and different computer algorithm, the determined one or more sensor data signatures of (a) as normal one or more sensor data signatures or irregular one or more sensor data signatures; and filtering the irregular one or more sensor data signatures.

As another example, provided herein are computer-implemented methods for detecting an anomaly in a physiological condition of a user of an article of furniture, the method comprising: predicting a future data (e.g., future sensor data or analysis data thereof) based on a historical sensor data set associated with the user of an article of furniture, wherein the future data is indicative of a physiological condition of the user; and determining a difference between the predicted future data and a new data (e.g., new sensor data or analysis data thereof), wherein the difference is indicative of an anomaly in the physiological condition of the user.

In some embodiments, the article of furniture can comprise a bed device. A bed device may be a mattress, mattress cover, or combination thereof. In some embodiments, the article of furniture can be a mattress, a duvet, a pillow, or a cover thereof. In some embodiments, the future sensor data and/or analysis data thereof can be a future data. The terms “future sensor data” and “future data” may be used interchangeably herein. In some embodiments, the new sensor data and/or analysis data thereof can be a new data. The terms “new sensor data” and “new data” may be used interchangeably herein. The new data may be collected during a current use of the article of furniture (e.g., a current sleep session).

The historical sensor data set may comprise historical sensor data collected over previous uses of the article of furniture. In some embodiments, the historical sensor data set may be collected from one or more previous sleep sessions (e.g., previous sleep sessions of a user of the article of furniture). The historical sensor data set may comprise historical sensor data collected over a plurality of previous uses of the article of furniture. The plurality of previous uses may be consecutive previous uses. For example, a future sensor data or analysis data thereof beginning at Time 0 based on a historical sensor data set may comprise a historical sensor data set based on data from past uses of Time −5, Time −4, Time −3, Time −2, and Time −1 (e.g., consecutive uses or sleep sessions from 5 previous nights). The plurality of previous uses may be non-consecutive previous uses. For example, a future sensor data or analysis data thereof beginning at Time 0 based on a historical sensor data set may comprise a historical sensor data set based on data from past uses of Time −10, Time −8, Time −5, Time −2, and Time −1 (e.g., non-consecutive uses or sleep sessions from 5 previous nights).

In some embodiments, one or more sensor data signatures associated with the physiological condition of the user may be identified from the historical sensor data set. The future data can be based on one or more sensor data signatures. In some embodiments, the one or more sensor data signatures can comprise a range of sensor data values, a pattern of sensor data, or any combination thereof. The one or more sensor data signatures may comprise respiratory rate, breath cycle, heart rate, heart rate variability, vibration, or any combination thereof. In some embodiments, the one or more sensor data signatures may comprise respiratory rate, heart rate, and heart rate variability (HRV).

The physiological condition of the user may comprise a cardio-respiratory condition. For example, the cardio-respiratory condition can comprise one or more of heart attack, stroke, heart failure, arrhythmia, coronary artery disease, peripheral artery disease, aortic disease, congenital heart disease, chronic bronchitis, chronic obstructive pulmonary disease, or congestive heart failure. In some embodiments, the physiological condition can comprises a sleep disorder.

In some embodiments, predicting a future data (e.g., a future sensor data or analysis data thereof) can be based on a moving-average model. The moving-average model may be trained. In some embodiments, the moving-average model may be trained to assign a weight and/or influence to one or more recent data from the historical sensor data set. In some embodiments, the moving-average model is trained to assign a greater weight or influence to one or more recent data in the historical sensor data set as compared to one or more older data in the historical sensor data set. In some embodiments, the moving-average model is trained to assign a lesser weight or influence to one or more recent data in the historical sensor data set as compared to one or more older data in the historical sensor data set. In some embodiments, the moving-average model is trained to assign an equal weight or influence to one or more recent data in the historical sensor data set as compared to one or more older data in the historical sensor data set. The one or more recent data can comprise a number of uses of the article of furniture by the user. For example, the one or more recent data can comprise at least about 1 use, 2 uses, 3 uses, 4 uses, 5 uses, 6 uses, 7 uses, 8 uses, 9 uses, 10 uses, 15 uses, 20 uses, 25 uses, 30 uses, 35 uses, 40 uses, 50 uses, or greater than about 50 uses of the article of furniture by the user. The one or more recent data can comprise at most about 50 uses, 40 uses, 35 uses, 30 uses, 25 uses, 20 uses, 15 uses, 10 uses, 9 uses, 8 uses, 7 uses, 6 uses, 5 uses, 4 uses, 3 uses, 2 uses, 1 use, or less than 1 use of the article of furniture by the user. In some embodiments, the one or more recent data can comprise from about 1 use to about 20 uses of the article of furniture by the user. In some embodiments, the one or more recent data can comprise from about 1 use to about 2 uses, about 1 use to about 3 uses, about 1 use to about 4 uses, about 1 use to about 5 uses, about 1 use to about 6 uses, about 1 use to about 7 uses, about 1 use to about 8 uses, about 1 use to about 9 uses, about 1 use to about 10 uses, about 1 use to about 15 uses, about 1 use to about 20 uses, about 2 uses to about 3 uses, about 2 uses to about 4 uses, about 2 uses to about 5 uses, about 2 uses to about 6 uses, about 2 uses to about 7 uses, about 2 uses to about 8 uses, about 2 uses to about 9 uses, about 2 uses to about 10 uses, about 2 uses to about 15 uses, about 2 uses to about 20 uses, about 3 uses to about 4 uses, about 3 uses to about 5 uses, about 3 uses to about 6 uses, about 3 uses to about 7 uses, about 3 uses to about 8 uses, about 3 uses to about 9 uses, about 3 uses to about 10 uses, about 3 uses to about 15 uses, about 3 uses to about 20 uses, about 4 uses to about 5 uses, about 4 uses to about 6 uses, about 4 uses to about 7 uses, about 4 uses to about 8 uses, about 4 uses to about 9 uses, about 4 uses to about 10 uses, about 4 uses to about 15 uses, about 4 uses to about 20 uses, about 5 uses to about 6 uses, about 5 uses to about 7 uses, about 5 uses to about 8 uses, about 5 uses to about 9 uses, about 5 uses to about 10 uses, about 5 uses to about 15 uses, about 5 uses to about 20 uses, about 6 uses to about 7 uses, about 6 uses to about 8 uses, about 6 uses to about 9 uses, about 6 uses to about 10 uses, about 6 uses to about 15 uses, about 6 uses to about 20 uses, about 7 uses to about 8 uses, about 7 uses to about 9 uses, about 7 uses to about 10 uses, about 7 uses to about 15 uses, about 7 uses to about 20 uses, about 8 uses to about 9 uses, about 8 uses to about 10 uses, about 8 uses to about 15 uses, about 8 uses to about 20 uses, about 9 uses to about 10 uses, about 9 uses to about 15 uses, about 9 uses to about 20 uses, about 10 uses to about 15 uses, about 10 uses to about 20 uses, or about 15 uses to about 20 uses.

In some embodiments, the the moving-average model can comprise Exponential Moving Average (EMA), Simple Moving Average (SMA), Weighted Moving Average (WMA), Double Exponential Moving Average (DEMA), Triple Exponential Moving Average (TEMA), Smoothed Moving Average (SMMA), Adaptive Moving Average (AMA), Linear Weighted Moving Average (LWMA), Hull Moving Average (HMA), or a modification thereof, or any combination thereof. The moving-average model may comprise one or more Exponential Moving Average (EMA) models.

In some embodiments, the physiological condition may comprise a plurality of physiological conditions. The moving-average model may comprise a different moving-average model for each of the plurality of physiological conditions. The moving-average model may comprise the same moving-average model for each of the plurality of physiological conditions. In some embodiments, the new data and the future data may be different by a predetermined threshold metric. The predetermined threshold metric may indicate whether or not an anomaly has occurred when determining the difference between the predicted future data (e.g., future sensor data or analysis data thereof) and the new data (e.g., new sensor data or analysis data thereof). In some embodiments, the determined difference may be characterized by a deviation of the new data from the future data. For example, the determined difference may be characterized by deviation of the new data by at least about 1 standard deviation, at least about 2 standard deviations, at least about 2 standard deviations, at least about 3 standard deviations, at least about 4 standard deviations, at least about 5 standard deviations, or greater than about 5 standard deviations from the future data. As another example, the determined difference may be characterized by deviation of the new data by at most about 5 standard deviations, at most about 4 standard deviations, at most about 3 standard deviations, at most about 2 standard deviations, at most about 1 standard deviation, or less than about 1 standard deviation from the future data.

The future data (e.g., future sensor data or analysis data thereof) may be predicted any number of times based on the. historical sensor data set. In some embodiments, the future data (e.g., future sensor data or analysis data thereof) may be predicted at least about 1 time, 2 times, 3 times, 4 times, 5 times, 6 times, 7 times, 8 times, 9 times, 10 times, 15 times, 20 times, 30 times, 40 times, 50 times, or greater than about 50 times per minute. In some embodiments, the future data (e.g., future sensor data or analysis data thereof) may be predicted at most about 50 times, 40 times, 30 times, 20 times, 15 times, 10 times, 9 times, 8 times, 7 times, 6 times, 5 times, 4 times, 3 times, 2 times, 1 time, or less than about 1 time per minute.

Predicting the future data may be performed in real-time or substantially in real-time.

Predicting the future data may be performed in real-time or substantially in real-time relative to collection of the new sensor data. In some embodiments, the historical sensor data set and the new sensor data may be measured by the same sensor. In some embodiments, the historical sensor data set and the new sensor data may be measured by different same sensors. In some embodiments, the historical sensor data set and the new sensor data may be measured by one or more biological sensors. The one or more biological sensors may be one or more biological sensors described herein. In some embodiments, the biological sensor comprises a piezoelectric sensor. In some embodiments, the biological sensor comprises a temperature sensor.

A difference between the predicted future data (e.g., predicted future sensor data or analysis data thereof) and the new data (e.g., new sensor data or analysis data thereof) may be determined subsequent to determining that the user is sleeping on the article of furniture. A difference between the predicted future data (e.g., predicted future sensor data or analysis data thereof) and the new data (e.g., new sensor data or analysis data thereof) may be determined concurrently with the user sleeping on the article of furniture. In some embodiments, a digital report may be generated. The report may be based on the anomaly in the physiological condition of the user. In some embodiments, one or more digital reports may be generated. For example, if one or more anomalies are indicated by a difference between the predicted future data (e.g., predicted future sensor data or analysis data thereof) and the new data (e.g., new sensor data or analysis data thereof), one or more digital reports may be generated. In some embodiments, the one or more digital reports can be displayed. In some embodiments, one or more digital reports can be displayed on a graphical user interface (GUI) of a user device.

As another example, provided herein are computer-implement methods for detecting a condition of a user of an article of furniture, the method comprising: detecting a plurality of sensor data signatures from a sensor data set associated with a user of an article of furniture; identifying one or more acceptable quality sensor data signatures from the plurality of sensor data signatures via a signal quality filtering algorithm; and determining presence of the physiological condition of the user based on the one or more acceptable quality sensor data signatures identified.

In some embodiments, identifying the one or more acceptable quality sensor data signatures can comprise keeping high quality sensor data signatures from the plurality of sensor data signatures. In some embodiments, identifying the one or more acceptable quality sensor data signatures can comprise filtering out low quality sensor data signatures from the plurality of sensor data signatures. Identification of the one or more acceptable quality sensor data signatures may be performed by a signal quality processing model. The signal quality processing model may be based on signal-to-noise ratio detection, peak detection, spectral analysis-based detection, correlation-based detection, adaptive thresholding detection, or any combination thereof. In some embodiments, the signal quality processing model may be based on low-pass filter, high-pass filter, band-pass filter, band-reject filter, or any combination thereof.

Detection of one or more sensor data signatures from a sensor data set may be performed by a machine learning model. In some embodiments, the machine learning model may be a machine learning model described herein. In some embodiments, the machine learning model may be trained on clinical data. The clinical data can comprise electrocardiogram data. In some embodiments, the the machine learning model can comprise a deep learning model. The deep learning model may be a convolutional neural network (CNN). In some embodiments, detection of the one or more sensor data signatures and identification of the one or more acceptable quality sensor data signatures from the plurality of sensor data signatures may be performed by different computer algorithms. In some embodiments, detection of the one or more sensor data signatures and identification of the one or more acceptable quality sensor data signatures from the plurality of sensor data signatures may be performed by the same computer algorithm.

In some embodiments, one or more patterns (e.g., sensor data signature patterns) may be identified from the plurality of sensor data signatures. The one or more patterns may be associated with a physiological condition of the user. Determining a presence of the physiological condition of the user may be based on the one or more patterns. In some embodiments, a pattern may be classified as a normal pattern that is not associated with the physiological condition. In some embodiments, a pattern may be classified as an abnormal pattern that is associated with the physiological condition. Classifying of the one or more patterns may be based on a time-series filter. In some embodiments, classifying of the one or more patterns may be based, in part, on a time-series filter.

A presence of a physiological condition may be determined when a value (e.g., frequency) of a pattern meets a predetermined threshold (e.g., a predetermined threshold frequency value). The abnormal pattern may be characterized as a loading rate. In some embodiments, the predetermined threshold frequency value may be at least about 1%, at least about 2%, at least about 3%, at least about 4%, at least about 5%, at least about 6%, at least about 7%, at least about 8%, at least about 9%, at least about 10%, at least about 15%, at least about 20%, or greater than about 20%. In some embodiments, the predetermined threshold frequency value may be at most about 20%, at most about 15%, at most about 10%, at most about 9%, at most about 8%, at most about 7%, at most about 6%, at most about 5%, at most about 4%, at most about 3%, at most about 2%, at most about 1%, or less than about 1%. In some embodiments, a digital report may be generated based on a presence of the physiological condition (e.g., arrhythmia). In some embodiments, a digital report may be generated if the physiological condition (e.g., arrhythmia) is present. In some embodiments, a digital report may be generated if the physiological condition (e.g., arrhythmia) is not present. The digital report may be presented on an interface (e.g., a graphical user interface, GUI) described herein.

In some aspects, provided herein are systems comprising: a computer processor and a computer memory coupled thereto, wherein the computer memory comprises a machine executable code that, upon execution by the one or more computer processors, implements the methods described herein.

Detecting a condition (e.g., a breathing disturbance, cardiac disturbance, or combination thereof) using the methods described herein may detect a condition of one or more users with at least about 50% accuracy, at least about 60% accuracy, at least about 70% accuracy, at least about 75% accuracy, at least about 80% accuracy, at least about 85% accuracy, at least about 90% accuracy, at least about 91% accuracy, at least about 92% accuracy, at least about 93% accuracy, at least about 94% accuracy, at least about 95% accuracy, at least about 96% accuracy, at least about 97% accuracy, at least about 98% accuracy, at least about 99% accuracy, or greater than about 99% accuracy. Detecting a condition (e.g., a breathing disturbance, cardiac disturbance, or combination thereof) using the methods described herein may detect a condition of one or more users with at most about 99% accuracy, at most about 98% accuracy, at most about 97% accuracy, at most about 96% accuracy, at most about 95% accuracy, at most about 94% accuracy, at most about 93% accuracy, at most about 92% accuracy, at most about 91% accuracy, at most about 90% accuracy, at most about 85% accuracy, at most about 80% accuracy, at most about 75% accuracy, at most about 70% accuracy, at most about 60% accuracy, at most about 50% accuracy, or less than about 50% accuracy.

Breathing disturbance detection (e.g., apnea detection) using the methods described herein may detect a breathing disturbance of one or more users with at least about 50% accuracy, at least about 60% accuracy, at least about 70% accuracy, at least about 75% accuracy, at least about 80% accuracy, at least about 85% accuracy, at least about 90% accuracy, at least about 91% accuracy, at least about 92% accuracy, at least about 93% accuracy, at least about 94% accuracy, at least about 95% accuracy, at least about 96% accuracy, at least about 97% accuracy, at least about 98% accuracy, at least about 99% accuracy, or greater than about 99% accuracy. In some embodiments, breathing disturbance detection (e.g., apnea detection) using the methods described herein may detect a breathing disturbance of one or more users with at most about 99% accuracy, at most about 98% accuracy, at most about 97% accuracy, at most about 96% accuracy, at most about 95% accuracy, at most about 94% accuracy, at most about 93% accuracy, at most about 92% accuracy, at most about 91% accuracy, at most about 90% accuracy, at most about 85% accuracy, at most about 80% accuracy, at most about 75% accuracy, at most about 70% accuracy, at most about 60% accuracy, at most about 50% accuracy, or less than about 50% accuracy.

In some embodiments, breathing disturbance detection (e.g., apnea detection) using the methods described herein may detect a breathing disturbance of one or more users from about 50% accuracy to about 99% accuracy. In some embodiments, breathing disturbance detection (e.g., apnea detection) using the methods described herein may detect a breathing disturbance of one or more users from about 50% accuracy to about 60% accuracy, about 50% accuracy to about 70% accuracy, about 50% accuracy to about 75% accuracy, about 50% accuracy to about 80% accuracy, about 50% accuracy to about 85% accuracy, about 50% accuracy to about 90% accuracy, about 50% accuracy to about 92% accuracy, about 50% accuracy to about 94% accuracy, about 50% accuracy to about 96% accuracy, about 50% accuracy to about 98% accuracy, about 50% accuracy to about 99% accuracy, about 60% accuracy to about 70% accuracy, about 60% accuracy to about 75% accuracy, about 60% accuracy to about 80% accuracy, about 60% accuracy to about 85% accuracy, about 60% accuracy to about 90% accuracy, about 60% accuracy to about 92% accuracy, about 60% accuracy to about 94% accuracy, about 60% accuracy to about 96% accuracy, about 60% accuracy to about 98% accuracy, about 60% accuracy to about 99% accuracy, about 70% accuracy to about 75% accuracy, about 70% accuracy to about 80% accuracy, about 70% accuracy to about 85% accuracy, about 70% accuracy to about 90% accuracy, about 70% accuracy to about 92% accuracy, about 70% accuracy to about 94% accuracy, about 70% accuracy to about 96% accuracy, about 70% accuracy to about 98% accuracy, about 70% accuracy to about 99% accuracy, about 75% accuracy to about 80% accuracy, about 75% accuracy to about 85% accuracy, about 75% accuracy to about 90% accuracy, about 75% accuracy to about 92% accuracy, about 75% accuracy to about 94% accuracy, about 75% accuracy to about 96% accuracy, about 75% accuracy to about 98% accuracy, about 75% accuracy to about 99% accuracy, about 80% accuracy to about 85% accuracy, about 80% accuracy to about 90% accuracy, about 80% accuracy to about 92% accuracy, about 80% accuracy to about 94% accuracy, about 80% accuracy to about 96% accuracy, about 80% accuracy to about 98% accuracy, about 80% accuracy to about 99% accuracy, about 85% accuracy to about 90% accuracy, about 85% accuracy to about 92% accuracy, about 85% accuracy to about 94% accuracy, about 85% accuracy to about 96% accuracy, about 85% accuracy to about 98% accuracy, about 85% accuracy to about 99% accuracy, about 90% accuracy to about 92% accuracy, about 90% accuracy to about 94% accuracy, about 90% accuracy to about 96% accuracy, about 90% accuracy to about 98% accuracy, about 90% accuracy to about 99% accuracy, about 92% accuracy to about 94% accuracy, about 92% accuracy to about 96% accuracy, about 92% accuracy to about 98% accuracy, about 92% accuracy to about 99% accuracy, about 94% accuracy to about 96% accuracy, about 94% accuracy to about 98% accuracy, about 94% accuracy to about 99% accuracy, about 96% accuracy to about 98% accuracy, about 96% accuracy to about 99% accuracy, or about 98% accuracy to about 99% accuracy.

Cardiac disturbance detection (e.g., arrhythmia detection) using the methods described herein may detect a cardiac disturbance of one or more users with at least about 50% accuracy, at least about 60% accuracy, at least about 70% accuracy, at least about 75% accuracy, at least about 80% accuracy, at least about 85% accuracy, at least about 90% accuracy, at least about 91% accuracy, at least about 92% accuracy, at least about 93% accuracy, at least about 94% accuracy, at least about 95% accuracy, at least about 96% accuracy, at least about 97% accuracy, at least about 98% accuracy, at least about 99% accuracy, or greater than about 99% accuracy. In some embodiments, cardiac disturbance detection (e.g., arrhythmia detection) using the methods described herein may detect a cardiac disturbance of one or more users with at most about 99% accuracy, at most about 98% accuracy, at most about 97% accuracy, at most about 96% accuracy, at most about 95% accuracy, at most about 94% accuracy, at most about 93% accuracy, at most about 92% accuracy, at most about 91% accuracy, at most about 90% accuracy, at most about 85% accuracy, at most about 80% accuracy, at most about 75% accuracy, at most about 70% accuracy, at most about 60% accuracy, at most about 50% accuracy, or less than about 50% accuracy.

In some embodiments, cardiac disturbance detection (e.g., arrhythmia detection) using the methods described herein may detect a cardiac disturbance of one or more users from about 50% accuracy to about 99% accuracy. In some embodiments, cardiac disturbance detection (e.g., arrhythmia detection) using the methods described herein may detect a cardiac disturbance of one or more users from about 50% accuracy to about 60% accuracy, about 50% accuracy to about 70% accuracy, about 50% accuracy to about 75% accuracy, about 50% accuracy to about 80% accuracy, about 50% accuracy to about 85% accuracy, about 50% accuracy to about 90% accuracy, about 50% accuracy to about 92% accuracy, about 50% accuracy to about 94% accuracy, about 50% accuracy to about 96% accuracy, about 50% accuracy to about 98% accuracy, about 50% accuracy to about 99% accuracy, about 60% accuracy to about 70% accuracy, about 60% accuracy to about 75% accuracy, about 60% accuracy to about 80% accuracy, about 60% accuracy to about 85% accuracy, about 60% accuracy to about 90% accuracy, about 60% accuracy to about 92% accuracy, about 60% accuracy to about 94% accuracy, about 60% accuracy to about 96% accuracy, about 60% accuracy to about 98% accuracy, about 60% accuracy to about 99% accuracy, about 70% accuracy to about 75% accuracy, about 70% accuracy to about 80% accuracy, about 70% accuracy to about 85% accuracy, about 70% accuracy to about 90% accuracy, about 70% accuracy to about 92% accuracy, about 70% accuracy to about 94% accuracy, about 70% accuracy to about 96% accuracy, about 70% accuracy to about 98% accuracy, about 70% accuracy to about 99% accuracy, about 75% accuracy to about 80% accuracy, about 75% accuracy to about 85% accuracy, about 75% accuracy to about 90% accuracy, about 75% accuracy to about 92% accuracy, about 75% accuracy to about 94% accuracy, about 75% accuracy to about 96% accuracy, about 75% accuracy to about 98% accuracy, about 75% accuracy to about 99% accuracy, about 80% accuracy to about 85% accuracy, about 80% accuracy to about 90% accuracy, about 80% accuracy to about 92% accuracy, about 80% accuracy to about 94% accuracy, about 80% accuracy to about 96% accuracy, about 80% accuracy to about 98% accuracy, about 80% accuracy to about 99% accuracy, about 85% accuracy to about 90% accuracy, about 85% accuracy to about 92% accuracy, about 85% accuracy to about 94% accuracy, about 85% accuracy to about 96% accuracy, about 85% accuracy to about 98% accuracy, about 85% accuracy to about 99% accuracy, about 90% accuracy to about 92% accuracy, about 90% accuracy to about 94% accuracy, about 90% accuracy to about 96% accuracy, about 90% accuracy to about 98% accuracy, about 90% accuracy to about 99% accuracy, about 92% accuracy to about 94% accuracy, about 92% accuracy to about 96% accuracy, about 92% accuracy to about 98% accuracy, about 92% accuracy to about 99% accuracy, about 94% accuracy to about 96% accuracy, about 94% accuracy to about 98% accuracy, about 94% accuracy to about 99% accuracy, about 96% accuracy to about 98% accuracy, about 96% accuracy to about 99% accuracy, or about 98% accuracy to about 99% accuracy.

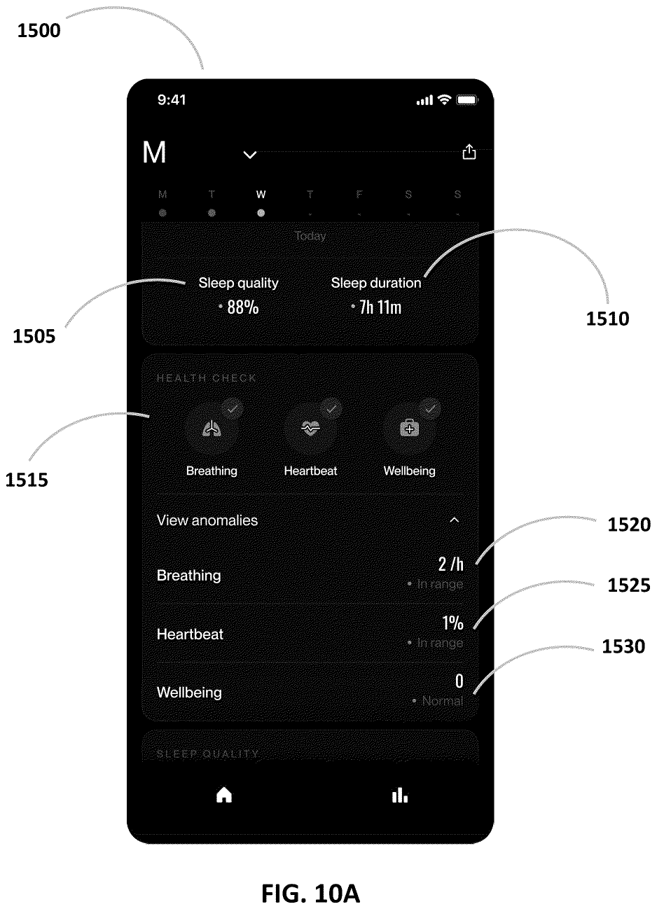

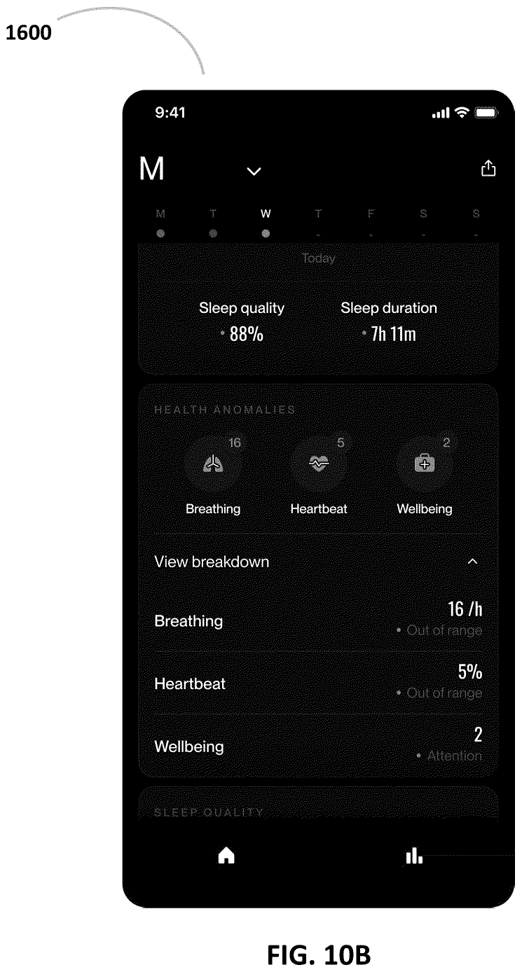

A representation of a user interface 1500 is shown in FIG. 10A. The interface 1500 shows a percentage rating of sleep quality 1505 as well as a duration of time for sleep duration 1510. An area of the interface depicts icons for breathing, heartbeat, and wellbeing 1515. The checkmark next to each icon can indicate no anomalies (e.g., the computer algorithm did not find a value for a data signature differed significantly from an expected, or historical value). The checkmark next to each icon can indicate a number of anomalies below a designated threshold (e.g., the computer algorithm found one or more values for a data signature that differed from an expected, or historical value). The user interface 1500 also can comprise values for each health component (e.g., respiratory, cardiac, and/or wellbeing component). For example, the breathing area may show a number of breathing anomalies per hour (1520). A breathing anomaly may be a breath gap as described herein. The breathing anomaly may be determined and/confirmed by a first computer algorithm and/or second computer algorithm as described herein.

A checkmark next to the breathing icon in the area 1515 can indicate that the number of breathing anomalies was within a threshold range for that sleep session. For example, there may be an average of 2 breathing anomalies per hour 1520 and that can be within a threshold range. In some embodiments, a range of breathing anomalies can be from about 0 anomalies per hour (anomalies/hour) to about 20 anomalies per hour (anomalies/hour). In some embodiments, a range of breathing anomalies can be from about 0 anomalies per hour (anomalies/hour) to about 15 anomalies per hour (anomalies/hour). In some embodiments, a range of breathing anomalies can be from about 0 anomalies per hour (anomalies/hour) to about 14 anomalies per hour (anomalies/hour). In some embodiments, a range of breathing anomalies can be from about 0 anomalies per hour (anomalies/hour) to about 1 anomaly per hour (anomaly/hour), about 0 anomalies per hour (anomalies/hour) to about 2 anomalies per hour (anomalies/hour), about 0 anomalies per hour (anomalies/hour) to about 3 anomalies per hour (anomalies/hour), about 0 anomalies per hour (anomalies/hour) to about 4 anomalies per hour (anomalies/hour), about 0 anomalies per hour (anomalies/hour) to about 5 anomalies per hour (anomalies/hour), about 0 anomalies per hour (anomalies/hour) to about 6 anomalies per hour (anomalies/hour), about 0 anomalies per hour (anomalies/hour) to about 7 anomalies per hour (anomalies/hour), about 0 anomalies per hour (anomalies/hour) to about 8 anomalies per hour (anomalies/hour), about 0 anomalies per hour (anomalies/hour) to about 9 anomalies per hour (anomalies/hour), about 0 anomalies per hour (anomalies/hour) to about 10 anomalies per hour (anomalies/hour), about 0 anomalies per hour (anomalies/hour) to about 20 anomalies per hour (anomalies/hour), about 1 anomaly per hour (anomaly/hour) to about 2 anomalies per hour (anomalies/hour), about 1 anomaly per hour (anomaly/hour) to about 3 anomalies per hour (anomalies/hour), about 1 anomaly per hour (anomaly/hour) to about 4 anomalies per hour (anomalies/hour), about 1 anomaly per hour (anomaly/hour) to about 5 anomalies per hour (anomalies/hour), about 1 anomaly per hour (anomaly/hour) to about 6 anomalies per hour (anomalies/hour), about 1 anomaly per hour (anomaly/hour) to about 7 anomalies per hour (anomalies/hour), about 1 anomaly per hour (anomaly/hour) to about 8 anomalies per hour (anomalies/hour), about 1 anomaly per hour (anomaly/hour) to about 9 anomalies per hour (anomalies/hour), about 1 anomaly per hour (anomaly/hour) to about 10 anomalies per hour (anomalies/hour), about 1 anomaly per hour (anomaly/hour) to about 20 anomalies per hour (anomalies/hour), about 2 anomalies per hour (anomalies/hour) to about 3 anomalies per hour (anomalies/hour), about 2 anomalies per hour (anomalies/hour) to about 4 anomalies per hour (anomalies/hour), about 2 anomalies per hour (anomalies/hour) to about 5 anomalies per hour (anomalies/hour), about 2 anomalies per hour (anomalies/hour) to about 6 anomalies per hour (anomalies/hour), about 2 anomalies per hour (anomalies/hour) to about 7 anomalies per hour (anomalies/hour), about 2 anomalies per hour (anomalies/hour) to about 8 anomalies per hour (anomalies/hour), about 2 anomalies per hour (anomalies/hour) to about 9 anomalies per hour (anomalies/hour), about 2 anomalies per hour (anomalies/hour) to about 10 anomalies per hour (anomalies/hour), about 2 anomalies per hour (anomalies/hour) to about 20 anomalies per hour (anomalies/hour), about 3 anomalies per hour (anomalies/hour) to about 4 anomalies per hour (anomalies/hour), about 3 anomalies per hour (anomalies/hour) to about 5 anomalies per hour (anomalies/hour), about 3 anomalies per hour (anomalies/hour) to about 6 anomalies per hour (anomalies/hour), about 3 anomalies per hour (anomalies/hour) to about 7 anomalies per hour (anomalies/hour), about 3 anomalies per hour (anomalies/hour) to about 8 anomalies per hour (anomalies/hour), about 3 anomalies per hour (anomalies/hour) to about 9 anomalies per hour (anomalies/hour), about 3 anomalies per hour (anomalies/hour) to about 10 anomalies per hour (anomalies/hour), about 3 anomalies per hour (anomalies/hour) to about 20 anomalies per hour (anomalies/hour), about 4 anomalies per hour (anomalies/hour) to about 5 anomalies per hour (anomalies/hour), about 4 anomalies per hour (anomalies/hour) to about 6 anomalies per hour (anomalies/hour), about 4 anomalies per hour (anomalies/hour) to about 7 anomalies per hour (anomalies/hour), about 4 anomalies per hour (anomalies/hour) to about 8 anomalies per hour (anomalies/hour), about 4 anomalies per hour (anomalies/hour) to about 9 anomalies per hour (anomalies/hour), about 4 anomalies per hour (anomalies/hour) to about 10 anomalies per hour (anomalies/hour), about 4 anomalies per hour (anomalies/hour) to about 20 anomalies per hour (anomalies/hour), about 5 anomalies per hour (anomalies/hour) to about 6 anomalies per hour (anomalies/hour), about 5 anomalies per hour (anomalies/hour) to about 7 anomalies per hour (anomalies/hour), about 5 anomalies per hour (anomalies/hour) to about 8 anomalies per hour (anomalies/hour), about 5 anomalies per hour (anomalies/hour) to about 9 anomalies per hour (anomalies/hour), about 5 anomalies per hour (anomalies/hour) to about 10 anomalies per hour (anomalies/hour), about 5 anomalies per hour (anomalies/hour) to about 20 anomalies per hour (anomalies/hour), about 6 anomalies per hour (anomalies/hour) to about 7 anomalies per hour (anomalies/hour), about 6 anomalies per hour (anomalies/hour) to about 8 anomalies per hour (anomalies/hour), about 6 anomalies per hour (anomalies/hour) to about 9 anomalies per hour (anomalies/hour), about 6 anomalies per hour (anomalies/hour) to about 10 anomalies per hour (anomalies/hour), about 6 anomalies per hour (anomalies/hour) to about 20 anomalies per hour (anomalies/hour), about 7 anomalies per hour (anomalies/hour) to about 8 anomalies per hour (anomalies/hour), about 7 anomalies per hour (anomalies/hour) to about 9 anomalies per hour (anomalies/hour), about 7 anomalies per hour (anomalies/hour) to about 10 anomalies per hour (anomalies/hour), about 7 anomalies per hour (anomalies/hour) to about 20 anomalies per hour (anomalies/hour), about 8 anomalies per hour (anomalies/hour) to about 9 anomalies per hour (anomalies/hour), about 8 anomalies per hour (anomalies/hour) to about 10 anomalies per hour (anomalies/hour), about 8 anomalies per hour (anomalies/hour) to about 20 anomalies per hour (anomalies/hour), about 9 anomalies per hour (anomalies/hour) to about 10 anomalies per hour (anomalies/hour), about 9 anomalies per hour (anomalies/hour) to about 20 anomalies per hour (anomalies/hour), or about 10 anomalies per hour (anomalies/hour) to about 20 anomalies per hour (anomalies/hour).

A checkmark next to the heart icon can indicate that the number of cardiac anomalies was within a threshold range (e.g., loading threshold) for that sleep session. A loading rate can comprise an alert rate that may align with an expected prevalence of irregular heartbeats within a general population. For example, there may be an average of 1% of heart anomalies over a sleep session 1525 and that can be within a threshold range. In some embodiments, a loading threshold for cardiac disturbance detection (e.g., arrhythmia detection) may be at most 5%. In some embodiments, a loading threshold for cardiac disturbance detection (e.g., arrhythmia detection) may be at most 4%. In some embodiments, a loading threshold for cardiac disturbance detection (e.g., arrhythmia detection) may be at most 3%. In some embodiments, a loading threshold for cardiac disturbance detection (e.g., arrhythmia detection) may be at most 2%. In some embodiments, a loading threshold for cardiac disturbance detection (e.g., arrhythmia detection) may be from about 0% to about 5%. In some embodiments, a loading threshold for cardiac disturbance detection (e.g., arrhythmia detection) may be from about 0% to about 0.5%, about 0% to about 1%, about 0% to about 1.5%, about 0% to about 2%, about 0% to about 2.5%, about 0% to about 3%, about 0% to about 3.5%, about 0% to about 4%, about 0% to about 4.5%, about 0% to about 5%, about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5%. In some embodiments, a loading threshold for cardiac disturbance detection (e.g., arrhythmia detection) may be at least 5%. In some embodiments, a loading threshold for cardiac disturbance detection (e.g., arrhythmia detection) may be greater than about 5%.

A checkmark next to the wellbeing icon in the area 1515 can indicate that zero health anomalies were detected using the one or more computer algorithms described herein. A checkmark next to the wellbeing icon in the area 1515 can indicate that less than about 5, less than about 4, less than about 3, less than about 2, or less than about 1 health anomalies were detected using the one or more computer algorithms described herein. For example, user interface 1500 shows 0 anomalies detected in the wellbeing area 1530. The computer algorithm may have determined that one or more data signatures values of the user (e.g., respiratory rate, heart rate, heart rate variability, or any combination thereof) did not fall out of an expected range for that user. The computer algorithm may have determined that one or more data signatures values of the user (e.g., respiratory rate, heart rate, heart rate variability, or any combination thereof) did not deviate from a historical value for that user.

The user interface 1600 as shown in FIG. 10B provides an embodiment of the output of the methods and systems described herein. For example, the user interface 1600 shows a number next to the each icon for breathing, heartbeat, and wellbeing 1515 which indicates one or more anomalies. These can be advantageous for a user to identify or detect a condition. For example, a user may see a number of breathing anomalies from the user interface 1600 and identify a breathing disturbance (e.g., apnea event, snoring, or combination thereof). As another example, a user may see a number of cardiac anomalies from the user interface 1600 and identify a cardiac disturbance (e.g., arrhythmia). As another example, a user may see a number of wellbeing anomalies from the user interface 1600 and identify an illness or physiological stressor described herein. In some embodiments, the user interface 1500, 1600 can be configured to show data from a user over a single sleep session. In some embodiments, the user interface 1500, 1600 can be configured to show data from a user over one or more sleep sessions. In some embodiments, the user interface 1500, 1600 can be configured to show data from a user over at least about 1 sleep session, at least about 2 sleep sessions, at least about 3 sleep sessions, at least about 4 sleep sessions, at least about 5 sleep sessions, at least about 6 sleep sessions, at least about 7 sleep sessions, at least about 8 sleep sessions, at least about 9 sleep sessions, at least about 10 sleep sessions, at least about 11 sleep sessions, at least about 12 sleep sessions, at least about 13 sleep sessions, at least about 14 sleep sessions, at least about 15 sleep sessions, at least about 20 sleep sessions, at least about 25 sleep sessions, at least about 30 sleep sessions, at least about 40 sleep sessions, at least about 50 sleep sessions, at least about 60 sleep sessions, at least about 70 sleep sessions, at least about 80 sleep sessions, at least about 90 sleep sessions, at least about 100 sleep sessions, or greater than about 100 sleep sessions. Without wishing to be bound by theory, monitoring of the user's health over one or more sleep sessions using the methods described herein can provide a user with valuable information on their longitudinal health and wellbeing. The results may improve a user's sleep quality and/or sleep duration.

### Algorithms

In some embodiments, any decision or action of the controller (or processor as used interchangeably herein) as provided herein can be decided (or generated) by analyzing data based on one or more computer implemented models.

In some embodiments, the computer implemented model is a classifier as provided herein can be utilized to process (e.g., analyze) one or more of (i) a user signal from the sensing device (e.g., a user electromagnetic radiation signal), (ii) a reference signal from the reference device (e.g., a reference electromagnetic radiation signal), (iii) a contact sensor signal from a contact sensor (e.g., that is disposed in an article of furniture), (iv) data from outside sources, (v) data from user input, (vi) data from smart home (e.g., from one or more additional smart devices in smart home), or a combination thereof, to determine at least one physiological feature of the subject.

In some embodiments, the classifier can be trained based on past data associated with the subject or a cohort of subjects. The past data can comprise one or more of (i)-(vi) as provided herein. The past data can comprise data collected at least or at most about 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, 15 months, 18 months, 21 months, or 24 months prior to use of the classifier to determine the at least one physiological feature of a target subject. In some cases, the classifier can be continuously trained with new data comprising one or more of (i)-(vi). The frequency of continuous training (or update) of the classifier can be at least once per day, week (or multiple weeks), month (or multiple months), year (or multiple years), etc.

In some embodiments, the classifier can be trained based on data that is not collected by the sensors as provided herein (e.g., non-contact sensors of the sensing device, contact sensors of the article of furniture, etc.). In some embodiments, the classifier can be trained on a reference data. In some cases, the reference data can comprise clinical data (e.g., thermal imaging data and analysis thereof from experimental studies or clinical studies) collected from a cohort of individuals. In some cases, the reference data can be an artificial data that is not collected from any specific individual. For example the reference data can comprise predicted or hypothetical data of one or more biological signals (e.g., to be utilized as pseudo-ground truth data). In some cases, the reference data can be utilized as a ground truth data.

In some embodiments, the classifier as provided herein can be trained by applying machine learning algorithms (e.g., deep learning algorithms, clustering algorithms, forest based models, regression models, classifier models, etc.) on the control data as disclosed herein as a training dataset. Non-limiting examples of machine learning algorithms for training a classifier can include supervised learning, unsupervised learning, semi-supervised learning, reinforcement learning, self-learning, feature learning, anomaly detection, association rules, etc. In some cases, the classifier can be trained by using one or more learning models on such training dataset. Non-limiting examples of learning models can include artificial neural networks (e.g., convolutional neural networks, U-net architecture neural network, etc.), backpropagation, boosting, decision trees, support vector machines, regression analysis, Bayesian networks, genetic algorithms, kernel estimators, conditional random field, random forest, ensembles of classifiers, minimum complexity machines (MCM), probably approximately correct learning (PACT), etc.

In some embodiments, the classifier can continue to be trained by applying machine learning algorithms on the control data as well as any new input data (e.g., the current sensing data detected and generated substantially in real-time or within the short span of time as provided herein) as a training dataset.

In some embodiments, the computer implemented model (e.g., for determining the physiological condition of the subject) can be an unsupervised, semi-supervised or self-supervised model. Models such as these may not require the output of the model to be used as the output used for downstream applications or modeling while other embodiments may. Non-limiting methods include transformer models, BERT models, ERNIE models, autoencoders, variational autoencoders, generative adversarial networks, ladder network.

In some embodiments, the computer implemented model (e.g., for determining the physiological condition of the subject) can be a generative model. Non-limiting examples may include variational autoencoders, encoders, deconvolution encoders, generative adversarial networks, transformer models.

In some embodiments, the computer implemented model (e.g., for determining the physiological condition of the subject) can be a multimodal artificial intelligence model. Examples of such models include any model which takes in features of different types or modalities, such as but not limited to; image, video, text, sensor, spatial, tabular, genetic, clinical, temporal, discrete or continuous. Non-limiting examples of such methods include large language models (LLM), diffusion models, generative pretrained transformer models.

In some embodiments, the computer implemented model (e.g., for determining the physiological condition of the subject) can be trained in a transfer learning method. In such embodiments, the model may first be trained on a plurality of data that is only related to the desired task that the model through the data modality where the plurality of training data may contain information relevant to the desired task of the model in its most basic form. The computer implemented model may then be used on a different plurality of data that is specific to the desired task. Examples of such training methods may, in a non-limiting way, include training a model for disease detection through infrared data by first training the model on data collected by a sensor in various settings (e.g., outdoors, indoors, in a car, in different climates); the method would then train the model a second time, while retaining the trained parameters of the first training step, on infrared data specific to disease detection. The benefit of such training methods can be one or both of: (1) they can allow for a model to learn the larger distribution of the modalities being input to the model and (2) they can allow a model to be trained using a smaller set of data specific to the desired task without suffering from undergeneralization.

In some embodiments, the computer implemented model (e.g., for determining the physiological condition of the subject) can utilize user input. The user input is not limited to input methods of the device or devices disclosed herein and may include, but are not limited to, smartphones, computers, web-interface, app-enabled devices, user made devices, third party devices, third party apps. In some cases, such device can comprise a graphical user interface (GUI) configured to allow the user to provide the input. Such user input may be the result of a prompt from the input method. User input may come from a source such as, but not limited to, a user of the devices disclosed herein, or may be other users such as a friend, family member, a physician or a professional of any kind. For example, the input can comprise how well the user slept (e.g., or how the user perceives the sleep quality of one or more past nights) or how the user thinks about the user's health.

In some embodiments, the computer implemented method (e.g., for determining the physiological condition of the subject) can use data collected about the environment or other contextual information not specific to the user. Examples of such data sources can include, but are not limited to, daylight cycle, time of year, household temperature, humidity, air quality, general stressor (e.g., news stories), noise level, allergen levels, indoor light levels, traffic levels in the household etc.

In some embodiments, the computer implemented model (e.g., for determining the physiological condition of the subject) can have access to data provided by other devices such as, but not limited to smart home devices. Examples of such devices may include, but are not limited to, thermostats, humidity sensors, lights, devices connected to the internet of things, devices connected by z-wave protocol, matter protocol, devices connected by zigbee protocol, devices connected physically, devices connected through internet connection, devices on a mesh network, window controllers, shade controllers, blind controllers, doorbells, security cameras, door locks, door sensors, window sensors, plugs. In some cases, the computer implemented model may interface with smart home apps such as Phillips Hue, Smart assistant, Amazon Alexa, Samsung SmartThings, Google Nest, Smart Home Manager.

In some embodiments, the computer implemented model (e.g., for determining the physiological condition of the subject) can have access to information available on the internet. Examples of such information include, but aren't limited to, social media data, internet use data, purchasing habits.

### Computer Systems

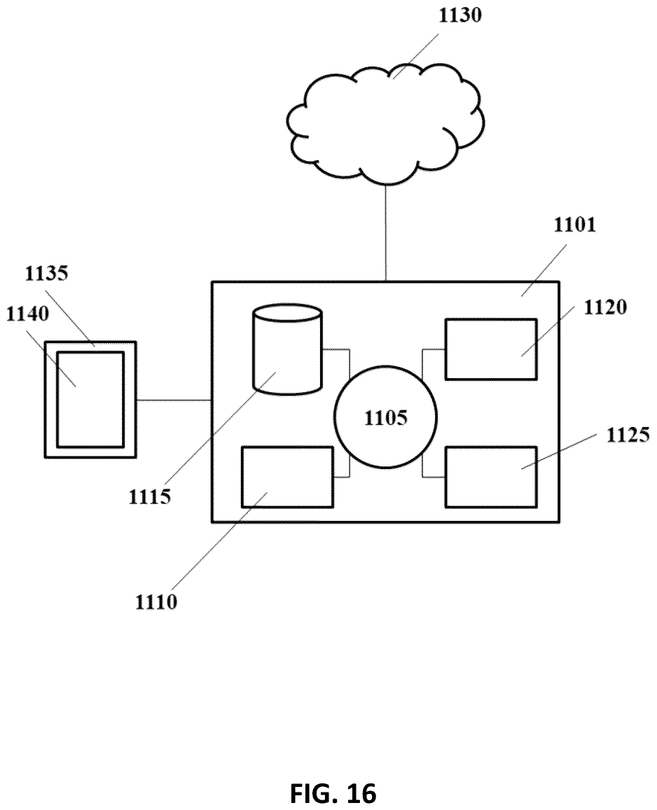

The present disclosure provides computer systems that are programmed to implement methods of the disclosure. FIG. 16 shows a computer system 1101 that is programmed or otherwise configured to direct operation of the system of the present disclosure (e.g., the sensing device, the reference device, the article of furniture, other devices, computer processor, etc.). In some embodiments, the computer system 1101 can be programmed or otherwise configured to monitor, project and/or regulate a parameter (e.g., temperature) of a bed device, a user, or both in accordance with any of the methods provided herein. The computer system 1101 can regulate various aspects of the methods as provided herein, such as, for example, directing the regulator to adjust at least one sleep-related parameter for the bed device to enhance sleep of the user, directing the projector to project the optical pattern to the wall or the ceiling adjacent the bed device, or both. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 (e.g., the projector as provided herein) that comprises a user interface (UI) 1140 for providing, for example, at least one of (a) status information for the bed device, local environment, or both or (b) at least one control option to direct operation of the sleep enhancement device. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, assist in comparison of the sensing data and control data as provided herein. The algorithm can, for example, analyze data collected from the bed device, the user, or both, for implementing any of the methods provided herein.

### EXAMPLES

### Example 1: Detection of User Signal and Reference Signal

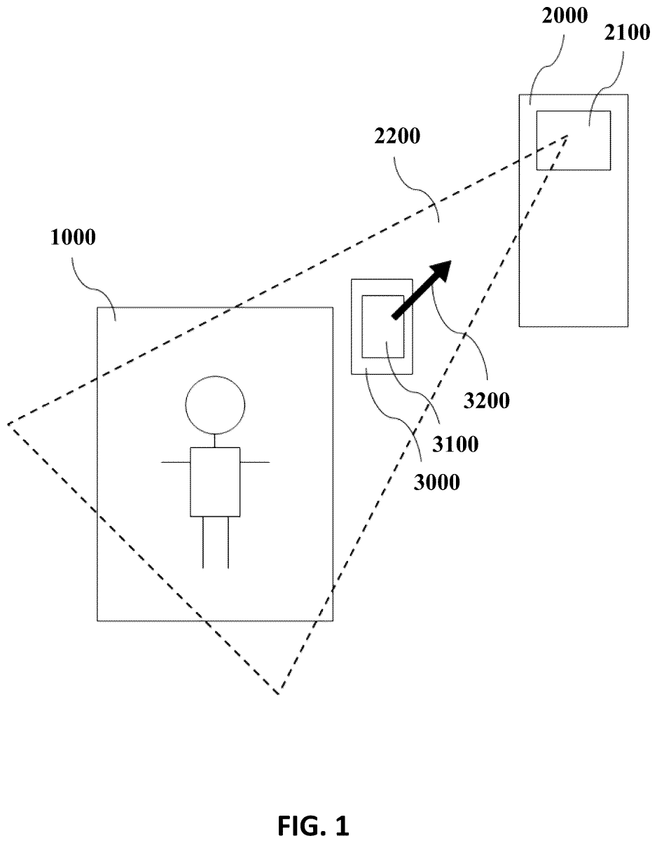

FIG. 1 schematically illustrates an example of the systems and methods of the present disclosure. The system can be utilized to detect a physiological feature of a subject on an article of furniture 1000 (e.g., a bed device). The system can comprise a sensing device 2000 comprising a housing and at least one sensor 2100 in sensing communication with an outer environment of the housing, such that the sensing area (e.g., field of view) 2200 of the at least one sensor 2100 can cover at least a portion of the body of the subject on the article of furniture (e.g., substantially whole body). The at least one sensor 2100 can be configured to detect a user signal that is reflected or emitted by at least a portion of the body of the subject. The system can further comprise a reference device 3000 comprising at least one signal source 3100 configured to output a reference signal 3200. The reference signal 3200 can be provided within the sensing area 2200, to be detectable by the at least one sensor 2100. In some cases, the reference signal 3200 can be detected and utilized as a baseline or a control signal when analyzing the detected user signal.

In some embodiments, the at least one sensor 2100 can comprise an infrared sensor configured to detect infrared light reflected or emitted by the least the portion of the body of the subject. The reference signal 3200 can be a reference infrared light having a defined wavelength or wavelength range, and the at least one signal source 3100 can comprise an optical source configured to emit the reference infrared light.

In some embodiments, the at least one sensor 2100 can comprise an acoustic or ultrasound sensor configured to detect acoustic or ultrasound signal from at least the portion of the body of the subject. The reference signal 3200 can be a reference acoustic or ultrasound signal having defined wavelength or frequency, and the at least one signal source 3100 can comprise a speaker or alike to provide the reference acoustic or ultrasound signal.

### Example 2: Detection of Non-Contact Sensor Signal and Contact-Sensor Signal

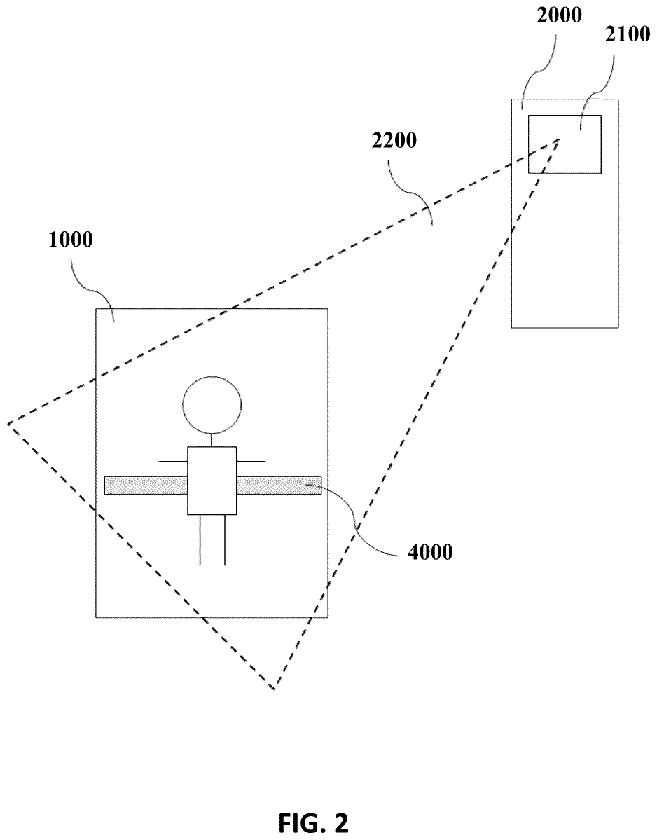

FIG. 2 schematically illustrates an example of the systems and methods of the present disclosure. The system can be utilized to detect a physiological feature of a subject on an article of furniture 1000 (e.g., a bed device). The system can comprise a sensing device 2000 comprising a housing and at least one sensor 2100 in sensing communication with an outer environment of the housing, such that the sensing area (e.g., field of view) 2200 of the at least one sensor 2100 can cover at least a portion of the body of the subject on the article of furniture (e.g., substantially whole body). The at least one sensor 2100 can be configured to detect a user signal that is reflected or emitted by at least a portion of the body of the subject. The user signal can be an electromagnetic signal (e.g., a non-contact sensor signal), and the at least one sensor 2100 can be a non-contact sensor configured to detect the electromagnetic signal from the user. The system can further comprise a contact sensor 4000 configured to detect a biological signal of the user (e.g., a contact sensor signal)while in contact (direct or indirect contact) with the user. For example, the contact sensor 4000 can comprise a capacitance sensor, a pressure sensor (e.g., a piezoelectric sensor), a temperature sensor, or a combination thereof. In some cases, the contact sensor 4000 can be part of the article of furniture 1000, e.g., part of a mattress or a mattress cover.

In some embodiments, the non-contact sensor signal of the user (e.g., infrared light) from the sensor 2100 and the contact-sensor signal of the user can be analyzed in combination or relative to one another, to determine one or more physiological features (e.g., health condition, such as a disease) of the subject.

### Example 3: Projection of Information With Capsule Projector

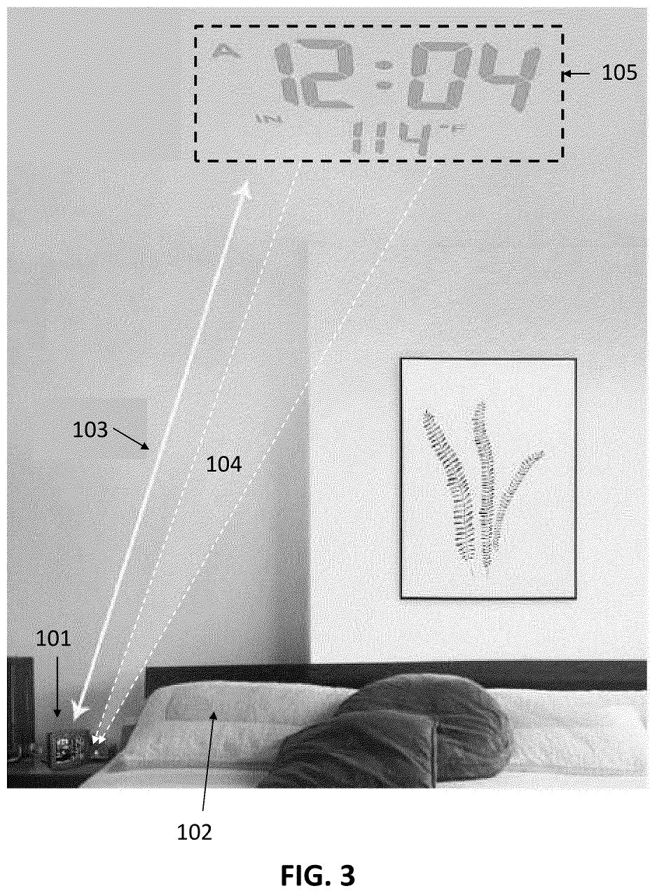

FIG. 3 illustrates an example system of the present disclosure. The system can comprise the capsule 101, situated next to a bed 102. The capsule 101 comprises a projector that generates a projection 104 on a surface, such as a ceiling. The projection presents multifaceted information 105, comprising one or more of time, temperature, environmental metrics, and sleep data. The information projected offers an interactive user interface, allowing control of a regulator (e.g., for adjusting at least one sleep-related parameter for the bed device to enhance sleep of the user) directly from the bed. The surface is at a distance 103 from the capsule 101, indicative of the capsule's robust projection capabilities over large distances. Accordingly, the system can deliver a user-friendly experience that enhances the sleep environment beyond basic sleep tracking.

### Example 4: Views of Capsule Projector and Projections

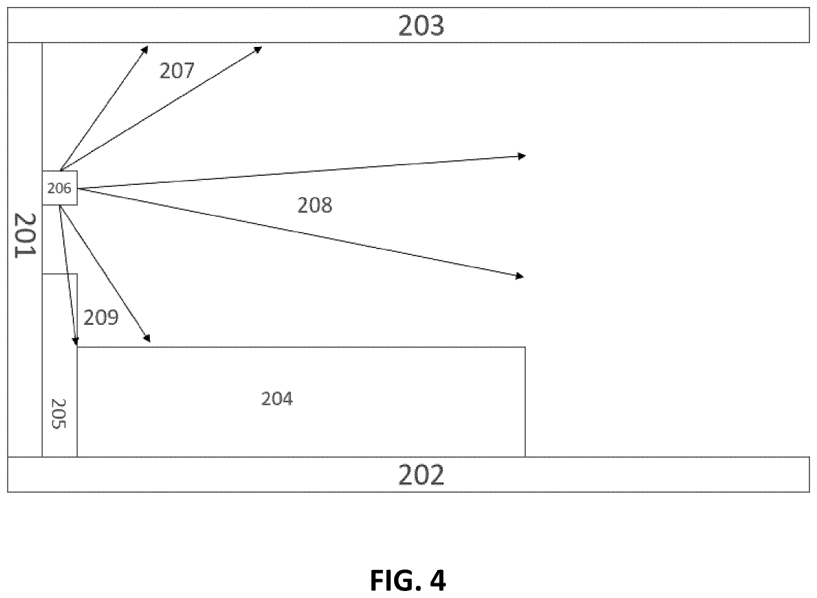

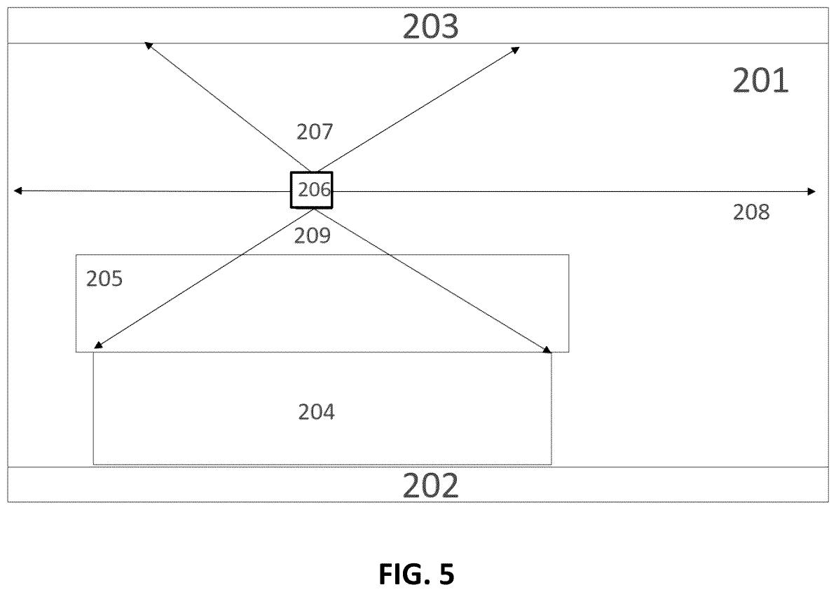

FIG. 4 and FIG. 5 illustrate side and front views of a system, represented as a schematic example system of the present disclosure. The system can comprise a capsule 206, mounted on a wall 201 above a headboard 205. The capsule comprises one or more sensors (e.g., a video camera, a thermal camera and/or an infrared camera). The placement of the capsule 206 above the headboard 205 permits the one or more sensors to attain fields of view 208 and 209 over a bed 204, located on a floor 202, and/or a surrounding environment. The capsule 206 comprises a projector that generates a projection on a surface, such as a ceiling 203, coming within the projector's field of view 207. The projection presents multifaceted information comprising one or more of time, temperature, environmental metrics, and sleep data.

### Example 5: Geometry of Capsule Projector

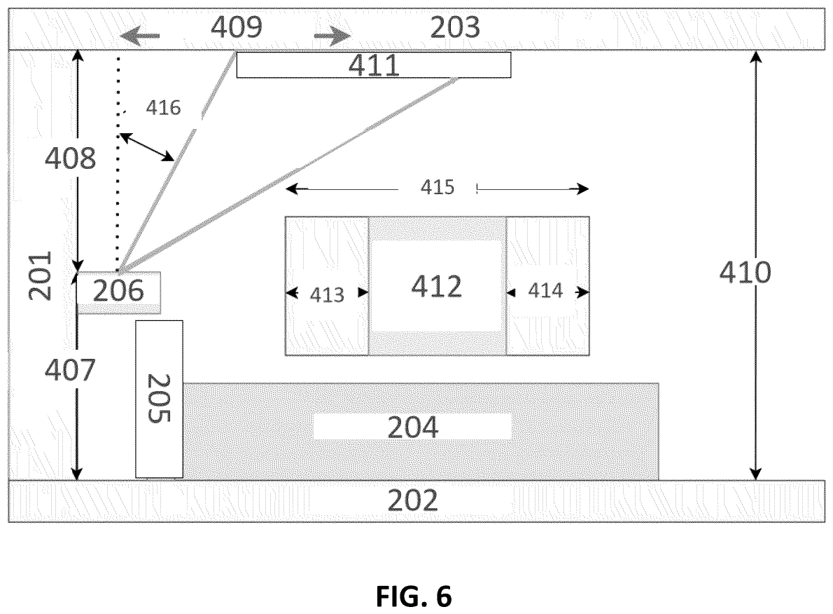

FIG. 6 illustrates a side view of a system represented as a schematic example system of the present disclosure. The system can comprise a capsule 206, mounted on a wall 201 above a headboard 205. The FIG. 4 illustrates an example of the system's geometry. The capsule comprises one or more sensors (e.g., a video camera, a thermal camera and/or an infrared camera). As depicted, an example projector's distance 407 from a floor 202 to the capsule 206 is about 1.42 m (56 in). An example distance 408 from the capsule 206 to a ceiling 203 is about 1.32 m (52 in). An example angle 416 between a reference vertical line and the projector's field of view is about 30 degrees, with an about 100% offset. An example projected offset 409 is about 1 m. An example width 411 of a projection on the ceiling is about 90.3 cm (35.5 in). An example image width 415 is also about 90.3 cm (35.5 in for a 16:9 aspect ratio), with an example throw ratio of about 1.46 (i.e., 1.32 m/0.903 m). An example ceiling height 410 is about 2.74 m (9 ft). An example UI image size 412 is a 50.8 cm by 50.8 cm square (20 in by 20 in), with example side views 413 and 414 of each measuring 19.75 cm (7.75 in). The capsule 206 comprises a projector that generates a projection on a surface, such as a ceiling 203. The projection presents multifaceted information comprising one or more of time, temperature, environmental metrics, and sleep data.

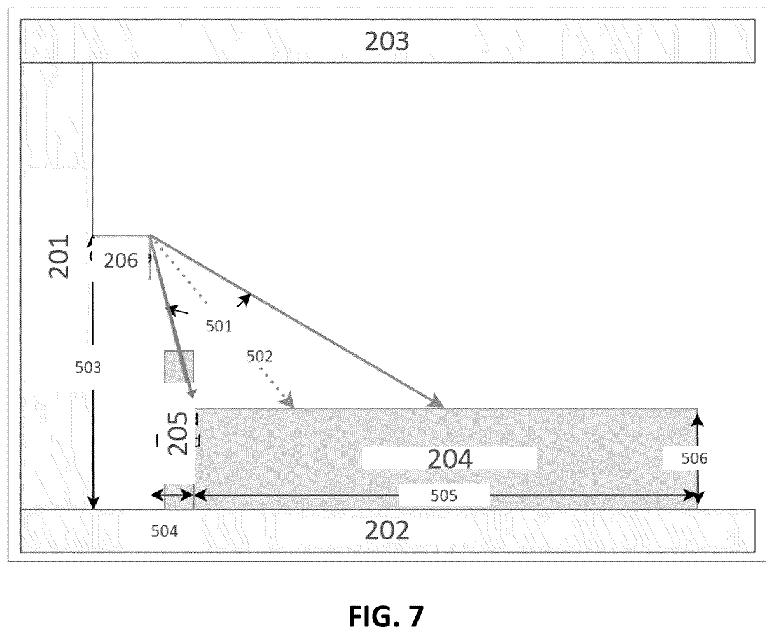

FIG. 7 illustrates a side view of a system represented as a schematic example system of the present disclosure. The system can comprise a capsule 206, mounted on a wall 201 above a headboard 205. The FIG. 5 illustrates an example of the system's geometry. The capsule comprises one or more sensors (e.g., a video camera, a thermal camera and/or an infrared camera). As depicted, an example field of view 501 for the one or more sensors is about 50 degrees for a video camera, and about 40 degrees for a thermal camera. An arbitrary center line 502 extends from the capsule to a bed 204. The example distance 503 from the top of the capsule to a floor 202 is about 1.42 m (56 in). The headboard 205 has an example width 504 of about 8 cm (3 in), while the bed 204 has an example length 505 of about 2.14 m (84 in) and an example height 506 of about 63.5 cm. The capsule 206 comprises a projector that generates a projection on a surface, such as a ceiling 203. The projection presents multifaceted information comprising one or more of time, temperature, environmental metrics, and sleep data.

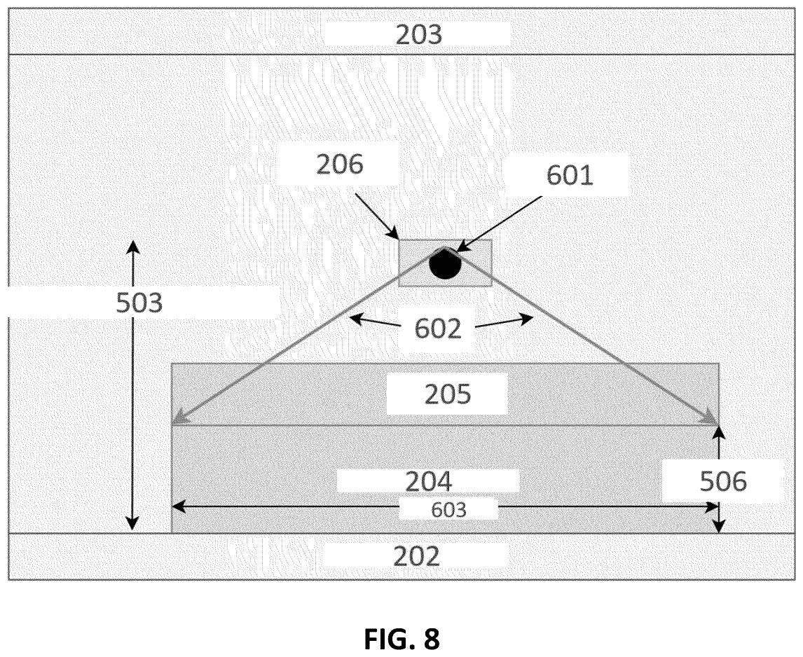

FIG. 8 illustrates a front view of a system represented as a schematic example system of the present disclosure. The system can comprise a capsule 206, mounted on a wall above a headboard 205 of a bed 204. The FIG. 6 illustrates an example of the system's geometry. The capsule comprises one or more sensors 601 (e.g., a video camera and/or a thermal camera). As depicted, an example field of view 602 for the one or more sensors is about 100 degrees for both a video camera and a thermal camera. An example distance 503 from the top of the capsule to a floor 202 is about 1.42 m (56 in). The bed 204 has an example width 603 of about 1.93 m (76 in). An example distance 506 from top of the bed 204 to the floor 202 is about 63.5 cm (25 in). The capsule 206 comprises a projector that generates a projection on a surface, such as a ceiling 203. The projection presents multifaceted information comprising one or more of time, temperature, environmental metrics, and sleep data.

### Example 6: Components of Capsule Projector

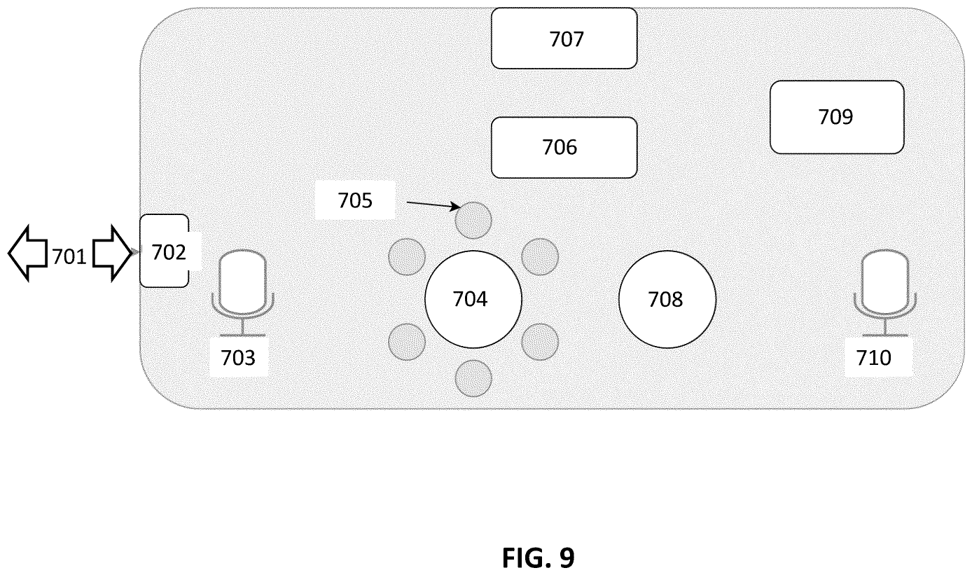

FIG. 9 illustrates an example schematic view of a capsule comprising one or more sensors. The capsule comprises a USB-C cable 701 and a USB-C port 702 used for transferring data obtained by the one or more sensors, a wire connection to a projector/display unit, and/or a power source. The capsule comprises one or more microphones on the left 703 and right 710 hand side of the capsule. The capsule comprises a night vision camera 704, which operates in tandem with an infrared illuminator 705. The capsule comprises a passive infrared (PIR) sensor 706 and a projector 707. The capsule comprises one or more sensors 709 used for monitoring environmental conditions (e.g., temperature, humidity or light).

### Example 7: Longitudinal Health Monitoring

Features of the systems and methods described herein provide users with a longitudinal assessment of their physiological health by continuously monitoring key biometrics such as breathing patterns, heartbeat patterns, and abnormal metric fluctuations. This process relies on passive, nocturnal monitoring via sensor-laden mattress pad, leveraging machine learning models to extract and analyze health trends over extended periods. Examples of output from the longitudinal health monitoring are shown in FIGS. 10A and 10B.

### Example 7A: Breathing Disturbance (Apnea) Detection

The breathing disturbance detection feature of the system is designed to monitor respiratory patterns, identify abnormalities, and detect potential apnea events in real time. This system integrates piezoelectric sensors, digital signal processing (DSP), and deep learning models to analyze mechanical vibrations caused by respiration and cardiac activity. By continuously processing physiological signals, the system enables non-invasive, long-term monitoring of sleep health.

There are four objectives of the breathing disturbance detection system: (1) detecting cessations in breathing, consisting of identifying apnea events characterized by prolonged pauses in respiration; (2) distinguishing true apnea from sensor displacement, consisting of ensuring that detected breathing interruptions are due to physiological events rather than movement artifacts; (3) providing real-time analysis with minimal latency, consisting of enabling rapid identification of disturbances and potential integration into health insights; and (4) optimizing computational efficiency, consisting of designing a system that can be deployed at scale for thousands of users in real-world conditions.

The system incorporates piezoelectric sensors embedded within its Active Grid mattress cover to passively and non-invasively monitor chest movements associated with breathing and cardiac activity. These sensors function by detecting mechanical deformations that generate electrical signals proportional to pressure changes. Sensors are strategically integrated into the mattress to capture movement at the chest level. Unlike wearable sensors, this setup ensures continuous and unobtrusive data collection.

The physiological signals captured include: respiratory effort (expansion and contraction of the chest due to breathing), ballistocardiography (BCG; subtle chest vibrations caused by cardiac activity), and snoring vibrations (low-amplitude signals caused by airway obstructions). There are multiple advantages of the piezoelectric sensing, including complete non-contact to reduce user burden, high sensitivity to detect even minor respiratory variation, and long-term monitoring to enable continuous sleep tracking over multiple nights.

The breathing disturbance detection has a two-stage processing pipeline. The first stage is digital signal processing (DSP). The first stage focuses on feature extraction and filtering to identify respiratory cycles and detect potential apnea events. This involves four steps.

First is preprocessing the raw piezoelectric signal. The raw signal, originally sampled at 500 Hz, is downsampled to 100 Hz to reduce computational complexity while preserving essential signal features. A bandpass filter is applied at 0.04 Hz to 0.5 Hz for respiratory detection, and 3 Hz to 21 Hz for cardiac activity. Signal artifacts from movement are mitigated using adaptive filtering techniques.

Second is implementation of the breath detection algorithm. Peak and trough identification identifies breath cycles using peak detection. Inter-Breath Interval (IBI) computation estimates respiration rate based on consecutive troughs. Breath variability assessment detects irregular patterns associated with disordered breathing.

Third is implementation of the heartbeat detection algorithm. Beat extraction isolates the cardiac component from the piezoelectric signal. Deep learning-based detection involves a pre-trained deep learning model that detects heartbeats and computes heart rate variability (HRV). Beat amplitude analysis ensures low-amplitude heartbeats are used to distinguish apnea from sensor displacement.

Fourth is cessation of breathing detection. Breath gap identification detects breath gaps exceeding 10 seconds per clinical apnea definitions. Intersection with low beat amplitude periods cross-references breath gaps with stable but low-amplitude heartbeats. Final apnea event classification flags apnea events if both conditions persist beyond the threshold.

The second stage of the breathing disturbance detection pipeline is Deep Learning-Based Refinement (ResNet Model). The model ingests a combination of raw waveforms, spectrograms, and frequency-domain representations to assist in feature extraction and input representation. Inputs are structured as (batch_size, 6000, 3), where 6000 corresponds to 60 seconds of data at 100 Hz and 3 represents: (1) Raw Piezoelectric Signal (chest motion); (2) Heart Rate Band-Passed Signal (3-21 Hz BCG component), or (3) Respiratory Band-Passed Signal (0.03-3 Hz breath component).

The ResNet architecture includes: network depth, three residual blocks optimized for physiological time-series data; convolutional layers which captures both short-and long-term signal dependencies; global average pooling, which reduces dimensionality while preserving key features; and output of a probability score indicating apnea likelihood. The model is trained on 18,630 unique samples, each annotated an average of 8.09 times, totaling 153,671 expert-labeled reads. Annotation Quality were labeled by Centaur Labs, using weighted consensus models and adaptive expertise weighting to ensure accuracy. The labels for these examples were created through human annotation with between 5 and 15 independent labels per example, ensuring robust and reliable annotation by multiple reviewers. Data Augmentation included time warping and noise injection to improve generalization.

Cloud-Based Inference: the model runs on AWS EC2 instances, allowing real-time processing for thousands of users. Data is streamed continuously via AWS Kinesis, ensuring minimal latency.

TensorFlow Lite Optimization: TFLite conversion reduces model size and speeds up inference. Optimized for CPU-based processing, balancing performance with cost.

Computational Sharding and Scalability: workload is distributed across multiple EC2 instances. The system ensures sub-minute processing time per user. Architecture scales automatically based on incoming data volume.

Real-Time Decision Pipeline: the model processes 1-minute overlapping windows for continuous monitoring. Final outputs are integrated into the mobile device application, providing user insights.

This structured approach ensures high sensitivity, scalability, and real-time performance for large-scale sleep monitoring and apnea detection.

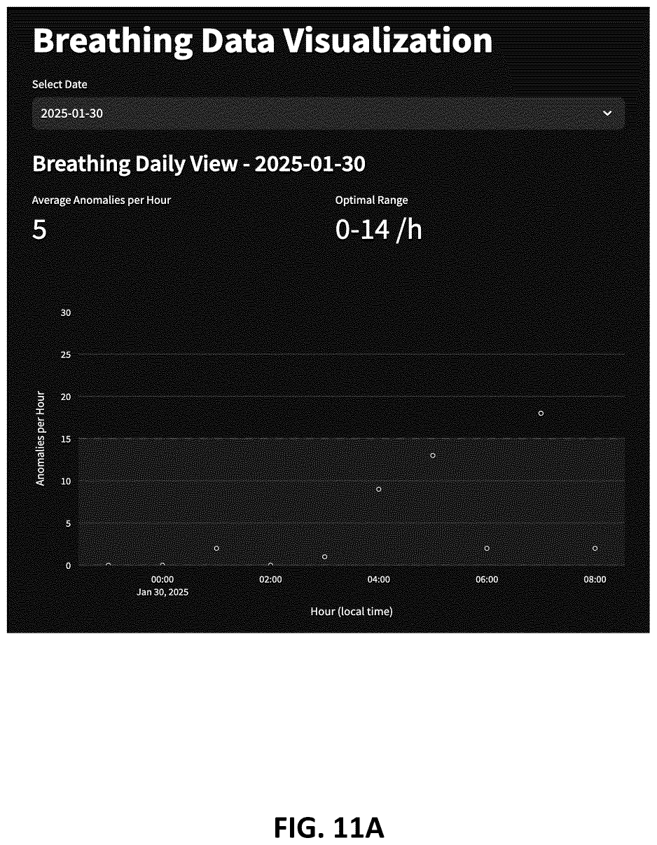

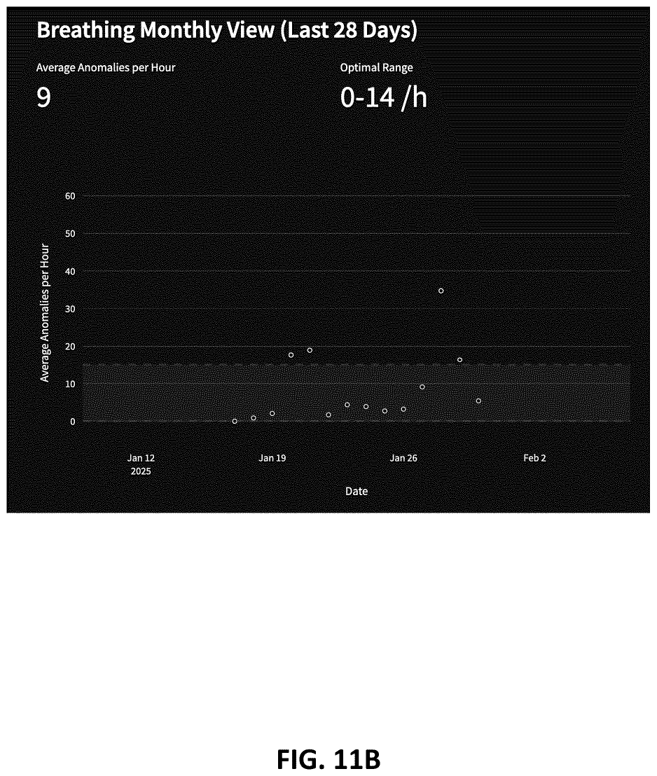

The initial DSP screening boasts a 66% positive predictive value at the minute level. The ResNet model further enhances accuracy, achieving an area under the ROC curve of 0.90. Examples of output from the breathing disturbance detection are shown in FIGS. 11A and 11B. FIG. 11A shows a daily view of the average anomalies per hour with a plot to show anomalies per hour over an 8-hour sleep session (i.e. hour 0:00 to 8:00). FIG. 11B shows a monthly view of the average anomalies per hour with a plot to show anomalies per hour over the previous 28 days.

### Example 7B: Arrhythmia Detection

The arrythmia detection feature of the system is designed to monitor cardiac patterns, identify abnormalities, and detect potential arrythmia events in real time. The sensors embedded in the system capture cardiovascular signals with each heartbeat. Regular heart rhythms exhibit minor variations in beat-to-beat intervals, known as heart rate variability (HRV). Arrhythmia disrupts this pattern, causing irregular heartbeat timing. By continuously processing physiological signals, the system enables non-invasive, long-term monitoring of sleep health.

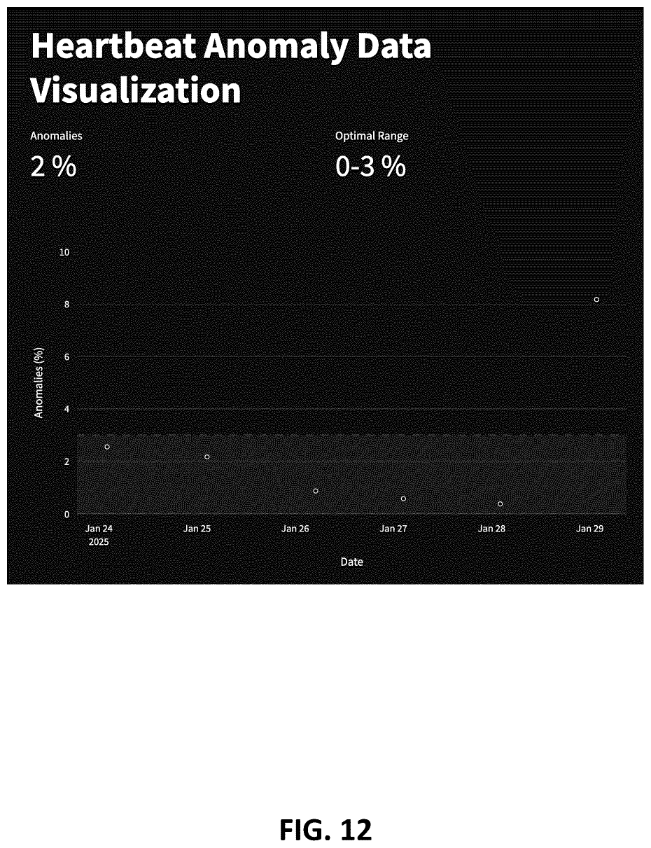

A multi-step process is employed for arrhythmia detection. First, a deep learning model identifies heartbeats from the sensor data. Then, a signal quality filtering process ensures that only high-confidence beats are included in the analysis. Finally, an irregular beat classification algorithm flags sections where the heart rate fluctuates significantly between consecutive beats, while actively filtering out normal respiratory sinus arrhythmia. The beat detection model was trained on over 80,000 minutes of heartbeat data from more than 70 participants. The system for analyzing the beats to detect arrhythmias was designed to prioritize precision over recall, aiming to minimize false positives. Evaluation of over 1,000 nights of flagged data, with manual review of a subset, confirmed the high accuracy of the arrhythmia detection algorithm. An example output of the arrythmia detection feature of the system is shown in FIG. 12. The visualization shows heartbeat anomaly data over six days.

Data was analyzed from over 1,000 random users using an applied irregular heartbeat detection algorithm. Setting the loading threshold at 3% resulted in an alert rate that aligned with the expected prevalence of irregular heartbeats in the general population. Other loading thresholds, including 2%, 4%, and 5% were considered, but 3% provided a balance between sensitivity and specificity.

The heartbeat detection algorithm is trained to reliably detect heartbeats from ballistocardiogram (BCG) signals collected by sensors in the system. A deep-learning U-Net model, trained on ECG-referenced clinical data, was used to identify beats accurately. To ensure high accuracy, two detection methods were combined: (1) a deep-learning-based beat detection, comprising the primary model to identify heartbeats with high precision; and (2) signal-processing-based detector, comprising a simpler method that provides a secondary verification of clean signals.

When both detectors agree, the detected beats were considered reliable. This method helps filter out noise-related artifacts. Once a clean set of beats was extracted, inter-beat intervals (IBIs) were computed. To improve accuracy, a time-series filter was applied at the respiration rate to remove respiratory sinus arrhythmia (RSA), a normal physiological phenomena, from the IBIs. This ensures that detected irregularities are more likely to be true pathological events rather than physiological variations. Normally, IBIs vary within ˜20% due to heart rate variability (HRV), and any beat deviating beyond this threshold is classified as an outlier. To calculate irregular heartbeat “loading,” the ratio of irregular beats to total beats was taken over a full night. Using a longer time span increases confidence in detecting meaningful trends while reducing sensitivity to short-term fluctuations.

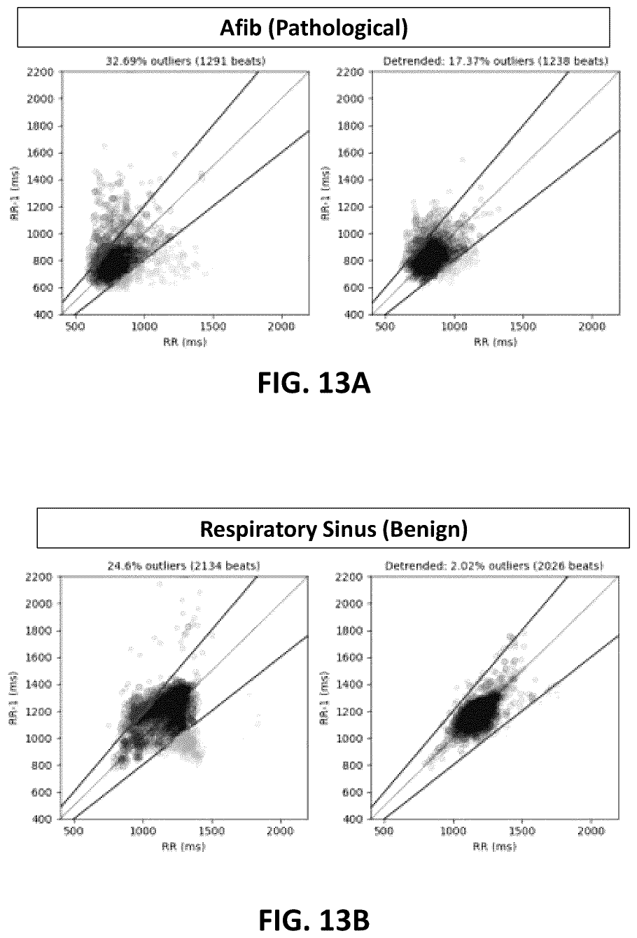

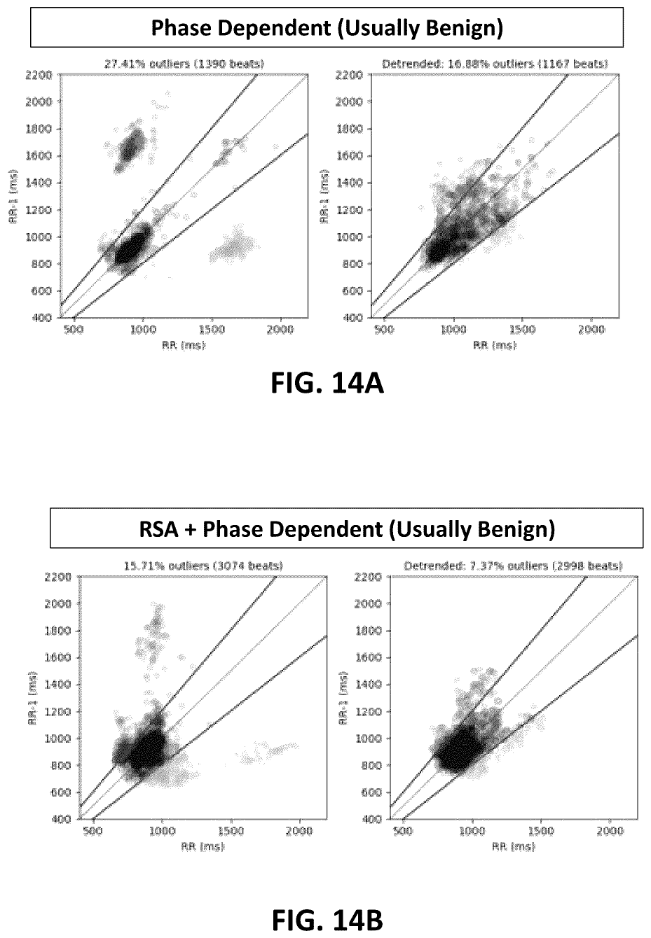

Poincaré plots were used to visualize the distribution of interbeat intervals (IBIs) and assess heartbeat irregularities. A Poincaré plot is a scatter plot where each beat interval (IBI_n) is plotted against the next interval (IBI_n+1). This helps differentiate between normal heart rate variability, benign irregularities, and pathological patterns such as atrial fibrillation (AFib). Example plots are shown in FIGS. 13A-13B and FIGS. 14A-14B. For each pair of plots, the left plot is before RSA removal, and the right plot is after filtering for RSA. FIG. 13A shows a plot for Afib patterns, comprising irregular and unstructured pattern, indicating chaotic electrical activity. Irregular beats are chaotic and scattered before RSA removal (left plot). After removal (right plot), the beats remain somewhat scattered and loading remains at over 17%. FIG. 13B shows a plot for RSA (benign) patterns, comprising distinct diagonal clustering due to breathing-induced heart rate variations.

Irregular beats are clumped just outside the cone before RSA removal (left plot). After RSA removal almost no irregularities remain (right plot). FIG. 14A shows phase-dependent irregularities, comprising irregularities tied to specific sleep or autonomic phases. Irregular beats are clumped in the corners of our plots before RSA removal (left plot). After removal, these clusters move closer to the cone (right plot). FIG. 14B shows RSA and phase-dependent irregularities, comprising combined features of RSA and phase-related variation. Both types of clusters exist before removal (left plot) and only the phase-dependent remain after (right plot).

### Example 7C: Wellbeing Monitoring

The wellbeing feature of the Health tier identifies trends and abnormalities in the cardio-respiratory health of users.

The wellbeing feature includes a two stage anomaly detection model. The first stage involves physiological biomarkers. The first stage extracts three physiological biomarkers from raw piezo data: (1) respiratory rate: first breath cycles are identified in the raw piezoelectric signal using peak detection, then respiration rate is estimated using the median time interval between consecutive troughs; (2) heart rate: a pre-trained deep learning model detects heartbeats in the raw piezo electric signal, and heart rate is estimated using the median time interval between consecutive heartbeats; and (3) heart rate variability (HRV): HRV is estimated using the variability of detect heart beats.

All three biomarkers are produced once per minute during sleep. First smoothing, filtering and bootstrapping are employed to identify one median value of each metric for the whole night of sleep.

The second stage, consisting of an exponential moving average (EMA) model, is a set of statistical models that estimates a personalized baseline for each user based on past data.

Each user's statistical model is a set of 21 EMA models (3 models (1 for each metric) for each day of the week). The different models for each day of the week allow the models to capture the weekly behavioral trend that occurs due to weekday and weekend and separate the variable behavior from the underlying physiological health of the user. Each EMA model contains 3 numbers: mean, variance and history. The mean and standard deviation are the estimated values for the metric/day of week and history is the number of days used to make this estimate.

The model was not trained like a deep learning model. Data analysis was performed on 1.5 million nights to understand the patterns of heart and respiratory rates. Over 600 tags were used that were provided by users through the mobile phone app that indicated nights with labels such as sickness, late meal, alcohol intake, and exercise to understand the common patterns of anomalies. Based on these measures, the day of the week EMA model structure was designed to most closely approximate the distribution of physiological metrics for each user.

On day 0, the mean and std of each metric are initialized to the average of the whole population, and history is initialized to 0. Thus, the defaults are identical across the day of the week, but different for the 3 metrics. Each night 3 metric values for the three physiological biomarkers were measured (i.e., respiratory rate, heart rate, and HRV). Using these values, the EMA model mean and variance were updated. The ‘exponential’ refers to the weighting scheme, wherein new values are weighed exponentially with respect to the historical value. When history is 0, the new value has equal weight as the default value, and when history is large, the new values are weighed less than the current value. Therefore with growing history, the EMA converges to a stable mean and variance value.

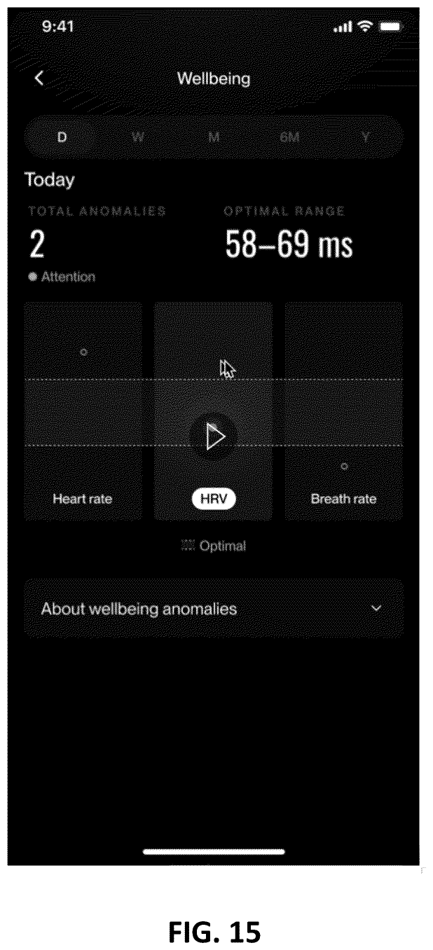

When a new value is detected which deviates strongly from the expected EMA value, for example, as measured by new value>2 standard deviations away from expected value, the value is flagged as an anomalous value. Each day the number of anomalies detected was shared (minimum 0, maximum 3 if all 3 metrics are out of expected range). As shown in the example output schematic of FIG. 15, the total number of anomalies for that day was 2.

The efficacy of this anomaly detection schema was tested in detecting health events. The schema was tested on a small number of users over 3 months. Single anomalies (i.e., anomalies of 1 out of 3 metrics) occurred for many benign reasons such as a late meal. Double anomalies (i.e., anomalies of 2 out of 3 metrics) usually were more reliably a result by some cause, however, the reasons were often still benign such as a late meal or late sleep session. However, multiple nights of two or more anomalies usually indicated a health event such as jetlag or many nights of late meals/alcohol consumption. Triple anomalies (all 3 metrics) were rare and almost always related to a health event. We surveyed 15 users who triggered triple anomalies and determined the following breakdown: eight users had sickness (cold/flu/fever), two users had alcohol consumption and/or late nights, two users had insomnia, two users had vigorous late night exercise, and 1 user had a flu shot. The key indicator of sickness that was not shared by other symptoms was that sickness usually lasted 2-5 days, with a mix of double and triple anomalies while all the others only happened on isolated nights.

Due to the breadth of possible events that could be detected, the accuracy assessment was based on recall or false positive rate, i.e. if an anomaly is flagged, does the user report an outlier event that affects their cardiorespiratory health? Out of 1500 user nights of testing, 147 people responded to our survey and received a double or triple anomaly. Of these, only 1 reported that the outlier was incorrect and another 2 reported that they did not have any sickness but there could have been stress affecting their metrics. Therefore the expected user perception false positive rate are approximately 1-2%.

### Example 7D: Health Tier Study

A Health Tier Study was conducted with 90 participants to gather real-world data on these health metrics and user experiences. The study aimed to recruit 30 normal individuals, 30 with arrhythmia, and 30 with sleep apnea. Participants were provided with daily feedback via a dashboard and asked to complete surveys about their experience.

Based on the results of the study we found that users perceived the algorithms to be highly accurate. Breathing perceived accuracy was 95.9% (803 out of 837 reported accurate data). Heartbeat perceived accuracy was 97.1% (799 out of 823 reported accurate data). Wellbeing perceived accuracy was 95.6% (769 out of 804 reported accurate data).

### EMBODIMENTS

The following non-limiting embodiments provide illustrative examples of the invention, but do not limit the scope of the invention.
Embodiment 1. A system for monitoring a physiological feature of a subject, comprising: a sensing device comprising a housing and at least one sensor in optical communication with an outer environment of the housing, wherein the at least one sensor is configured to detect a user electromagnetic radiation signal that is reflected or emitted by at least a portion of a body of the subject; a reference device comprising at least one optical source configured to output a reference electromagnetic radiation signal; and a processor operatively coupled to the sensing device and the reference device, configured to: (a) direct the reference device to output the reference electromagnetic radiation signal; (b) direct the sensing device to detect the reference electromagnetic radiation signal and the user electromagnetic radiation signal; and (c) determine the physiological feature of the subject based on both of the reference electromagnetic radiation signal and the user electromagnetic radiation signal,
optionally wherein:
(1) the physiological feature of the subject is determined based on a difference or similarity in wavelengths between the reference electromagnetic radiation signal and the user electromagnetic radiation signal; and/or
(2) the sensing device and the reference device are parts of a same device; and/or
(3) the sensing device and the reference are disposed relative to each other such that the reference electromagnetic radiation signal is outputted to an optical field of view of the at least one sensor; and/or
(4) the sensing device and the reference device are movable from one another; and/or
(5) the processor is configured to generate an image or video based on the user electromagnetic radiation signal; and/or
(6) the processor is configured to generate an image or video based on the user electromagnetic radiation signal and the reference electromagnetic radiation signal; and/or
(7) the image or video is a whole-body image or video of the subject; and/or
(8) the processor is configured to change a wavelength or wavelength range of the reference electromagnetic radiation signal; and/or
(9) the change is based on the user electromagnetic radiation signal detected by the at least one sensor; and/or
(10) the reference electromagnetic radiation signal comprises a wavelength that is similar to that of an expected user electromagnetic radiation signal; and/or
(11) the at least one optical source is configured to output an additional reference electromagnetic radiation signal, wherein the reference electromagnetic radiation signal and the additional reference electromagnetic radiation signal comprise different wavelengths or wavelength ranges; and/or
(12) the processor is configured to determine a relative distance between the at least one sensor and the user based on the user electromagnetic radiation signal; and/or
(13) the processor is configured to identify a size of the at least the portion of the body of the subject based on the user electromagnetic radiation signal, wherein the relative distance is determined based on the size; and/or
(14) the processor is configured to (i) retrieve historical data of the user electromagnetic radiation signal of the user from the past and (ii) determine the physiological feature based on a comparison between a new detection of the user electromagnetic radiation signal and the historical data; and/or
(15) the processor is configured to (i) determine a biological signal of the user based on analyzing the reference electromagnetic radiation signal and the user electromagnetic radiation signal and (ii) determine the physiological feature based on the biological signal; and/or
(16) the system further comprises an additional sensing device comprising at least one contact sensor configured to detect a contact sensor signal when in direct or indirect contact with the user, wherein the processor is further configured to (i) direct the at least one contact sensor to detect the contact sensor signal, and (ii) determine the physiological feature of the subject based on the contact sensor signal and the user electromagnetic radiation signal; and/or
(17) the additional sensing device is part of an article of furniture configured to be in contact with the user; and/or
(18) the at least one sensor comprises an infrared sensor, and wherein the user electromagnetic radiation signal comprises an infrared wavelength ranging between about 1 micrometer to about 15 micrometer; and/or
(19) the reference electromagnetic radiation signal comprises an infrared wavelength ranging between about 1 micrometer to about 15 micrometer; and/or
(20) the at least one sensor further comprises a visible-light sensor configured to detect a visible light signal associated with the user or the user's environment, wherein the processor is configured to align the visible light signal and the user electromagnetic radiation signal to generate a multi-modal imaging data usable for determining the physiological feature; and/or
(21) the physiological feature is a health condition or disease of the subject; and/or
(22) the physiological feature is a gesture of the subject in the dark, wherein the controller is configured to operate one or more user devices based on the gesture of the subject; and/or
(23) a relative position between the housing and the at least one sensor of the sensing device is adjustable; and/or
(24) the system is part of a fixture configured to be in proximity to or adjacent to an article of furniture; and/or
(25) the system further comprises the article of furniture; and/or
(26) the processor is configured to modify a condition of the article of furniture based on the determined physiological feature of the subject.
Embodiment 2. A method for monitoring a physiological feature of a subject, comprising: (a) outputting, using a reference device comprising an optical source, to output a reference electromagnetic radiation signal; (b) detecting, using a sensing device comprising a housing and at least one sensor in optical communication with an outer environment of the housing, (i) the reference electromagnetic radiation signal and (ii) a user electromagnetic radiation signal that is reflected or emitted by at least a portion of a body of the subject; and (c) determining the physiological feature of the subject based on both of the reference electromagnetic radiation signal and the user electromagnetic radiation signal,
optionally wherein:
(1) the physiological feature of the subject is determined based on a difference or similarity in wavelengths between the reference electromagnetic radiation signal and the user electromagnetic radiation signal; and/or
(2) the sensing device and the reference device are parts of a same device; and/or
(3) the sensing device and the reference are disposed relative to each other such that the reference electromagnetic radiation signal is outputted to an optical field of view of the at least one sensor; and/or
(4) the sensing device and the reference device are movable from one another; and/or
(5) the method further comprises generating an image or video based on the user electromagnetic radiation signal; and/or
(6) the method further comprises generating an image or video based on the user electromagnetic radiation signal and the reference electromagnetic radiation signal; and/or
(7) the image or video is a whole-body image or video of the subject; and/or
(8) the method further comprises changing a wavelength or wavelength range of the reference electromagnetic radiation signal; and/or
(9) the changing is based on the user electromagnetic radiation signal detected by the at least one sensor; and/or
(10) the reference electromagnetic radiation signal comprises a wavelength that is similar to that of an expected user electromagnetic radiation signal; and/or
(11) the method further comprises directing the at least one optical source to output an additional reference electromagnetic radiation signal, wherein the reference electromagnetic radiation signal and the additional reference electromagnetic radiation signal comprise different wavelengths or wavelength ranges; and/or
(12) the method further comprises determining a relative distance between the at least one sensor and the user based on the user electromagnetic radiation signal; and/or
(13) the method further comprises identifying a size of the at least the portion of the body of the subject based on the user electromagnetic radiation signal, wherein the relative distance is determined based on the size; and/or
(14) the method further comprises (i) retrieving historical data of the user electromagnetic radiation signal of the user from the past and (ii) determining the physiological feature based on a comparison between a new detection of the user electromagnetic radiation signal and the historical data; and/or
(15) the method further comprises (i) determining a biological signal of the user based on analyzing the reference electromagnetic radiation signal and the user electromagnetic radiation signal and (ii) determining the physiological feature based on the biological signal; and/or
(16) the method further comprises using an additional sensing device comprising at least one contact sensor configured to detect a contact sensor signal when in direct or indirect contact with the user, (i) directing the at least one contact sensor to detect the contact sensor signal, and (ii) determining the physiological feature of the subject based on the contact sensor signal and the user electromagnetic radiation signal; and/or
(17) the additional sensing device is part of an article of furniture configured to be in contact with the user; and/or
(18) the at least one sensor comprises an infrared sensor, and wherein the user electromagnetic radiation signal comprises an infrared wavelength ranging between about 1 micrometer to about 15 micrometer; and/or
(19) the reference electromagnetic radiation signal comprises an infrared wavelength ranging between about 1 micrometer to about 15 micrometer; and/or
(20) the at least one sensor further comprises a visible-light sensor configured to detect a visible light signal associated with the user or the user's environment, wherein the method further comprises aligning the visible light signal and the user electromagnetic radiation signal to generate a multi-modal imaging data usable for determining the physiological feature; and/or
(21) the physiological feature is a health condition or disease of the subject; and/or
(22) the physiological feature is a gesture of the subject in the dark, wherein the method further comprises operating one or more user devices based on the gesture of the subject; and/or
(23) a relative position between the housing and the at least one sensor of the sensing device is adjustable; and/or
(24) the sensing device is part of a fixture that is in proximity to or adjacent to an article of furniture; and/or
(25) the method further comprises modifying a condition of the article of furniture based on the determined physiological feature of the subject.
Embodiment 3. A system for monitoring a physiological feature of a subject, comprising: a sensing device comprising a housing and at least one sensor in sensing communication with an outer environment of the housing, wherein the at least one sensor is configured to detect a user signal that is reflected or emitted by at least a portion of a body of the subject; a reference device comprising at least one signal source configured to output a reference signal; and a processor operatively coupled to the sensing device and the reference device, configured to: (a) direct the reference device to output the reference signal; (b) direct the sensing device to detect the reference signal and the user radiation signal; and (c) determine a physiological feature of the subject based on both of the reference signal and the user signal.
Embodiment 4. A method for monitoring a physiological feature of a subject, comprising: (a) outputting, using a reference device comprising a signal source, to output a reference signal; (b) detecting, using a sensing device comprising a housing and at least one sensor in sensing communication with an outer environment of the housing, (i) the reference signal and (ii) a user signal that is reflected or emitted by at least a portion of a body of the subject; and (c) determining the physiological feature of the subject based on both of the reference signal and the user signal.
Embodiment 5. A system for monitoring a physiological feature of a subject, comprising: an article of furniture comprising a contact sensor configured to detect a contact sensor signal associated with the subject when the subject is disposed over a top surface of the article of furniture; a sensing device comprising an optical sensor in optical communication with the top surface of the article of furniture, wherein the optical sensor is configured to detect a user electromagnetic radiation signal that is reflected or emitted by at least a portion of a body of the subject; and a processor operatively coupled to the contact sensor and the optical sensor, configured to: (a) direct the contact sensor to detect the contact sensor signal; (b) direct the optical sensor to detect the user electromagnetic radiation signal; (c) determine the physiological feature of the subject based on both of the contact sensor signal and the user electromagnetic radiation signal,
optionally wherein:
(1) both of the contact sensor signal and the user electromagnetic radiation signal are associated with or indicative of a same biological signal type of the subject; and/or
(2) the same biological signal type is a heart signal, a breathing signal, a perspiration signal, a motion signal, or a neurological signal; and/or
(3) the processor is configured to generate a new sensing data based on the contact sensor signal and the user electromagnetic radiation signal, wherein the new sensing data is usable to determine the physiological feature; and/or
(4) the processor is configured to (i) deter-mine a first heart signal data associated with plethysmograph (PG) signal of the user based on the user electromagnetic radiation signal, (ii) determine a second heart signal data associated with electrocardiograph (ECG) signal of the user based on the contact sensor signal, and (iii) deter-mine a pulse transit time (PTT) of the user based on the first heart signal data and the second heart signal data, wherein the PPT is usable for determining the physiological feature; and/or
(5) the processor is configured to determine aortic pulse wave velocity (APWV) based on the PTT, wherein the APWV is usable for determining the physiological feature; and/or
(6) the physiological feature is arterial stiffness; and/or
(7) the contact sensor is not a wearable sensor; and/or
(8) the contact sensor comprises a pressure sensor; and/or
(9) the pressure sensor comprises a piezoelectric sensor; and/or
(10) the optical sensor comprises an infrared sensor, and wherein the user electromagnetic radiation signal comprises an infrared wavelength ranging between about 1 micrometer to about 15 micrometer; and/or
(11) the article of furniture is a bed device; and/or
(12) the bed device is a mattress or a cover thereof; and/or
(13) the article of furniture and the sensing device are movable relative to one another; and/or
(14) a vertical position of the optical sensor is higher than a vertical position of the contact sensor; and/or
(15) the physiological feature is a health condition or disease of the subject.
Embodiment 6. A method for monitoring a physiological feature of a subject, comprising: (a) detecting, using a contact sensor of an article of furniture, a contact sensor signal associated with the subject when the subject is disposed over a top surface of the article of furniture; (b) detecting, using a sensing device comprising an optical sensor in optical communication with the top surface of the article of furniture, a user electromagnetic radiation signal that is reflected or emitted by at least a portion of a body of the subject; and (c) determining the physiological feature of the subject based on both of the contact sensor signal and the user electromagnetic radiation signal,
optionally wherein:
(1) both of the contact sensor signal and the user electromagnetic radiation signal are associated with or indicative of a same biological signal type of the subject; and/or
(2) the same biological signal type is a heart signal, a breathing signal, a perspiration signal, a motion signal, or a neurological signal; and/or
(3) the method further comprises generating a new sensing data based on the contact sensor signal and the user electromagnetic radiation signal, wherein the new sensing data is usable to determine the physiological feature; and/or
(4) the method further comprises (i) determining a first heart signal data associated with plethysmograph (PG) signal of the user based on the user electromagnetic radiation signal, (ii) determining a second heart signal data associated with electrocardiograph (ECG) signal of the user based on the contact sensor signal, and (iii) determining a pulse transit time (PTT) of the user based on the first heart signal data and the second heart signal data, wherein the PPT is usable for determining the physiological feature; and/or
(5) the method further comprises determining aortic pulse wave velocity (APWV) based on the PTT, wherein the APWV is usable for determining the physiological feature; and/or
(6) the physiological feature is arterial stiffness; and/or
(7) the contact sensor is not a wearable sensor; and/or
(8) the contact sensor comprises a pressure sensor; and/or
(9) the pressure sensor comprises a piezoelectric sensor; and/or
(10) the optical sensor comprises an infrared sensor, and wherein the user electromagnetic radiation signal comprises an infrared wavelength ranging between about 1 micrometer to about 15 micrometer; and/or
(11) the article of furniture is a bed device; and/or
(12) the bed device is a mattress or a cover thereof; and/or
(13) the article of furniture and the sensing device are movable relative to one another; and/or
(14) a vertical position of the optical sensor is higher than a vertical position of the contact sensor; and/or
(15) the physiological feature is a health condition or disease of the subject.
Embodiment 7. A system for enhancing sleep of a user, the system comprising: a sleep enhancement device comprising a regulator and a bed device having an at least partially internal heating or cooling element, wherein the regulator is configured to adjust at least one sleep-related parameter for the bed device to enhance sleep of the user; and a projector configured to project an optical pattern to a wall or a ceiling adjacent the bed device, wherein the optical pattern provides at least one of (a) status information for the bed device, local environment, or both or (b) at least one control option to direct operation of the sleep enhancement device,
optionally wherein:
(1) the bed device comprises a mattress comprising the at least partially internal cooling or heating element; and/or
(2) the bed device comprises a mattress cover comprising the at least partially internal cooling or heating element; and/or
(3) the sleep enhancement device comprises a console comprising a temperature regulator; and/or
(4) the temperature regulator is operatively coupled to the at least partially internal cooling or heating element of the bed device; and/or
(5) the at least partially internal heating or cooling element comprises one or more fluid channels positioned at least partially within a body of the bed device; and/or
(6) the at least one sleep-related parameter that is adjusted by the regulator comprises at least one temperature of the bed device, the user, or both; and/or
(7) the system further comprises a sensor (e.g., a sensing device comprising an optical device or a non-contact sensor as provided herein) coupled to the bed device, wherein the sensor is configured to detect a parameter of the bed device, the user, or both; and/or
(8) the sensor is operatively coupled to the regulator, and wherein the regulator is configured to adjust the at least one sleep-related parameter for the bed device based on the detected parameter; and/or
(9) the sensor comprises an infrared sensor; and/or
(10) the system further comprises an infrared light source operatively coupled to the infrared sensor, wherein the infrared light source is configured to direct infrared light towards the bed device, the user, or both; and/or
(11) the system further comprises a housing containing at least a portion of the projector and at least a portion of the sensor; and/or
(12) the housing is in the form of a headboard for a mattress; and/or
(13) the housing is in the form of a nightstand; and/or
(14) the housing is in the form of a wall furniture; and/or
(15) the sensor is configured to detect the parameter of the user, wherein the parameter of the user comprises one or more of a temperature of the user, a humidity of the user, a movement level of the user, a sleep phase of the user, a body posture of the user, a facial skin mole of the user, an arterial stiffness of the user, a fever of the user, a menstrual cycle of the user, or a bodily injury of the user; and/or
(16) the sensor is configured to detect the parameter of the bed device, wherein the parameter of the bed device comprises a temperature of the bed device, a temperature of an environment adjacent the bed device, a humidity of the bed device, or a humidity of an environment adjacent the bed device; and/or
(17) the regulator is configured to adjust the at least one sleep-related parameter based on a presence of the user on the bed device; and/or
(18) the bed device is adjusted for bed device position, bed device orientation, or both; and/or
(19) the sleep enhancement device comprises an adjustable mattress frame configured to be coupled to a mattress and to adjust a position, an orientation, or both of at least a portion of the mattress; and/or
(20) the status information comprises one or more of the present time, a temperature of the user, a temperature of the bed device, a temperature of an environment adjacent the bed device, a humidity of the user, a humidity of the bed device, a humidity of an environment adjacent the bed device, a movement level of the user, a sleep phase of the user, a body posture of the user, an arterial stiffness of the user, a fever of the user, a menstrual cycle status of the user, a bodily injury of the user, or an energy level of the sleep enhancement device; and/or
(21) the at least one control option is selectable to adjust one or more of temperature of the bed device, bed device position, bed device orientation, a wake-up time setting of the sleep enhancement system, a time and date setting of the sleep enhancement device, a user or user profile information for the sleep enhancement device, or a location information for the sleep enhancement device; and/or
(22) the system further comprises (i) a motion detector operatively coupled to the processor, wherein the motion detector is configured to detect at least one gesture of the user, and (ii) a processor and computer memory coupled to the processor, wherein the computer memory comprises instructions for the processor to select the at least one control option based on the detected at least one gesture; and/or
(23) the optical pattern comprises a graphical user interface (GUI) for the user to select the at least one control option; and/or
(24) the system further comprises a cooling fan operatively coupled to the projector, wherein the cooling fan is configured to be turned on upon operation of the projector for more than a predetermined threshold duration of time.
Embodiment 8. A computer-implemented method for enhancing sleep of a user, the method comprising: adjusting at least one sleep-related parameter for a bed device of a sleep enhancement device, thereby enhancing sleep of a user of the bed device, wherein the bed device comprises an at least partially internal heating or cooling element; and projecting, via a projector, an optical pattern to a wall or a ceiling adjacent the bed device, wherein the optical pattern provides at least one of (a) status information for the bed device, local environment, or both or (b) at least one control option to direct operation of the sleep enhancement device,
optionally wherein:
(1) the adjusted at least one sleep-related parameter comprises at least one temperature of the bed device, the user, or both; and/or
(2) the method further comprises detecting, via a sensor, a parameter of the bed device, the user, or both; and/or
(3) the method further comprises adjusting the at least one sleep-related parameter for the bed device based on the detected parameter; and/or
(4) the sensor comprises an infrared sensor; and/or
(5) the method further comprises directing, via an infrared light source, infrared light towards the bed device, the user, or both; and/or
(6) the method further comprises detecting the parameter of the user, wherein the parameter of the user comprises one or more of a temperature of the user, a humidity of the user, a movement level of the user, a sleep phase of the user, a body posture of the user, a facial skin mole of the user, an arterial stiffness of the user, a fever of the user, a menstrual cycle of the user, or a bodily injury of the user; and/or
(7) the method further comprises detecting the parameter of the bed device, wherein the parameter of the bed device comprises a temperature of the bed device, a temperature of an environment adjacent the bed device, a humidity of the bed device, or a humidity of an environment adjacent the bed device; and/or
(8) the method further comprises detecting a presence of the user on the bed device and adjusting the at least one sleep-related parameter based on the detected presence; and/or
(9) the at last one sleep-related parameter comprises bed device position, bed device orientation, or both; and/or
(10) the method further comprises controlling an adjustable mattress frame coupled to a mattress, to adjust a position, an orientation, or both of at least a portion of the mattress; and/or
(11) the status information comprises one or more of the present time, a temperature of the user, a temperature of the bed device, a temperature of an environment adjacent the bed device, a humidity of the user, a humidity of the bed device, a humidity of an environment adjacent the bed device, a movement level of the user, a sleep phase of the user, a body posture of the user, an arterial stiffness of the user, a fever of the user, a menstrual cycle status of the user, a bodily injury of the user, or an energy level of the sleep enhancement device; and/or
(12) the at least one control option is selectable to adjust one or more of temperature of the bed device, bed device position, bed device orientation, a wake-up time setting of the sleep enhancement system, a time and date setting of the sleep enhancement device, a user or user profile information for the sleep enhancement device, or a location information for the sleep enhancement device; and/or
(13) the method further comprises (i) detecting, via a motion detector, at least one gesture of the user and (ii) selecting the at least one control option from a computer memory based on the detected at least one gesture; and/or
(14) the optical pattern comprises a graphical user interface (GUI) for the user to select the at least one control option.
Embodiment 9. A computer-implemented method for detecting a condition of a user of an article of furniture, the method comprising: determining, by a first computer algorithm, one or more sensor data signatures indicative of a physiological condition of the user via based on an input sensor data associated with a biological signal of the user, wherein the input sensor data is collected by a biological sensor while the user is sleeping on the article of furniture; and confirming, by a second and different computer algorithm, the determined one or more sensor data signatures based on (i) at least a portion of the input sensor data and (ii) the determined one or more sensor data signatures,
optionally wherein:
(1) the method further comprises providing (i) the at least the portion of the input sensor data and (ii) the determined one or more sensor data signatures as inputs for the second and different computer algorithm; and/or
(2) the first computer algorithm comprises digital signal processing (DSP); and/or
(3) the second and different computer algorithm comprises a residual neural network (ResNet) model; and/or
(4) the second and different computer algorithm comprises a convolution neural network; and/or
(5) the confirming comprises calculating an accuracy of the determined one or more sensor data signatures; and/or
(6) the method further comprises determining the physiological condition of the user by the second and different computer algorithm; and/or
(7) the one or more data signatures comprise respiratory rate, breath cycle, heart rate, heart rate variability, or vibration; and/or
(8) the one or more data signatures comprise snoring vibration; and/or
(9) the one or more data signatures are produced at least once per minute while the user is sleeping on the article of furniture; and/or
(10) the one or more data signatures are produced at most once per minute while the user is sleeping on the article of furniture; and/or
(11) the biological sensor comprises a piezoelectric sensor, a temperature sensor, a ballistocardiograph, or any combination thereof; and/or
(12) the biological sensor comprises a piezoelectric sensor; and/or
(13) the first computer algorithm is configured to downsample the input sensor data collected by the biological sensor; and/or
(14) the determining further comprises filtering the one or more sensor data signatures indicative of a physiological condition of the user using the first computer algorithm; and/or
(15) the first computer algorithm comprises a breath detection algorithm and/or a heartbeat detection algorithm; and/or
(16) the breath detection algorithm is configured to identify one or more breath cycles, estimate respiration rate of the user, detect irregular breathing patterns, or any combination thereof; and/or
(17) the heartbeat detection algorithm is configured to isolate cardiac activity from the input sensor data; and/or
(18) the heartbeat detection algorithm is configured to detect one or more heartbeats and/or calculate a heart rate variability (HRV); and/or
(19) the first computer algorithm is configured to detect a breath gap; and/or
(20) a breath gap is a cessation in breathing lasting at least 5 seconds; and/or
(21) a breath gap is a cessation in breathing lasting at least 10 seconds; and/or
(22) the first computer algorithm is configured to cross-reference a first data type extracted from the input sensor data with a second data type extracted from the input sensor data; and/or
(23) the first data type is a breath gap and the second data type is a heartbeat; and/or
(24) the article of furniture is a bed device; and/or
(25) the bed device is a mattress; and/or
(26) the bed device is a mattress cover; and/or
(27) the determining and/or the confirming occur in real-time; and/or
(28) the method further comprises training the second and different computer algorithm; and/or
(29) the training comprises human labeling; and/or
(30) the condition of the user is a cardio-respiratory condition; and/or
(31) the condition of the user is apnea, arrhythmia, flu, cold, fever, or any combination thereof; and/or
(32) the condition of the user is a physiological stressor; and/or
(33) an accuracy of detecting the condition of the user is improved with the first computer algorithm and the second and different computer algorithm as compared to an accuracy of detecting the condition of the user with the first computer algorithm alone; and/or
(34) the second and different computer algorithm comprises one or more Exponential Moving Average (EMA) models; and/or
(35) the one or more Exponential Moving Average (EMA) models are configured to calculate a metric value for each of the one or more sensor data signatures; and/or
(36) the method further comprises comparing the metric value with a historical value of each of the one or more sensor data signatures; and/or
(37) the method further comprises generating a notification if the metric value is different from the historical value; and/or
(38) the notification is generated if the metric value is greater than 2 standard deviations away from the historical value; and/or
(39) the method further comprises providing a report of the notification to the user via a graphical user interface (GUI).
Embodiment 10. A system comprising: a computer processor and a computer memory coupled thereto, wherein the computer memory comprises a machine executable code that, upon execution by the one or more computer processors, implements the method of embodiment 9.
Embodiment 11. A computer-implemented method for detecting a condition of a user of an article of furniture, the method comprising: (a) determining, by a first computer algorithm, one or more sensor data signatures indicative of a physiological condition of the user via based on an input sensor data associated with a biological signal of the user, wherein the input sensor data is collected by a biological sensor while the user is sleeping on the article of furniture; (b) classifying, by a second and different computer algorithm, the determined one or more sensor data signatures of (a) as normal one or more sensor data signatures or irregular one or more sensor data signatures; and (c) filtering the irregular one or more sensor data signatures,
optionally wherein:
(1) the first computer algorithm comprises a convolutional neural network and/or;
(2) wherein the convolutional neural network is a U-Net model and/or;
(3) the U-Net model is trained on clinical data and/or;
(4) the clinical data is electrocardiogram data and/or;
(5) the one or more data signatures comprise respiratory rate, breath cycle, heart rate, heart rate variability, or vibration and/or;
(6) the one or more data signatures comprise heart rate and/or;
(7) further comprising verifying the determined one or more sensor data signatures of (a) prior to the classifying of (b) and/or;
(8) the verifying is by a signal-processing-based detector and/or;
(9) the biological signal of the user is a ballistocardiogram (BCG) signal and/or;
(10) the biological sensor comprises a piezoelectric sensor, a temperature sensor, a ballistocardiograph, or any combination thereof and/or;
(11) the method further comprises computing one or more interbeat intervals (IBIs) based on the determined one or more sensor data signatures and/or;
(12) the filtering comprises applying a time-series filter at the respiration rate to remove respiratory sinus arrhythmia (RSA) and/or;
(13) the condition of the user is arrhythmia and/or;
(14) the method further comprises computing a loading rate, wherein the loading rate comprises a ratio of a number of the irregular one or more sensor data signatures to a number of the determined one or more sensor data signatures and/or;
(15) the method further comprises generating a notification if the loading rate is from 1% to 5% and/or;
(16) the method further comprises generating a notification if the loading rate is at least 3%.
Embodiment 12. A system comprising: a computer processor and a computer memory coupled thereto, wherein the computer memory comprises a machine executable code that, upon execution by the one or more computer processors, implements the method of embodiment 11.
Embodiment 13. A computer-implemented method for detecting an anomaly in a physiological condition of a user of an article of furniture, the method comprising: (a) predicting a future sensor data or analysis data thereof (future data) based on a historical sensor data set associated with the user of an article of furniture, wherein the future data is indicative of a physiological condition of the user; and (b) determining a difference between the predicted future data and a new sensor data or analysis data thereof (new data), wherein the difference is indicative of the anomaly in the physiological condition of the user,
optionally wherein:
(1) the article of furniture is a mattress, a blanket, a pillow, or a cover thereof and/or;
(2) the historical sensor data set is collected by a user sensor while the user is sleeping on the article of furniture and/or;
(3) the user sensor is a part of the article of furniture and/or;
(4) the historical sensor data set comprises historical sensor data collected over a plurality of past uses of the article of furniture and/or;
(5) the plurality of past uses comprises a plurality of past consecutive uses and/or;
(6) the method further comprises identifying from the historical sensor data set one or more sensor data signatures associated with the physiological condition of the user, wherein the predicting the future data is based on the one or more sensor data signatures and/or;
(7) the one or more sensor data signatures comprise a range of sensor data values and/or;
(8) the one or more sensor data signatures comprise a pattern of sensor data and/or;
(9) the one or more sensor data signatures comprise respiratory rate, breath cycle, heart rate, heart rate variability, vibration, or any combination thereof and/or;
(10) the physiological condition comprises a cardio-respiratory condition and/or;
(11) the cardio-respiratory condition is selected from the group consisting of heart attack, stroke, heart failure, arrhythmia, coronary artery disease, peripheral artery disease, aortic disease, congenital heart disease, chronic bronchitis, chronic obstructive pulmonary disease, and congestive heart failure and/or;
(12) the physiological condition comprises a sleep disorder and/or;
(13) the predicting is based on a moving-average model and/or;
(14) the moving-average model is trained to assign a greater weight or influence to one or more recent data in the historical sensor data set as compared to one or more older data in the historical sensor data set and/or;
(15) the one or more recent data comprises at most about 1, 2, 3, 4, 5, 6, 7, 8, 9, 10, 15, or 20 latest uses of the article of furniture by the user and/or;
(16) the moving-average model comprises one or more members selected from the group consisting of Exponential Moving Average (EMA), Simple Moving Average (SMA), Weighted Moving Average (WMA), Double Exponential Moving Average (DEMA), Triple Exponential Moving Average (TEMA), Smoothed Moving Average (SMMA), Adaptive Moving Average (AMA), Linear Weighted Moving Average (LWMA), Hull Moving Average (HMA), or a modification thereof and/or;
(17) the physiological condition comprises a plurality of physiological conditions, and wherein the moving-average model comprises a different moving-average model for each of the plurality of physiological conditions and/or;
(18) in the anomaly, the new data and the future data are different by a predetermined threshold metric and/or;
(19) the determined difference is characterized by deviation of the new data by at least about 1 standard deviation from the future data and/or;
(20) the determined difference is characterized by deviation of the new data by at least about 2 standard deviations from the future data and/or;
(21) the predicting is performed during the user's current use of the article of furniture, wherein the new data is collected during the current use and/or;
(22) the predicting is performed at least about 1, 2, 3, 4, 5, 10, 20, or 30 time(s) per minute and/or;
(23) the predicting is performed at most about 30, 20, 10, 5, 4, 3, 2, or 1 time(s) per minute and/or;
(24) the predicting is performed substantially in real-time relative to collection of the new data and/or;
(25) the historical sensor data set and the new data are measured by the same sensor and/or;
(26) the historical sensor data set and/or the new data is measured by a biological sensor and/or;
(27) the biological sensor comprises a pressure sensor, optionally wherein the pressure sensor comprises a piezoelectric sensor and/or;
(28) the biological sensor comprises a temperature sensor and/or;
(29) the determining the difference is performed subsequent to determining that the user is sleeping on the article of furniture and/or;
(30) the method further comprises generating a digital report based on the anomaly in the physiological condition of the user and/or;
(31) the method further comprises displaying the digital report on a graphical user interface (GUI) of a user device.
Embodiment 14. A system comprising: a computer processor and a computer memory coupled thereto, wherein the computer memory comprises a machine executable code that, upon execution by the one or more computer processors, implements the method of embodiment 13.
Embodiment 15. A computer-implemented method for detecting a condition of a user of an article of furniture, the method comprising: (a) detecting a plurality of sensor data signatures from a sensor data set associated with a user of an article of furniture; (b) identifying one or more acceptable quality sensor data signatures from the plurality of sensor data signatures via a signal quality filtering algorithm; and (c) determining presence of the physiological condition of the user based on the one or more acceptable quality sensor data signatures identified,
optionally wherein:
(1) the identifying comprises (i) keeping high quality sensor data signatures from the plurality of sensor data signatures and (ii) filtering out low quality sensor data signatures from the plurality of sensor data signatures and/or;
(2) the identifying is performed via a signal quality processing model and/or;
(3) the signal quality processing model is based on Signal-to-Noise Ratio detection, Peak detection, Spectral analysis-based detection, Correlation-based detection, Adaptive thresholding detection, or any combination thereof and/or;
(4) the signal quality processing model is based on low-pass filter, high-pass filter, band-pass filter, band-reject filter, or any combination thereof and/or;
(5) the detecting is performed via a machine learning model and/or;
(6) the machine learning model is trained on clinical data, optionally wherein the clinical data comprises electrocardiogram data and/or;
(7) the machine learning model comprises a deep learning model, optionally wherein the deep learning model comprises a convolutional neural network (CNN) and/or;
(8) the detecting and the identifying are performed by different computer algorithms and/or;
(9) the method further comprises identifying, from the plurality of sensor data signatures, a plurality of patterns associated with the physiological condition, wherein the determining is based on the plurality of patterns and/or;
(10) a pattern of the plurality of patterns comprises a fluctuation between two or more consecutive sensor data signatures and/or;
(11) the method further comprises classifying each pattern of the plurality of patterns to one of (i) a normal pattern that is not associated with the physiological condition and (ii) an abnormal pattern that is associated with the physiological condition, wherein the determining is based on the abnormal pattern in absence of the normal pattern and/or;
(12) the classifying is based in part on a time-series filter and/or;
(13) the presence of the physiological condition is determined when a frequency of the abnormal pattern (loading rate) meets a predetermined threshold frequency value, optionally wherein the predetermined threshold frequency value is at least about 1%, 2%, 3%, 4%, 5%, 6%, 7%, 8%, 9%, or 10% and/or;
(14) the article of furniture is a mattress, a blanket, a pillow, or a cover thereof and/or;
(15) the sensor data set is collected by a biological sensor while the user is sleeping on the article of furniture and/or;
(16) the biological sensor is a part of the article of furniture and/or;
(17) the biological sensor comprises a pressure sensor, optionally wherein the pressure sensor comprises a piezoelectric sensor and/or;
(18) the biological sensor comprises a temperature sensor and/or;
(19) a data signature of the plurality of sensor data signatures comprises respiratory rate, breath cycle, heart rate, heart rate variability, vibration, or any combination thereof and/or;
(20) the data signature comprises a heartbeat and/or;
(21) the physiological condition comprises a cardio-respiratory condition and/or;
(22) the cardio-respiratory condition is selected from the group consisting of heart attack, stroke, heart failure, arrhythmia, coronary artery disease, peripheral artery disease, aortic disease, congenital heart disease, chronic bronchitis, chronic obstructive pulmonary disease, and congestive heart failure and/or;
(23) the physiological condition comprises a sleep disorder and/or;
(24) the method further comprises generating a digital report based on the presence of the physiological condition of the user and/or;
(25) The method further comprises displaying the digital report on a graphical user interface (GUI) of a user device.
Embodiment 16. A system comprising: a computer processor and a computer memory coupled thereto, wherein the computer memory comprises a machine executable code that, upon execution by the one or more computer processors, implements the method of embodiment 15.

Systems and methods of the present disclosure may be combined with or modified by additional systems comprising an article of furniture (e.g., a bed device) and methods of use thereof. For example, systems and methods of detecting a biological signal or a condition (e.g., a sleep disorder) of a user of an article of furniture, regulating a temperature or configuration of an article of furniture, regulating a biological signal or condition (e.g., a sleep disorder) of the user on an article of furniture, regulating operation of other devices operatively coupled to the article of furniture are described in U.S. Patent Publication No. 2015/0351556 (“BED DEVICE SYSTEM AND METHODS”), U.S. Patent Publication No. 2016/0128488 (“APPARATUS AND METHODS FOR HEATING OR COOLING A BED BASED ON HUMAN BIOLOGICAL SIGNALS”), U.S. Patent Publication No. 2017/0135882 (“ADJUSTABLE BEDFRAME AND OPERATING METHODS FOR HEALTH MONITORING”), U.S. Patent Publication No. 2017/0135632 (“DETECTING SLEEPING DISORDERS”), U.S. Patent Publication No. 2020/0405998 (“SLEEP POD”), and U.S. Patent Publication No. 2021/0315389 (“SYSTEMS AND METHODS FOR REGULATING A TEMPERATURE OF AN ARTICLE OF FURNITURE”), each of which is incorporated herein by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for detecting an anomaly in a physiological condition of a user of an article of furniture, the method comprising:
(a) generating future sensor data or predictive analysis thereof based on a historical sensor dataset obtained from a plurality of sleep sessions of at least one user resting or sleeping on an article of furniture, the article of furniture comprising one or more sensors to measure biological signals of the at least one user to generate the historical sensor dataset, and wherein the future sensor data or predictive analysis thereof is indicative of at least one physiological condition of an individual user;
(b) obtaining current sensor data for the individual user resting or sleeping on the article of furniture;
(c) detecting a difference between (i) the generated future sensor data or predictive analysis thereof and (ii) the current sensor data, wherein the difference is indicative of an anomaly in the at least one physiological condition of the individual user; and
(d) generating a health risk alert in response to the detected difference indicative of the anomaly in the at least one physiological condition of the individual user.

2. The method of claim 1, wherein the historical sensor dataset is obtained from a plurality of sleep sessions.

3. The method of claim 1, wherein the at least one user is the individual user.

4. The method of claim 1, wherein the article of furniture is a mattress, a blanket, a pillow, or a cover thereof.

5. The method of claim 1, wherein the plurality of sleep sessions of the at least one user comprises a plurality of consecutive sleep sessions.

6. The method of claim 1, further comprising identifying from the historical sensor dataset one or more sensor data signatures associated with the at least one physiological condition of the individual user, wherein the generating the future sensor data or predictive analysis thereof is based on the one or more sensor data signatures.

7. The method of claim 6, wherein the one or more sensor data signatures comprise a pattern of sensor data or a range of sensor data values.

8. The method of claim 6, wherein the one or more sensor data signatures comprise respiratory rate, breath cycle, heart rate, heart rate variability (HRV), vibration, perspiration, blood pressure, body temperature, or a combination thereof.

9. The method of claim 6, wherein the one or more sensor data signatures comprise the respiratory rate, the heart rate, or the HRV.

10. The method of claim 9, wherein the one or more sensor data signatures comprise at least two members selected from the group consisting of the respiratory rate, the heart rate, and the HRV.

11. The method of claim 1, wherein the at least one physiological condition comprises a cardio-respiratory condition.

12. The method of claim 11, wherein the cardio-respiratory condition is selected from the group consisting of heart attack, stroke, heart failure, arrhythmia, coronary artery disease, peripheral artery disease, aortic disease, congenital heart disease, chronic bronchitis, chronic obstructive pulmonary disease, and congestive heart failure.

13. The method of claim 1, wherein the at least one physiological condition comprises a sleep disorder.

14. The method of claim 13, wherein the sleep disorder is selected from the group consisting of insomnia, sleep apnea, snoring, circadian rhythm sleep disorder, and restless legs syndrome.

15. The method of claim 1, wherein the predicting is based on a moving-average model.

16. The method of claim 15, wherein the moving-average model assigns a greater weight or influence to one or more recent data in the historical sensor data set as compared to one or more older data in the historical sensor data set.

17. The method of claim 15, wherein the moving-average model comprises one or more members selected from the group consisting of Exponential Moving Average (EMA), Simple Moving Average (SMA), Weighted Moving Average (WMA), Double Exponential Moving Average (DEMA), Triple Exponential Moving Average (TEMA), Smoothed Moving Average (SMMA), Adaptive Moving Average (AMA), Linear Weighted Moving Average (LWMA), and Hull Moving Average (HMA).

18. The method of claim 15, wherein the at least one physiological condition comprises a plurality of physiological conditions, and wherein the moving-average model comprises a different moving-average model for each of the plurality of physiological conditions.

19. The method of claim 15, wherein the moving-average model for the at least one physiological condition comprises different moving-average models for different days of the week.

20. The method of claim 19, wherein the moving-average model for the at least one physiological condition comprises different moving-average models for a weekday and a weekend.

21. The method of claim 15, further comprising generating a personalized moving-average model for the individual user.

22. The method of claim 1, wherein one or more data points in the historical sensor dataset is labeled with at least one event associated with a time when the one or more data was generated.

23. The method of claim 22, wherein the one or more data points is manually labeled by the at least one user via a graphical user interface (GUI) of a user device associated with the at least one user.

24. The method of claim 1, wherein the detected difference between (i) the generated future sensor data or predictive analysis thereof and (ii) the current sensor data indicates being different by a predetermined threshold metric indicates the anomaly.

25. The method of claim 24, wherein the predetermined threshold metric is characterized by a deviation of the current sensor data by one or more standard deviations from the generated future data or predictive analysis thereof.

26. The method of claim 1, wherein the generating the future data or predictive analysis thereof is performed during the individual user's concurrent use of the article of furniture.

27. The method of claim 1, wherein the generating the future data or predictive analysis thereof is performed substantially in real-time relative to the obtaining of the current sensor data.

28. The method of claim 1, wherein the detecting the difference is performed subsequent to determining that the individual user is sleeping on the article of furniture.

29. The method of claim 1, further comprising displaying the health risk alert on a graphical user interface (GUI) of a user device associated with the individual user.

30. A system comprising: a computer processor and a computer memory coupled thereto, wherein the computer memory comprises a machine executable code that, upon execution by the one or more computer processors, implements the method of claim 1.
